SEQUENCE LISTING (2 sequences)

SEQ ID NO: 1 - DNA, 170 bp, synthetic construct
  misc_feature 1..170: note = Synthetic
  source 1..170: mol_type = other DNA
aaaaaaaaaaaaaaaaaaaaaaaaaaaaaaaaaaacccccaaaaacccccaaaaacccccaaaaacccccaaaaacccccaaaaaaaaaaaaaaaaaaaaaaaaaaaaaaaaaaaaaaaatccatggtcttgcctacgcacgcagctcacaaaaaaaaaaaaaaaaaaaa

SEQ ID NO: 2 - DNA, 30 bp, synthetic construct
  misc_feature 1..30: note = Synthetic
  source 1..30: mol_type = other DNA
gtgagctgcgtgcgtaggcaagaccatgga

(19) United States
(12) Patent Application Publication
(10) Pub. No.: US 2024/0060933 A1
(43) Pub. Date: Feb. 22, 2024

(54) MAGNETIC FORCE CONTROL OF POLYMER TRANSLOCATION THROUGH NANOPORES

(71) Applicant: ELECTRONIC BIOSCIENCES, INC., San Diego, CA (US)

(72) Inventors: Eric PETERSON, Salt Lake City, UT (US); John WISNIEWSKI, South Salt Lake, UT (US); Aaron FLEMING, Midvale (CA); Sean GERMAN, Salt Lake City, UT (US); Michael KRUPKA, San Diego, CA (US); Eric N. ERVIN, Salt Lake City, UT (US)

(21) Appl. No.: 18/133,258

(22) Filed: Apr. 11, 2023

Related U.S. Application Data
(60) Provisional application No. 63/329,651, filed on Apr. 11, 2022.

(51) Int. Cl. G01N 27/447; C12Q 1/6869

(52) U.S. Cl. CPC G01N 27/44786; C12Q 1/6869; G01N 27/44791; C12Q 1/6806

(57) ABSTRACT

In alternative embodiments, the technology described herein is directed in part to combined magnetic tweezer-nanopore devices, in part to combined magnetic tweezer-nanopore sequencing of polymers, and in part to preparation of polymers for combined magnetic tweezer-nanopore sequencing.

### RELATED APPLICATIONS

This U.S. Utility patent application claims the benefit of priority under 35 U.S.C. § 119(e) of 35 U.S.C. § 119(e) of U.S. Provisional Patent Application Ser. No. (USSN) 63/329,651 filed on Apr. 11, 2022. The aforementioned application is expressly incorporated herein by reference in its entirety and for all purposes. All publications, patents, patent applications cited herein are hereby expressly incorporated by reference for all purposes.

### REFERENCE TO ELECTRONIC SEQUENCE LISTING

The application contains a Sequence Listing which has been submitted electronically in .XML format and is hereby incorporated by reference in its entirety. Said .XML copy, created on Jun. 9, 2023, is named “7130.150349US.xml” and is 4,245 bytes in size. The sequence listing contained in this .XML file is part of the specification and is hereby incorporated by reference herein in its entirety.

### TECHNICAL FIELD

In alternative embodiments, the technology described herein is directed in part to combined magnetic tweezer-nanopore devices, in part to combined magnetic tweezer-nanopore sequencing of polymers, and in part to preparation of polymers for combined magnetic tweezer-nanopore sequencing.

### BACKGROUND

Nanopore-based strand sequencing of DNA, RNA, proteins, or other polymers requires methods which both control the translocation velocity of the polymer through the nanopore and apply tension to the polymer to maintain it in a stretched, extended conformation as it translocates through the sensing zone of the nanopore. Applying tension reduces diffusion and/or the formation of folded, coiled, twisted, and/or compressed polymer configurations, which interfere with the sequence-dependent current response associated with the polymer translocating through the nanopore. Additionally, sequencing accuracy of a polymer can be significantly improved by repeatedly re-reading the same polymer sequence multiple times, iteratively, via the same nanopore reader, by using the individual reads to statistically determine the a high-accuracy consensus sequence determination. Currently, nanopore based strand-sequencing often is carried out using a motor enzyme to hold onto and control the motion of a polymer through a nanopore reader. The polymer held by the motor enzyme is stretched tight by the significant electrophoretic force (8 to 60 pN) pulling it through the nanopore reader, which prevents diffusion and coiling within the nanopore. By minimizing this diffusive motion, the applied tension is able to reduce measurement noise to enable more accurate sequencing of the polymer. However, the use of the motor enzyme limits the number of times the polymer can be read, typically to just one time.

### SUMMARY

In alternative embodiments, provided are nanopore polymer sequencing systems, comprising: a chip comprising a substrate, a layer disposed on the substrate, a sensor site disposed in the layer, an electrode disposed at the sensor site, an amplifier in connection with the electrode; and a nanopore disposed in the sensor site; a magnet disposed in a cis position relative to the nanopore; and, a circuit comprising the electrode, an amplifier in connection with the electrode, a data acquisition (DAQ) system in connection with the amplifier, a bias module in connection with the amplifier, and a control module in connection with the bias module and the DAQ system; wherein the circuit is configured to maintain a voltage bias level at the sensor site based on a state of the nanopore. In alternative embodiments, the circuit includes a reference electrode, and the amplifier often is in connection with the reference electrode; and in alternative embodiments, a reference electrode is an external electrode, optionally disposed outside of a well or chamber, optionally disposed on or in the chip, and optionally disposed in a cis position relative to the nanopore.

Motor-enzyme based sequencing schemes have limitations, including the inability to sequence a single polymer more than one or two times, errors induced by polymer base skipping or backsliding in the motor enzyme, stochastic motion of the motor enzyme causing variable accuracy of the associated base calling, and poor sequencing accuracy of polymers containing repeat sequences, and degradation of the motor enzyme over time.

In alternative embodiments, systems and mechanisms (summarized in FIG. 1) are provided for using an external magnetic field to control the motion of polymers (including DNA, RNA, and proteins) that are tethered to paramagnetic nano- or microparticles, within nanopore readers, in combination with high speed voltage biasing (for example, field-programmable gate array controlled voltage biasing), for the purpose of (1) ensuring that the electrophoretic and/or the electroosmotic force pulling the polymer into and through the nanopore and the opposing magnetic force elongates/stretches the polymer within the nanopore reader, a requirement for nanopore sequencing; (2) controlling/slowing the rate of translocation through the nanopore without the use of an enzyme/motor, using voltage bias induced electrophoresis and/or electroosmosis opposed by the magnetophoretic force; (3) iteratively flossing/multipassing the strand back-and-forth (i.e. re-reading) through the nanopore reader, sequencing the polymer in either translocating direction (i.e. into the nanopore or out of the nanopore), or both (FIG. 2).

In each iteration (for example, iteration (1) and iteration (2) in specific implementations described herein) the polymer generally translocates through the nanopore reader in a direction opposite the direction in the preceding iteration or following iteration. For example, a polymer can translocate through a nanopore reader in a first iteration (iteration (1)) in one direction (for example, in a trans-to-cis direction, which also is referred to herein as a cis direction) and then subsequently the polymer can translocate through the nanopore reader in a second iteration (iteration (2)) in an opposite direction (for example, in a cis-to-trans direction, which also is referred to herein as a trans direction). Often two or more iterations are implemented. Two iterations can be considered a cycle and multiple cycles can be implemented.

Also provided are methods for attaching target polymers to individual magnetic particles, such that they can be used with and sequenced by the system. In addition, methods are also included to ligate an adapter molecule onto the end of the target polymer, opposite of the end attached to the magnetic particle, to help facilitate its capture by the nanopore, and/or its associated flossing/multipassing. An adapter molecule sometimes is referred to as an “adaptor” or “tag” molecule herein. A target polymer for sequencing also is referred to as an input polymer (for example, input nucleic acid, input polypeptide).

In certain aspects, provided is a method that includes (i) conjugating a magnetic particle to the end of a polymer, thereby generating a modified polymer; (ii) electrophoretically and/or electroosmotically capturing and retaining the modified polymer by a nanopore reader (referred to as a nanopore hereafter), through which the entirety of the modified polymer cannot translocate because the magnetic particle is too large to fit through the nanopore; and (iii) applying a magnetic field that translocates the modified polymer, which is under tension, through the nanopore in a direction opposite of an imparted electrophoretic/electroosmotic force. The modified polymer translocates through the nanopore in (iii) by the magnetic force imparted by the magnetic field, which pulls the magnetic particle in the direction opposite of the imparted electrophoretic/electroosmotic force. Also, tension is exerted on the polymer by opposing forces on the polymer, such as a magnetic force in one direction (for example, trans-to-cis direction) and an electrophoretic/electroosmotic force in an opposing direction (for example, cis-to-trans direction).

In certain implementations, a method includes (iv) adjusting the electrophoretic/electroosmotic force on the modified polymer whereby the polymer translocates through the nanopore in a direction opposite the direction in (iii), while under tension. In certain instances, a method includes, after the modified polymer exits the nanopore or sensing zone of the nanopore, or almost exits the nanopore, adjusting the electrophoretic/electroosmotic force on the modified polymer whereby the modified polymer is controllably translocated back through the nanopore in a direction opposite the direction in (iii), while under tension.

In certain implementations, (iii) and (iv) optionally are repeated one or more times. In certain instances, a method includes (v) measuring current as a function of time through the nanopore (while the polymer is translocating in the direction of (iii) and/or (iv)), optionally measuring other data associated with the modified polymer translocating through the nanopore, and determining the sequence of the polymer based on the data. In certain instances the polymer is DNA, RNA, proteins, peptides, analogs, and/or combinations thereof.

Certain implementations are described further in the following description, examples and claims, and in the drawings.

The details of one or more exemplary embodiments of the invention are set forth in the accompanying drawings and the description below. Other features, objects, and advantages of the invention will be apparent from the description and drawings, and from the claims.

All publications, patents, patent applications cited herein are hereby expressly incorporated by reference in their entireties for all purposes.

### DESCRIPTION OF DRAWINGS

The drawings illustrate certain implementations of the technology and are not limiting. For clarity and ease of illustration, the drawings are not made to scale and, in some instances, various aspects may be shown exaggerated or enlarged to facilitate an understanding of particular implementations.

FIG. 1 shows an overview of an exemplary magnetic multipass sequencing device implementation; the exemplary sequencing device includes a chip-base microwell platform with an electrode at the bottom of the microwell. A membrane is placed over the microwell, which holds and contains a nanopore reader (a solid-state nanopore within a given substrate also can be utilized). A mechanism and apparatus for applying an AC or DC bias across the nanopore and for monitoring the conductance or electrical properties of the nanopore reader is included. A polymer tagged with a magnetic nanoparticle is driven into and trapped within the nanopore reader. A mechanism and apparatus for applying a magnetic field to the sequencing device is included. The applied magnetic field pulls the tagged polymer out of the nanopore while an electrophoretic and/or electroosmotic force pulls the tagged polymer into the pore. The tagged polymer is sequenced; as the polymer is controllably translocated into the nanopore, out of the nanopore, or both, the obtained translocation signature of the polymer is used to determine the sequence of the polymer.

FIG. 2A-2D. show an overview of an exemplary protocol implementation for capturing and repeatedly sequencing as magnetically tagged polymer via a nanopore reader with the magnetic multipass sequencing device/system. As illustrated in FIG. 2A, polymers are first captured within the nanopore reader by the electrophoretic force produced by the applied voltage bias. As illustrated in FIG. 2B, a magnetic force is applied to the polymer through the magnetic particle tag by activating a magnetic field gradient using the magnetic tweezer device. As illustrated in FIG. 2C, the voltage bias is reduced so that the electrophoretic force pulling the polymer into the nanopore reader is exceeded by the magnitude of the magnetic force pulling it out the top of the reader, causing the polymer to migrate out of the reader. As illustrated in FIG. 2D, by increasing the voltage bias so that the electrophoretic force exceeds the magnetic force, the polymer translocates back into the reader at a controllable velocity allowing it to be sequenced. During this process, polymer is always held taught and fully extended by the opposing forces between the magnetic particle and the nanopore reader.

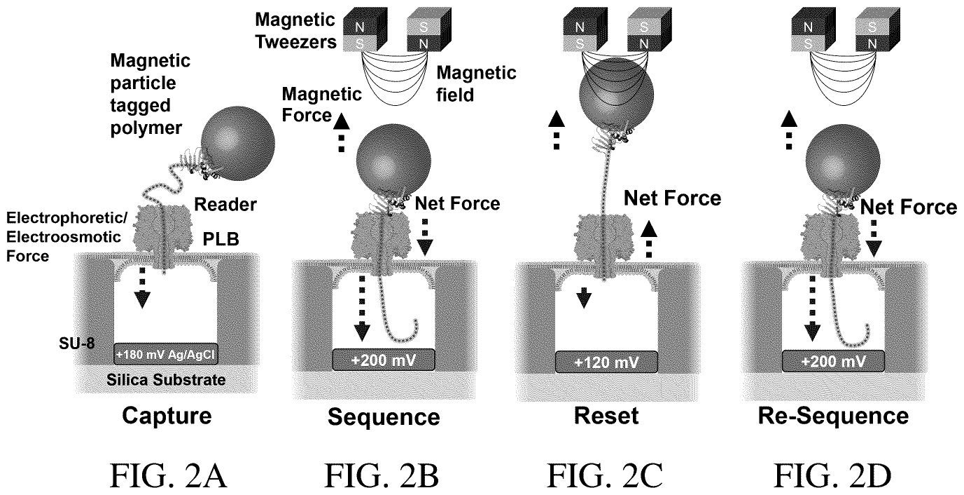

FIG. 3A-3E illustrate arrangements of representative fixed magnet implementations in a magnetic tweezer apparatus implementation. FIG. 3A provides definitions of magnet dimensions for rectangular (above) and cylindrical magnets (below). FIGS. 3B-3E illustrate representative arrangements of fixed magnets, including a Single magnet, a pair of vertically aligned magnets, a pair of horizontally aligned magnets, and an electromagnet, respectively.

FIG. 4A-4C illustrate exemplary arrangements of fixed magnets in magnetic tweezer device implementations with microstructures designed to increase the magnetic field gradient. FIG. 4A illustrates a Mu-metal strip near the magnetic particle sample, FIG. 4B illustrates a magnetic tip, and FIG. 4C illustrates a focusing yoke.

FIG. 5 illustrates the theoretical magnetic force distribution versus distance, z, on 2.8 micron diameter M270 magnetic particles (Thermo Fisher), from a pair of Neodymium cube magnets of 1 mm in length with a 0.25 mm gap between them (blue), and a pair of 2 mm Neodymium magnets with a 0.5 mm gap between them. The force is calculated based on methods described by Lipfert (2009).

FIG. 6 illustrates an exemplary magnetic force distribution versus distance z from a 1 mm diameter, 5 mm long cylindrical Neodymium magnet on a 2.8 micron diameter M270 magnetic particle (Thermo Fisher). The force is calculated using the magnetic field distribution around a cylindrical magnet.

FIG. 7 illustrates an exemplary implementation for anchoring polymers onto magnetic particles. Magnetic particles coated with a capture functional group (for example, streptavidin, antibodies, reactive chemical group, and the like), are coated with polymer molecules conjugated to a complementary reactive group (for example, biotin, antigen, complementary reactive group, respectively).

FIG. 8 illustrates an exemplary generalized implementation for library preparation in which a poly-A tail and magnetic particle are installed on dsDNA for sequencing. Two nonidentical adapters are ligated onto opposite ends of the double stranded genomic DNA. The “capture” adapter contains a polyelectrolyte capture tail (for example, poly-A₅₀ homopolymer ssDNA), and the “anchoring” adapter contains a functional group for attachment to the magnetic particle (reactive group for chemical ligation or an affinity tag). These adapters are installed such that the capture tail and the magnetic particle attachment group are both ligated onto the same ssDNA strand in the duplex which serves as the sequencing target. This way, the opposing magnetic force and electrophoretic/electroosmotic force both pull on and stretch target sequence. Adapters are composed of a short 18-30 bp duplex DNA or RNA sequence, each of which can contain a complementary sequence followed by a single-stranded tail which serve purposes described in Figure Y.

FIG. 9A-9B illustrate exemplary implementations for adaptor ligation to a target dsDNA digested with a fragmentase enzyme to yield sequence specific ends for the adaptors. A fragmentase enzyme is used to cleave genomic DNA and produce fragments single stranded sticky ends. Fragments from the interior of the DNA contain distinct sticky ends on the 5′ end which is target for ligation with adapters. There are two distinct adapter designs. FIG. 9A illustrates a method for targeting a single strand for sequencing. In this design, one strand in the capture adapter duplex contains a capture tail for entry into the nanopore reader, and the other strand contains a bulky blocking group (for example, biotin/streptavidin complex or a G-quadruplex) that prevents entry of that strand into the reader. In the anchoring adapter duplex, one strand contains a functional group for attachment to the particle, while the other contains a blocking group to prevent entry into the reader. The capture tail and the particle attachment group are attached to the same strand in the duplex, while the blocking groups are both attached the complementary strand. FIG. 9B illustrates a method for both strands to be sequenced. In this design, both strands in the capture adapter duplex contain capture tails (for example, 30-50 nt poly-A ssDNA), and both strands of the anchoring adapter duplex contain functional groups for ligation to magnetic particles. This way, both strands are anchored to the magnetic particle and contain capture tails for capture and entry into nanopore readers.

FIG. 10 illustrates an exemplary chemoenzymatic implementation for adaptor ligation to a target dsDNA sample. In part1, the 5′ ends of the target dsDNA are modified using T4 RNA Ligase, Mth Ligase, T4 polynucleotide Kinase, or other enzymatic methods to install a reactive R group on the 5′ ends of the target dsDNA using an R-modified substrate. In part 2, the reactive R group on the DNA is ligated to magnetic particles and blocking groups which have been modified with the reaction partner group, R′. This reaction can proceed with an excess of blocking group relative to magnetic particles to prevent ligation of magnetic particles to each end of the target dsDNA, which is undesirable. In part 3, a poly-A ssDNA tail is synthesized on the remaining 3′ ends of the target dsDNA using TdT addition to produce a capture tail for reader entry. The poly-A capture tail distal to the magnetic particle is significantly more likely to be captured than the poly-A capture tail proximal to the magnetic particle, as it is more accessible with less steric hindrance from the magnetic particle.

FIG. 11A-11B illustrate exemplary implementations for rigidifying ssRNA to a longer persistence length. FIG. 11A illustrates rigidification by annealing with short random complementary sequences. FIG. 11B illustrates rigidification via synthesis of a complementary cDNA strand using a reverse transcriptase.

FIG. 12A-12B illustrate exemplary implementations for installing poly-A tails on RNA to be sequenced. FIG. 12A shows an exemplary process employing enzymatic attachment of polymer tails. This process includes enzymatic addition of a poly-A tail to the 3′ end with an RNA polymerase, which can be used as a capture adapter directly (as shown), or further modified for ligation to magnetic particles. The 5′ end can be modified using a capping enzyme complex to install a reactive group for attachment to magnetic particles (as shown) or polymer capture adapters. FIG. 12B illustrates a process in which adapters can be installed using enzymatic attachment of orthogonal reactive groups on ends of RNA followed by chemical ligation of capture or anchoring adapters containing the respective reactive groups.

FIG. 13A-13B illustrate exemplary implementations for targeting specific regions of sheared genomic DNA for installation of capture and anchoring adapters. FIG. 13A illustrates an exemplary approach using Cas9 nuclease and a gRNA to target specific regions, where in part 1 the 3′ ends of the DNA are capped with ddNTPs to prevent ligation; in part 2 the targeted region is cut out using Cas9 gRNA targeted cleavage; in part 3, a poly-A tail is installed on the 3′ end with an optional terminal biotin for ligation to magnetic particles; and in part 4, a reactive group is installed on the 5′ end for ligation to a polymer capture tail or magnetic particle. FIG. 13B illustrates an exemplary implementation for using designer ssDNA probes and circLigase to target a specific strand. In part 1, genomic DNA fragments are head denatured; in part 2, ssDNA probes are annealed onto positions flaking the region of interest; in part 3; ssDNA tail is ligated to target strand using CircLigation; in part 4, melted genomic complementary strand is digested and removed using Exol; in part 5, a new strand complementary to the genomic region of interest is synthesized in the gap between the probes; in part 6, single stranded loops in probes are cleaved open at dU sites; and in part 7, the final capture adapter and anchoring adapter structure is produced, which includes a homopolymer tail, a base-modified biotin for magnetic particle attachment, and G-quadruplex blocking groups on the complementary strand.

FIG. 14A-14F illustrate exemplary implementations in which adapters allow a protein to be electrophoretically captured by a nanopore reader and anchored onto a magnetic particle. FIG. 14A (Upper) illustrates a DNA double-stranded elongation capture adapter that can be attached to the ends of a protein, either the N terminus, C terminus, with a single-stranded tail for nanopore entry. FIG. 14A (Lower) illustrates a double-stranded anchoring adapter has a reactive group for attachment to the N- or C-terminus of the protein and a biotin, or other affinity group for attachment to the magnetic particle. The complementary duplex strand is terminated in a blocking group (for example, G-quadruplex) to prevent entry into nanopore. FIG. 14B (Upper) illustrates a single α-helix (SAH) peptide based capture tag with reactive group for attachment to N or C terminus of protein, rigid α-helix and unstructured homopolymer tail for entry into nanopore. FIG. 14B (Lower) illustrates a SAH peptide based anchoring adapter has a reactive group for attachment to C- or N-terminus of the protein and a terminal biotin or other affinity tag for attachment to magnetic the particle. FIG. 14C illustrates an exemplary process in which a protein can be attached to a capture tag on one terminus, and directly bound to a magnetic particle on the other end with no anchoring adapter. FIGS. 14D-14FD illustrate capture tags attached to the ends of the protein can include two DNA-based adapters as shown in FIG. 14D, or two peptide-based adapters as shown in FIG. 14E, or a mix of peptide and DNA based adapters as shown in FIG. 14F.

FIG. 15A-15B illustrate exemplary implementations in which magnetic particles are modified with passivation agents, i.e., PEG, to reduce and/or prevent nonspecific adsorption. FIG. 15A illustrates a particle with an attached target polymer coated with surfactant designed to minimize nonspecific adsorption. FIG. 15B illustrates attachment of amine-PEG-hydroxy to streptavidin coated magnetic particles, followed by attachment of streptavidin-modified DNA to the streptavidin groups.

FIG. 16A-16B illustrate exemplary implementations in which a PLB surface is passivated to reduce and/or prevent nonspecific adsorption. FIG. 16A illustrates introducing PEG-modified phospholipids to the cis chamber aqueous volume and waiting a set time period for the PEG-lipids to partition into the PLB. FIG. 16B illustrates incorporating lipids with reactive groups into the PLB and attaching a PEG polymer modified with a complementary reactive group to the modified lipids. The reactive PEG is introduced to the cis chamber and then reacts with the modified lipids.

FIG. 17A-17C illustrate exemplary implementations in which a hard stop is incorporated on the distal end of the polymer, also known as rotaxane formation. FIG. 17A illustrates hybridization of a hard stop oligonucleotide to a complementary sequence on the polymer that resides in the trans chamber after nanopore capture and threading. FIG. 17B illustrates binding of an affinity tag, streptavidin, to its complementary binding partner, biotin, attached to the distal end of the polymer. FIG. 17C illustrates hybridization of a modified hard stop oligonucleotide to a modified complementary sequence on the target polymer in the trans chamber after nanopore capture and threading, where the modified oligonucleotide includes a chemical group (for example, alkyne) capable of reacting with a counterpart chemical group (for example, azide) to form a covalent bond between the target polymer and hybridized hard stop oligonucleotide.

FIG. 18 illustrates an exemplary FPGA controlled magnetic multipassing sequencing implementation. Part 1 includes introducing a polymer sample to a device and applying a capture voltage bias. Part; 2 includes capturing tagged polymer, triggering a switch to a holding voltage bias. Part 3 includes moving magnetic tweezers into position to apply a magnetic force and apply exit voltage bias. Part 4 shows that when the magnetic force is higher than electrophoretic force, polymer translocates trans to cis. In part 5, the distal end of the polymer reaches the reader and triggers a “sequencing” voltage bias. In part 6, the polymer is sequenced as it translocates into the reader. In part 7, the proximal end of polymer enters reader, triggering a switch to an exit voltage. In part 8, the polymer translocates in the trans-to-cis direction again, and the distal end of polymer in the reader triggers a sequencing voltage bias again. In part 9 the polymer is passed through the reader multiple times for repeat sequencing.

FIG. 19 illustrates an exemplary FPGA controlled magnetic multipassing sequencing implementation for duplex DNA samples tagged with magnetic particles and capture adapters. Part 1 includes introducing a dsDNA sample to a device and applying a capture voltage bias. Part 2 includes capturing the dsDNA, and maintaining a capture voltage bias. Part 3 illustrates that the electrophoretic force drawing the DNA into the reader induces dissociation of the complementary strand. Part 4 shows target DNA is fully dissociated and drawn into the cis portion, but held in the nanopore by the bulky magnetic particle tag. Part 5 illustrates that after dissociating the complementary strand, the voltage is stepped to a holding bias. Part 6 includes applying a magnetic field to apply a magnetic force on the magnetic particle. Part 7 includes applying an “exit” voltage bias, where the magnetic force exceeds the electrophoretic force. Part 8 illustrates that the target DNA translocates in the trans-to-cis direction out of the reader under the magnetic force until it escapes the reader, triggering application of a “sequencing” voltage. Part 9, illustrates that the electrophoretic force under the sequencing voltage induces translocation into the nanopore, in the cis-to-trans direction, enabling sequencing of the DNA. Parts 7-9 can be repeated multiple times for multipass sequencing. FIG. 19 illustrates a FPGA controlled magnetic multipassing sequencing implementation for duplex DNA samples tagged with magnetic particles and capture adapters. Part 1 includes introducing a dsDNA sample to a device and applying a capture voltage bias. Part 2 includes capturing the dsDNA, and maintaining a capture voltage bias. Part 3 illustrates that the electrophoretic force drawing the DNA into the reader induces dissociation of the complementary strand. Part 4 shows target DNA is fully dissociated and drawn into the cis portion, but held in the nanopore by the bulky magnetic particle tag. Part 5 illustrates that after dissociating the complementary strand, the voltage is stepped to a holding bias. Part 6 includes applying a magnetic field to apply a magnetic force on particle. Part 7 includes applying an “exit” voltage bias, where the magnetic force exceeds the electrophoretic force. Part 8 illustrates that target DNA translocates in the trans-to-cis direction out of the reader under the magnetic force until it escapes the reader, triggering application of a “sequencing” voltage. Part 9 illustrates that the electrophoretic force under the sequencing voltage induces translocation into the nanopore, in the cis-to-trans direction, enabling sequencing of the DNA. Parts 7-9 can be repeated multiple times for multipass sequencing.

FIG. 20A-20E illustrate an exemplary DNA sequencing implementation using a magnetic force multipass sequencing device. In FIG. 20A a sample containing a dsDNA attached to magnetic particle and Y-shaped adapter is introduced to the device. FIG. 20B illustrates capturing dsDNA via a capture tail on the Y-shaped adapter in the nanopore reader, and identifying a current signature of the capture tail that triggers a voltage to force dissociation of the complementary DNA strand. FIG. 20C illustrates drawing the DNA through the nanopore reader to dissociate the dsDNA and produce ssDNA for sequencing, identifying a current signature of the magnetic particle reaching the nanopore reader, and triggering the magnetic force and exit voltage. FIG. 20D illustrates applying a force on the magnetic particle with the magnetic tweezers and controlling the magnitude of the electrophoretic force with a FPGA controller to translocate the ssDNA out of nanopore, and simultaneously measuring ionic current with the high-speed FPGA device to sequence the DNA. FIG. 20E illustrates use of an exit signature to trigger a higher sequencing voltage and translocating the ssDNA back into nanopore for repeat sequencing. In FIGS. 20A-20E, the following features are identified by the following callouts (callouts in parentheses): silica substrate (15); polymer substrate (20); PLB (25); well (30); nanopore reader (35); well electrode (40); exterior reference electrode (45); FPGA voltage control and current measurement module (50); power supply (51); computer with control software (52); top plate (70); magnetic tweezer (75); linear moveable mount (76); sample flow in (90); sample flow out (92); trans-to-cis direction (94); cis-to-trans direction (96); Y-shaped DNA capture adapter (200); magnetic particle (201); blocking moiety (202); terminal single stranded polynucleotide, also referred to as “capture tail” (203); Y-shaped DNA anchoring adapter (204); target DNA strand in input nucleic acid (210); DNA strand complementary to target DNA strand (211).

FIG. 21A-21E illustrate an exemplary RNA sequencing implementation using a magnetic force multipass sequencing device. FIG. 20A illustrates introducing a sample containing RNA annealed to cDNA and attached to a magnetic particle and a Y-shaped adapter. FIG. 20B illustrates capturing the RNA in the nanopore reader via a capture tail on Y-shaped adapter, and identifying a current signature of the capture tail which triggers a voltage to force dissociation of cDNA. FIG. 20C illustrates drawing the RNA through the nanopore reader to dissociate the complementary strand and produce the ssRNA for sequencing, and identifying a current signature of the magnetic particle reaching nanopore and triggering a magnetic force and exit voltage. FIG. 20D illustrates applying a force on the magnetic particle with the magnetic tweezers and controlling the magnitude of the electrophoretic force with a FPGA controller to translocate RNA out of nanopore, and simultaneously measuring the ionic current with the high speed FPGA device to sequence the RNA. FIG. 20E illustrates using the exit signature to trigger a higher sequencing voltage to translocate the RNA back into nanopore for repeat sequencing. In FIGS. 21A-21E, the following features are identified by the following callouts (callouts in parentheses): silica substrate (15); polymer substrate (20); PLB (25); well (30); nanopore reader (35); well electrode (40); exterior reference electrode (45); FPGA voltage control and current measurement module (50); power supply (51); computer with control software (52); top plate (70); magnetic tweezer (75); linear moveable mount (76); sample flow in (90); sample flow out (92); trans-to-cis direction (94); cis-to-trans direction (96); magnetic particle (201); blocking moiety (202); terminal single stranded polynucleotide, also referred to as “capture tail” (203); Y-shaped RNA capture adapter (205); Y-shaped RNA anchoring adapter (206); target RNA strand of input nucleic acid (212); complementary DNA strand (213) to target RNA strand.

FIG. 22A-22G illustrate an exemplary protein sequencing implementation using a magnetic force multipass sequencing device. FIG. 22A illustrates introducing a sample containing a protein attached to a magnetic particle and a Y-shaped dsDNA adapter. FIG. 22B illustrates capturing the protein in the nanopore reader via the capture tail on Y-shaped adapter. Identify current signature of capture tail which triggers voltage to force dissociation of cDNA on Y-shaped adapter. FIG. 22C illustrates drawing the adapter into pore to dissociate the adapter cDNA strand, and identifying a current signature of the protein reaching the nanopore reader and triggering a magnetic force and holding voltage. FIG. 22D illustrates applying a force on magnetic particle with the magnetic tweezers to force the protein to denature/linearize. FIG. 22E illustrates applying a voltage to draw protein into the nanopore reader and dissociating the cDNA strand on the anchoring Y-shaped adapter. FIG. 22F illustrates identifying entry of the entire protein into the trans portion of the device and triggering control of the magnitude of electrophoretic force via a voltage bias with a FPGA controller to translocate the protein out of nanopore reader. FIG. 22G illustrates using an exit signature to trigger a higher sequencing voltage to translocate the RNA back into the nanopore reader, simultaneously measuring ionic current with the high speed FPGA device to sequence the protein, and identifying the magnetic particle current signature to trigger an exit voltage for repeat sequencing. In FIGS. 22A-22G, the following features are identified by the following callouts (callouts in parentheses): silica substrate (15); polymer substrate (20); PLB (25); well (30); nanopore reader (35); well electrode (40); exterior reference electrode (45); FPGA voltage control and current measurement module (50); power supply (51); computer with control software (52); top plate (70); magnetic tweezer (75); linear moveable mount (76); sample flow in (90); sample flow out (92); trans-to-cis direction (94); cis-to-trans direction (96); magnetic particle (201); blocking moiety (202); terminal single stranded polynucleotide, also referred to as “capture tail” (203); Y-shaped DNA capture adapter (207); Y-shaped DNA anchoring adapter (208); and target polypeptide also referred to as input polypeptide (214).

FIG. 23A-23F illustrate an exemplary DNA sequencing implementation using a magnetic force multipass sequencing device and a hard stop. FIG. 23A illustrates introducing a sample containing DNA attached to a magnetic particle and a Y-shaped dsDNA adapter and a hard stop capture polynucleotide at or near the distal end. Hard stop oligonucleotides designed to hybridize to the capture polynucleotide of the DNA are present in the trans chamber. FIG. 23B illustrates capturing DNA in the nanopore reader via a capture tail on the Y-shaped adapter, and identifying a current signature of the capture tail, which triggers a waiting period to hybridize the DNA to the hard stop oligonucleotide. FIG. 23C illustrates waiting for a duration of time to allow the hard stop oligonucleotide to hybridize to the DNA. FIG. 23D illustrates drawing the adapter through the nanopore reader to dissociate the cDNA strand, identifying a current signature of DNA reaching nanopore and triggering a magnetic force and holding voltage. FIG. 23E illustrates (i) applying a force on the magnetic particle with the magnetic tweezers and controlling the magnitude of the electrophoretic force with an FPGA controller to translocate DNA out of nanopore, and (ii) simultaneously measuring ionic current with high the speed FPGA device to sequence the DNA, and making use of the hard stop signature to trigger a higher sequencing voltage to translocate the DNA back into nanopore for repeat sequencing. FIG. 23F illustrates applying a higher sequencing voltage to translocate the DNA back into nanopore for repeat sequencing, simultaneously measuring ionic current with the high speed FPGA device to sequence the DNA, and identifying the magnetic particle current signature to trigger an exit voltage for repeat sequencing. In FIGS. 23A-23E, the following features are identified by the following callouts (callouts in parentheses): silica substrate (15); polymer substrate (20); PLB (25); well (30); nanopore reader (35); well electrode (40); exterior reference electrode (45); FPGA voltage control and current measurement module (50); power supply (51); computer with control software (52); top plate (70); magnetic tweezer (75); linear moveable mount (76); sample flow in (90); sample flow out (92); trans-to-cis direction (94); cis-to-trans direction (96); Y-shaped DNA capture adapter (200); magnetic particle (201); blocking moiety (202); terminal single stranded polynucleotide, also referred to as “capture tail” (203); Y-shaped DNA anchoring adapter (204); target DNA strand in input nucleic acid (210); DNA strand complementary to target DNA strand (211); Hard stop capture polynucleotide (230); Hard stop oligonucleotide (231) containing a polynucleotide complementary to the capture polynucleotide (230) linked to the target DNA strand (211); Hard stop structure (232), which is a duplex.

FIG. 24A-24C illustrate an exemplary magnetic tweezer implementations on single and multiplexed microwell sensor chips. FIG. 24A illustrates a single magnetic tweezer with a single microwell sensor, FIG. 24B illustrates a single magnetic tweezer shared with multiple microwell sensors on a multiplexed chip, and FIG. 24C illustrates a multiplexed, individually controlled magnetic tweezers with a multiplexed microwell sensor chip.

FIG. 25 illustrates exemplary components of a magnetic multipassing sequencing system, including the sequencing chip containing a plurality of individually addressable wells, each with an individual working electrode at its bottom and a membrane and nanopore reader at its top. A flow cell or channel which allows a sample or solution to be introduced to the sequencing chip, with and inlet and outlet port. A cover or lid confines the working volume of the sequencing chip. A system or device capable of applying a magnetic field to or across the individual wells of the sequencing chip. A system or device capable of individually applying a bias between the electrodes at the bottom of each well and the reference electrode.

FIG. 26 illustrates an exemplary implementation of magnetic tweezers over a microwell sensor chip with an integrated flow cell, with demonstration of a magnetic particle tagged polymer sample introduction.

FIG. 27 is an exemplary implementation of a magnetic force multipass sequencing device with sensor well, electrodes, flow channel, FPGA controller and support devices, and magnetic tweezers on a moveable mount. In FIG. 27, the following features are identified by the following callouts (callouts in parentheses): multipass sequencing system (10); silica substrate (15); polymer substrate (20); PLB (25); well (30); nanopore reader (35); well electrode (40); exterior reference electrode (45); FPGA voltage control and current measurement module (50); power supply (51); computer with control software (52); top plate (70); magnetic tweezer (75); linear movable mount (76); sample flow in (90); sample flow out (92); trans-to-cis direction (94); and cis-to-trans direction (96).

FIG. 28A-28E illustrate an exemplary implementation in which a hard stop is formed on a target DNA captured in a nanopore reader. FIG. 28A (SEQ ID NO: 1) illustrates the sequence of a target DNA strand. FIG. 28B (SEQ ID NO: 2) illustrates the sequence of complementary DNA strand present in the trans chamber used to form the hard stop. Nucleobases indicated by bold text are incorporated locking nucleotides designed to increase duplex stability when the hard stop oligonucleotide was hybridized to the target DNA. FIG. 28C illustrates a current trace showing entry signature of 3′ end of target DNA (tethered to a magnetic particle on the 5′ end) entering MspA nanopore reader at a −160 mV applied potential. FIG. 28D illustrates a current trace showing voltage step from −120 to +60 mV while target DNA is held in nanopore reader. At +60 mV, current remains in a blocked state, indicating formation of a hard stop that prevents target strand escape. FIG. 28E illustrates a current trace of a voltage switch from −120 mV to +80 mV while a target strand is in the nanopore. The current returned to the open channel current of +80 mV, indicating that the hard stop had been dissociated and the target strand escaped the nanopore. Data was collected with a 20 kHz low pass filter at a data sampling rate of 100 kHz.

FIG. 29 illustrates an exemplary DNA target polymer (sequence shown in FIG. 28A) captured by an MspA nanopore reader, while ligated onto a magnetic particle on the proximal end, with a LNA oligonucleotide duplexed as a hard stop (sequence shown in FIG. 28B) formed on the distal end residing within the trans chamber. The current trace shows voltage steps from −120 mV to +60 mV after a 300 millisecond (ms) delay, and then from +60 mV to −120 mV after a 1 s delay at the indicated arrows. This data set indicated multipassing of the target DNA while using a hard stop to prevent escape into the cis chamber under a positive voltage bias. Data was collected with a 50 kHz low pass filter at a data sampling rate of 250 kHz.

FIG. 30A-30D illustrate exemplary formation and use of a streptavidin hard stop on the distal end of target DNA tethered on the proximal end to a magnetic particle and captured in an MspA nanopore reader. FIG. 30A illustrates the sequence of the single-stranded target DNA. FIG. 30B illustrates the current trace of target DNA in a nanopore reader with a streptavidin hard stop showing a switch from +120 mV to −120 mV applied potential. FIG. 30C and FIG. 30D illustrate additional current trace examples showing a voltage step from +120 to −120 mV while the target DNA is held in nanopore reader. Data was collected with a 20 kHz low pass filter at a data sampling rate of 100 kHz.

Like reference symbols in the various drawings indicate like elements.

### DETAILED DESCRIPTION

### Magnetic Forces on Polymers Tethered to Magnetic Particles

Because polymers targeted for sequencing (for example, DNA, RNA, proteins, and the like) typically do not have strong magnetic properties themselves, the polymers are attached to a magnetically active tag in order to apply force to the polymer with a magnetic field. In order to exert a force on a polymer using an external magnetic field, the polymer can be tagged with a single magnetic particle on one of its ends. If one end of the polymer is captured within a nanopore reader, and the other end of the polymer is tagged with or attached to the magnetic particle, then the external magnetic field can be used to apply a magnetic force pulling the magnetic particle out of the reader, stretching the polymer against an opposing electrophoretic and/or electroosmotic force, into an extended conformation which is needed for nanopore sequencing.

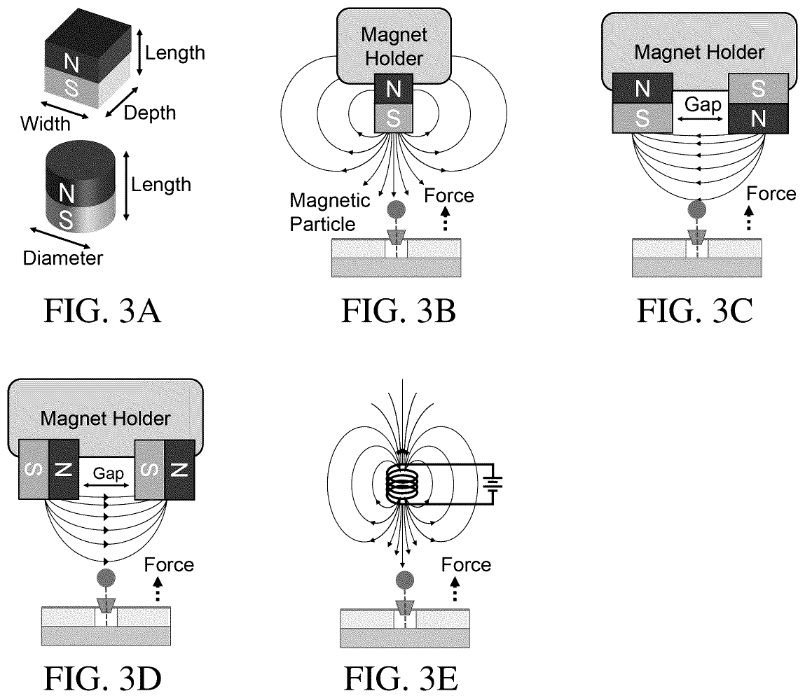

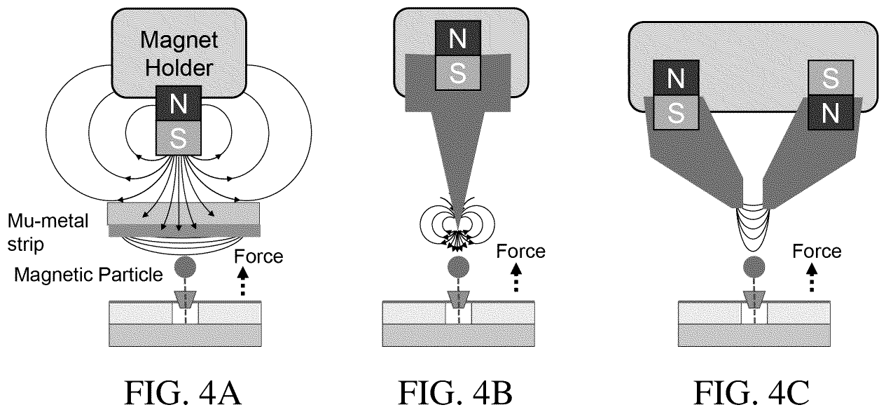

Magnetic particles are widely used for biomolecule separations, where permanent magnets can apply forces in the femptoNewton (fN) to picoNewton (pN) range on the particles, in order to induce rapid migration of magnetic particles over long distances (mm-cm). These magnetic forces can be and are also used to exert forces on polymers tethered to magnetic nano- and microparticles using magnetic tweezer instruments.¹⁻² Magnetic tweezers use small, millimeter to centimeter scale rare earth permanent magnets, either individually, in pairs, or in arrays (FIG. 3 and FIG. 4) to produce a magnetic field distribution able to apply force or torque to magnetic nano- or microparticles. Magnetic tweezers may also be constructed with electromagnets, in which a magnetic field is produced by electrical current applied to a wire coil in contact with a magnetic “core” constructed out of a soft ferromagnetic material (for example, soft iron) (FIG. 3). In a typical or standard magnetic tweezer apparatus, biomolecules attached to magnetic nanoparticles are immobilized onto a surface. The magnitude of the magnetic field decays with distance from the surface of the magnetic tweezers, allowing the force applied (typically in the 10 fN to >100 pN range) to the biomolecules to be controlled via the spacing between the magnetic tweezers and the particles, or in the case of an electromagnet, by the electrical current in the wire coil. Magnetic tweezers have a number of advantages over other single-molecule force techniques (for example, optical tweezers, AFM) for applying force to polymers in nanopore sequencing, including the ability to apply force to many molecules in parallel or within a multiplexed format, apply forces that do not vary with molecule extension (so that the force does not vary significantly as the polymer is drawn in and out of the nanopore reader), and the ability to apply torque or control molecular orientation. Because magnetic tweezers apply uniform forces over a millimeter-scale area, a single magnetic Tweezer device can be used to control forces to arrays of hundreds to thousands of micrometer-scale sensor elements simultaneously, making it possible to multiplex sequencing measurements across many nanopore reader sensors simultaneously. In addition, the magnetic field gradient decays over distances of hundreds of microns, so that the applied force does not significantly vary over the length of micrometer-long polymers like genomic DNA mRNA being pulled out of or into a nanopore reader. Magnetic tweezers thereby can apply a constant force to DNA or other polymers as they are threaded back-and-force through the nanopore, making their translocation easier to control. Finally, magnetic tweezers constructed with pairs of block magnets produce a directional magnetic field that orients the magnetic particle. This construction is useful both in its ability to reduce the rotational diffusion of the magnetic particle and the polymer tethered to it, in addition to providing control of the orientation and torque applied to the polymer by rotating the magnet assembly.

Magnetic particles are typically composed of individual magnetite (Fe₃O₄) nanoparticles, or larger composites that contain multiple magnetic nanoparticles embedded in an inert polystyrene or silica matrix. These particles are available from multiple commercial vendors with a variety of chemical surface modifications for functionalization and attachment to biomolecules. Although bulk magnetite is ferrimagnetic and can adopt a permanent magnetic moment, the magnetic moment of nanocrystalline magnetite realigns randomly due to random thermal fluctuations. However, magnetite nanoparticles are superparamagnetic, and they adopt strong magnetic moments when placed in an external magnetic field.³ Under magnetic fields greater than 0.5 T magnetite nanoparticles reach a maximum saturation magnetization of 50-90 Am² kg⁻¹.³⁻⁶ This induced magnetic dipole moment is subject to a force in a magnetic field gradient, known as magnetophoresis, proportional to the saturation magnetization of the dipole, Mₛ, the mass of the particle, m, and the magnetic field, B, gradient:

F_{mag}=½(Mₛm∇·)B Equation 1

Since the magnitude of the magnetic dipole moment is proportional to the mass of magnetite in the particle, larger particles with higher magnetite content experience stronger forces. In addition, the magnetophoretic force is higher with larger magnetic fields (up to the saturation magnetization of magnetite at a field strength ˜0.5 T) and higher magnetic field gradients. These parameters provide means to control the magnetic force by varying the magnetic particle magnetite content, particle size, and the magnetic properties and geometry of the external magnet tweezers.

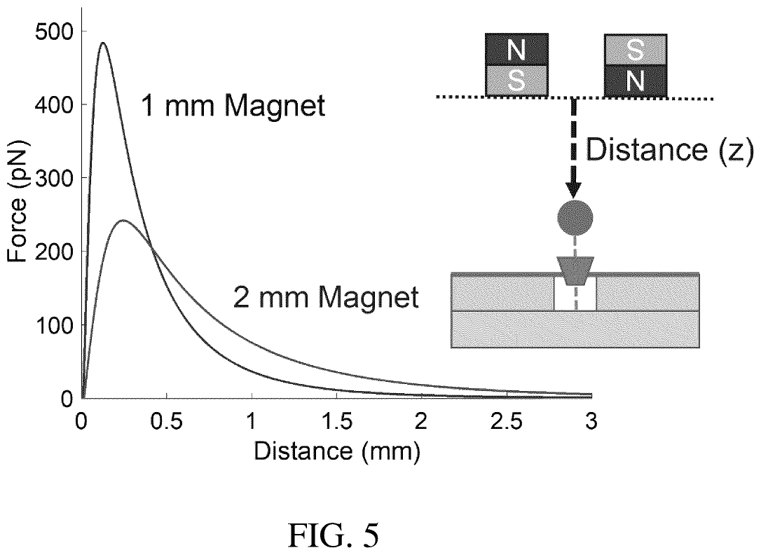

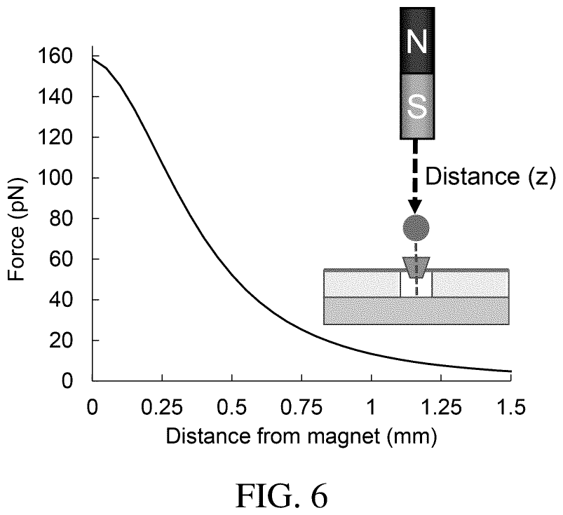

The applied magnetic force can be estimated using models to describe the magnetic field distribution around a pair of block magnets, and the reported magnetization curves of magnetic nanoparticles. Lipfert et al.,² reported a method to calculate the magnetic field distribution along the center of a pair of cubic block magnets. This magnet arrangement produces a uniform, directional magnetic field in the gap between the magnets that allows control of the orientation of the magnetic particle. The magnetic field distribution can be calculated by modeling the magnetic dipoles as current loops and using the Biot-Savart law to integrate their contributions to the magnetic field. Based on these models, the dimensions of the magnet system and the size and magnetic susceptibility of the magnetic particles can be chosen to achieve magnetophoretic forces high enough to oppose the electrophoretic force on the polymer to control its translocation in the nanopore reader and to effectively elongate the polymer for sequencing. FIG. 5 shows the distribution of magnetic field magnitude and magnetophoretic force exerted on 2.8 micrometer Dynabead magnetic particles (Thermo Fisher) for a pair of vertically aligned 1 mm long cube magnets with 250 μm spacing, and 2 mm long cube magnets with a 500 μm spacing. With a magnetic tweezer apparatus in this arrangement, the theoretical forces on the magnetic particle achieve a peak of 480 pN for 1 mm magnets, and 240 pN for 1 mm magnets. A single cylindrical axially magnetized magnet also can be used to generate a magnetic field gradient needed to induce a magnetic force on magnetic particle tagged polymers. In this case, the magnetic field orientation is radially symmetric around the center of the magnetic, so the magnetic particle is free to rotate. The magnetic field distribution along the center axis of a cylindrical magnet can be described explicitly, and can be used to estimate the magnetic forces on a 2.8 μm Dynabead magnetic particle. As shown in FIG. 6, the magnetic force can be tuned by varying the distance between the face of the cylindrical magnet and the magnetic particle.

These forces are sufficient to overcome the electrophoretic force acting to pull a DNA polymer into a protein nanopore reader, utilizing a voltage bias applied across the nanopore (estimated to be between 8 and 60 pN).⁷⁻⁸ In this scenario, where the polymer experiences a magnetic force pulling it out of the nanopore reader, and a simultaneous opposing electrophoretic force pulling it into the nanopore reader, the net force can be controlled by modulating either the magnetic force, the electrophoretic force, or both forces simultaneously. The magnetic force can be tuned (ideally in a range between 0.1 and 200 pN) by moving the distance between the magnet and the magnetic particle, as shown in FIG. 5. The electrophoretic force can be tuned by adjusting the applied voltage bias across the nanopore reader, where the electrophoretic force is described by Equation 2:

F_{elec}=ΔV/l·q Equation 2

where ΔV is the voltage drop across the nanopore reader, l is the length of the constriction zone of the nanopore reader, so that ΔV/l is the electric field within the nanopore reader constriction zone, and q is the electrical charge on the polymer within the constriction zone. Therefore, the electrophoretic force can be tuned by varying the voltage applied to the nanopore reader using external electrodes. In this case, and in the absence of motion which would introduce drag forces, the net force able to drive the polymer through the nanopore reader is described by Equation 3:

Fₙₑₜ=F_{elec}−F_{mag}=ΔV/l·q−½(Mₛm∇·)B Equation 3

The net force placed on the magnetic particle tagged protein governs the velocity of the polymer through the nanopore reader. When under force, the tagged polymer begins to accelerate and gain velocity. However, the moving polymer is subject to viscous drag forces due to interactions with the surround medium. These viscous drag forces increase with the polymer velocity, until the polymer reaches a terminal velocity, vₜ, where the drag forces and the net driving force offset each other:

Fₙₑₜ=−F_{drag}(vₜ) Equation 4

The drag force on the tagged polymer has two primary contributions: drag from the magnetic particle tag, which has a much larger surface area than the polymer, and drag/friction due to interactions between the polymer and the interior of the narrow protein nanopore reader:

F_{drag}=F_{drag,part}(vₜ)+F_{drag,reader}(vₜ) Equation 5

The drag on the particle can be estimated using Stoke's law, where the drag force on a spherical particle is determined by the solvent dynamic viscosity, μ, the velocity, v, and the particle radius R:

F_{drag,part}=6πμvR Equation 6

The drag forces from within the nanopore are more difficult to describe explicitly because they arise from complex electrostatic and chemical interactions between the polymer and the nanopore reader, in addition to viscous forces with water residing within the nanopore, but the reader drag force can be estimated by considering the translocation velocity observed for DNA (with no magnetic particle tether) within nanopores at typical voltage biases. At 120 mV applied potential, the force on ssDNA in an α-hemolysin nanopore is estimated to be ˜8 pN,⁷ and the translocation velocity is ˜1 nucleotide/μs, or ˜0.34 mm/s given a nucleotide length of 0.34 nm. This velocity represents the terminal velocity for DNA traveling through the nanopore under the applied electrophoretic force. In comparison, a 1.4 μm diameter particle with 8 pN of applied force would have a terminal velocity of ˜0.38 mm/s, which is quite similar to the translocation velocity of DNA. This analysis indicates that the drag arising from polymer interactions with the nanopore and the viscous drag on the magnetic particle both make significant contributions to the terminal translocation velocity of the polymer.

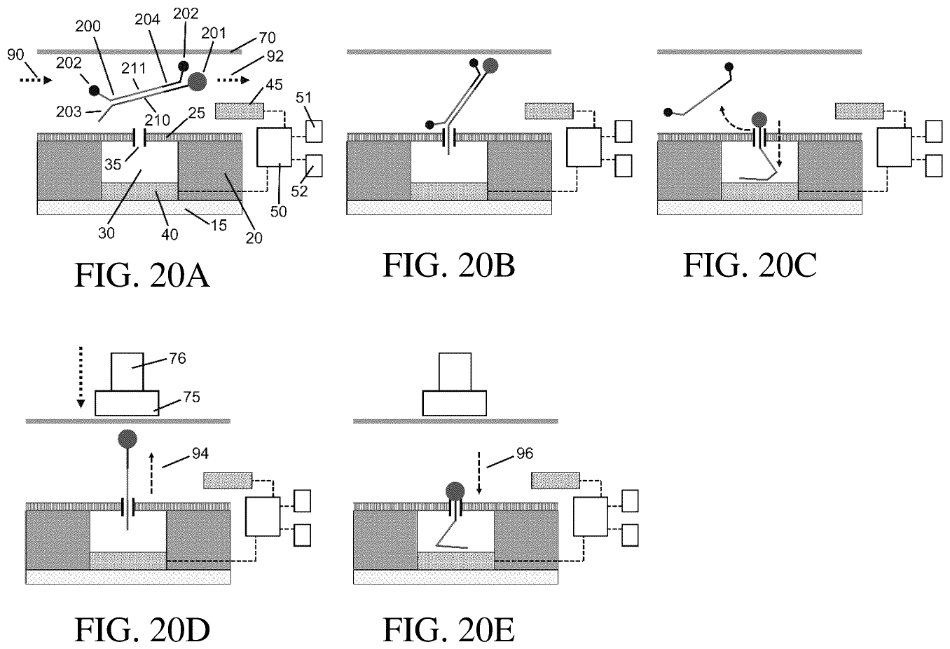

In systems that include a nanopore and a magnet and chamber each disposed on opposite sides of the nanopore, (i) a cis-to-trans direction often is towards the chamber and in a direction away from the magnet, and (ii) a trans-to-cis direction often is in a direction away from the chamber and towards the magnet. A trans-to-cis direction in a system is illustrated in FIGS. 20A-20E as element (94) and a cis-to-trans direction is illustrated as element (96). The voltage bias polarity applied to drive a polymer in a particular direction depends on placement of electrodes in a system and the net charge of the polymer being driven. In certain implementations, a negative voltage bias is applied to drive a polymer through a nanopore in a cis-to-trans direction, and a positive voltage bias is applied to drive a polymer through a nanopore in a trans-to-cis direction. In certain instances, a positive voltage bias is applied to drive a polymer through a nanopore in a cis-to-trans direction, and a negative voltage bias is applied to drive a polymer through a nanopore in a trans-to-cis direction.

### Opposing Magnetic and Electrophoretic Electroosmotic Forces Tension the Translocating Polymer

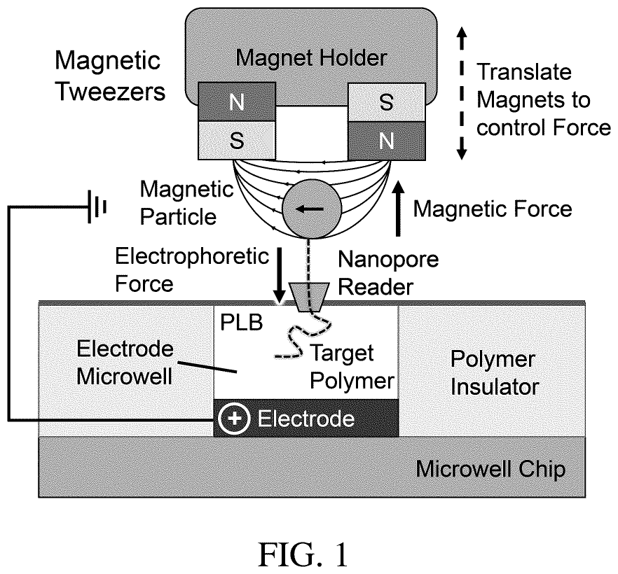

A requirement for nanopore sequencing is to hold the biopolymer under tension in order to reduce diffusion, disrupt secondary structure, and maximize the distance between monomers (bases or residues).⁹⁻¹⁰ For nanopore-based sequencing methods that use motor proteins and/or enzymes to controllably translocate a polymer through a nanopore, the target strand is held under tension between the motor protein and the strong electroosmotic and electrophoretic forces produced by the high electric field within reader's constriction or sensing zone. Rather than using a motor protein, magnetic tweezers,^{1, 11-12} are utilized to put the polymer under tension against the electrophoretic and electroosmotic forces that drive translocation. Magnetic tweezers apply force to a biomolecule tethered to a superparamagnetic particle interacting with an external magnetic field. After capturing a polymer in the reader, based on the inability of the magnetic particle to translocated through the reader, the magnetic tweezers can be moved into a set position to apply a constant force in opposition to the electrophoretic/electroosmotic force pulling the polymer into the reader. In certain instances, the direction of movement of the polymer can be controlled by varying the voltage bias that governs the electrophoretic/electroosmotic force (FIG. 1). By decreasing the voltage bias so that the electrophoretic/electroosmotic force on the polymer is lower than the magnetic force, the polymer can translocate up out of the reader while remaining fully extended between the reader constriction and the magnetic particle. The translocation direction can then be reversed by increasing the bias so that the electrophoretic and/or electroosmotic force is greater than the magnetic force. By changing the net force after each translocation, the polymer can be re-read as needed to increase consensus sequence accuracy on individual polymers.

In nanopore sequencing of polymers, the target polymer itself can be thought of as a one-dimensional “string.” When force is applied to each end of the polymer, the polymer is placed under tension, which is defined as a pulling force transmitted through the polymer. For polymers tagged with magnetic particles and placed in an external magnetic field, while simultaneously captured by the electrophoretic/electroosmotic forces within a protein nanopore reader, the forces applied to the polymer are described by Equation 1 and Equation 2, with the net force described by Equation 3. In addition to using this net force to control the translocation velocity of the polymer through the nanopore, the other primary objective of the magnetic force sequencing device is to use these opposing forces to apply tension on the polymer being sequenced.

Applying tension to the polymer serves two critical roles in nanopore sequencing. The first is that the applied tension elongates/stretches the polymer, within the sensing zone of the nanopore reader, which reduces its lateral diffusive movement and its tendency to form tightly coiled structures during translocation. Here, the tensile force as the magnitude of the magnetic and electrophoretic forces in Equations 1 and 2, F_{mag} and F_{elec} respectively, being applied to both ends of the polymer. When F_{mag} and F_{elec} are equal, there is no net force able to induce movement of the polymer. However, the polymer is still under a tensile force, Fₜₑₙₛ, that is able to stretch the polymer. If F_{mag} and F_{elec} are not equal, such that there is a net force on the polymer defined by Equation 3, then the polymer still experiences a tensile force equivalent in magnitude to the lesser of F_{mag} and F_{elec}. A net force on a polymer in a nanopore and a tensile force on the polymer can be determined from the magnetic force and the electrophoretic force. The net force and the tensile force on the polymer often are controlled independently and typically are considered separate forces.

Tensile force serves to elongate the polymer by overcoming the entropic tendency of the polymer to coil, or “ball up” by forming random bends and kinks between polymer subunits. These coiled structures interfere with nanopore based sequencing because coils and bends become caught in the nanopore constriction and interfere with translocation, much like trying to pull a tangled, knotted shoelace through an eyelet. In addition, the randomized orientation of the monomer units in coils add additional noise to monomer-dependent current signature, reducing the accuracy and precision of nanopore sequencing. The size of these coiled domains, d_{coil}, is governed by the ratio of the thermal energy of the system, and the tensile force on the polymer:¹³

d_{coil}=kT/Fₜₑₙₛ Equation 7
where k is Boltzmann's constant, and T is the temperature. For a real polymer, rather than an idealized flexible string, the size of these domains are limited by the actual dimensions of the monomer subunits in the polymer. At high Fₜₑₙₛ, the domains are smaller than the monomer length, and the polymer can be considered to be fully extended. For single-stranded DNA, the monomer subunit length can be modeled as the length of a single nucleotide, 3.4 Å. Using Equation 7, an estimated force needed to reduce dc oi to a size range smaller than the DNA subunit size is Fₜₑₙₛ=12 pN. In other words, a tensile force on the polymer>10 pN is needed to overcome the entropic coiling and fully elongate the polymer.

In addition to entropic coiling, polymers can also have strong attractive forces between monomer units that cause the polymer to fold into compact, thermodynamically favorable conformations. These compact, stable structures are referred to as polymer secondary and tertiary structure and are found in DNA, RNA, and proteins. Polymer secondary structure must be disrupted for the polymer to pass through the nanopore reader and be sequenced. One method to disrupt secondary structure is to use chaotropic agents or denaturants to alter the enthalpic interactions and entropy of the polymer and the surrounding solvent in order to make the unfolded state more thermodynamically favorable. This strategy can have downsides, however, as the denaturants can also disrupt the structure of the protein nanopore readers and PLBs necessary for sequencing. A better strategy is to “force denature” the polymers by pulling them apart with an applied tensile force. This method is routinely used to study intermolecular forces and energetics of biopolymers, such as DNA and proteins, using various force spectroscopies like AFM, optical tweezers, shear flow, and magnetic tweezers. With these methods, researchers have found that tensile forces in the 5-40 pN for are needed to disrupt various secondary structure motifs found in proteins, such as alpha helices, beta sheets, and the like.^{9-10, 12, 14} Similar forces are needed to disrupt structural motifs in nucleic acids; forces in the 5-15 pN range are needed to disrupt base pairing interactions,¹⁵ and forces between 10 and 45 pN are needed to disrupt G-quadruplex structures.¹⁶⁻¹⁷ These forces are well within the range that can be generated by magnetic tweezers (0.1-100 pN)² and the electrophoretic force produced by the protein nanopore (6-60 pN),⁷⁻⁸ indicating that the tensile force placed on the polymer by the reader and the magnetic particle can disrupt secondary structure to produce linearized polymers.

However, care must be taken to avoid applying too high a force to the polymer, which can have undesired effects such as breaking the polymer strand or disrupting the linker anchoring the polymer onto the magnetic particle. These unwanted disruptions to the polymer structure define the upper bound in the applied tensile force. For instance, forces in the 100-300 pN range have been shown to induce strand breaks in DNA.¹⁸ Although the streptavidin-biotin binding interaction that is commonly used to anchor the polymer to the magnetic particle is one of the strongest protein-ligand interactions, it can be disrupted at high tensile forces. The force needed to disrupt the biotin-streptavidin interaction varies over a wide range, from 40 to 200 pN, depending on the arrangement and direction of the applied force, the force loading rate, etc.¹⁹⁻²¹ For these reasons, care should be taken not to exceed a tensile force of approximately 60 pN.

Based on these considerations, it is desirable to apply a tensile force on the target polymer by applying an electrophoretic/electroosmotic force on one end of the polymer captured within the nanopore reader (this force is being applied on the polymer, within the sensing zone of the nanopore reader or within the reader itself), and a magnetic force on the opposite end of the polymer which is anchored to a magnetic particle, with the desired effect of extending the polymer. The tensile force generally is within a range where the force is high enough to extend the polymer and disrupt secondary structure, but not so high as to rupture chemical bonds and thereby break the polymer or detach the polymer from the magnetic particle. The applied tensile force required can vary between different polymer types (for example, DNA, RNA, and proteins), and for different nanopore readers (for example, α-hemolysin, MspA, aerolysin, CsgG). A typical tensile force used to stretch the polymer is about 2 pN to about 60 pN (for example, about 20 pN to about 50 pN, about 30 pN to about 50 pN, about 35 pN to about 55 pN or about 40 pN), however, a force of about 0.1 pN to about 200 pN can be employed (for example, about 0.1 pN, 0.5 pN, 1 pN, 2 pN, 3 pN, 5 pN, 8 pN, 10 pN, 12 pN, 15 pN, 20 pN, 25 pN, 30 pN, 35 pN, 40 pN, 45 pN, 50 pN, 60 pN, 70 pN, 80 pN, 100 pN, or 200 pN).

### Magnetic Tweezer Configuration

In alternative embodiments, provided is an apparatus designed to generate a magnetic field gradient that induces a magnetophoretic force on the magnetic particle-tagged polymers designed as a magnetic tweezer apparatus. The magnetic tweezers can include a magnet placed in a magnet holder. In alternative embodiments, the magnet holder is or is not attached to a fixed mount. In alternative embodiments, the magnet holder is or is not attached to a moveable mount able to change the distance between the magnetic tweezers and the sample. In alternative embodiments, the magnet is disposed in magnetic field proximity to the nanopore, sometimes in a fixed mount or sometimes at a position in a moveable mount.

In alternative embodiments, the source of the external magnetic field in magnetic tweezers includes a single permanent magnet (composed of a neodymium alloy, or other appropriate ferromagnetic materials), pairs of permanent magnets, arrays of permanent magnets, or electromagnets, as shown in FIG. 3. In the magnetic tweezers that utilize permanent magnets, the individual magnets can be used in various shapes, including cubes, rectangular prisms, cylinders, spheres, or have irregular shapes and pointed tips for edges. The size of the permanent magnets can vary in lateral width between 0.1 and 20 mm (for example, 0.1 mm, 0.5 mm, 1 mm, 2 mm, 3 mm, 5 mm, 10 mm, 20 mm), with height of 0.1 to 50 mm (for example, 0.1 mm, 0.5 mm, 1 mm, 2 mm, 3 mm, 5 mm, 10 mm, 20 mm, 50 mm). Pairs of magnets or arrays of magnets can include magnets having dimensions described above, arranged with gaps in between them. These gaps can be the same between all individual magnets or can vary across the array. Gaps between magnets can be between 0.05 mm and 20 mm (0.05 mm, 0.1 mm, 0.2 mm, 0.4 mm, 0.5 mm, 0.6 mm, 1.0 mm, 2.0 mm, 5 mm, 10 mm, 15 mm, 20 mm). Magnetic tweezers (composed of permanent or electromagnets) can include structures designed to focus or enhance the local magnetic field gradient in order to increase the force on the magnetic particle tagged polymers. These structures can be composed of materials with high magnetic susceptibility (for example, iron, nickel, mu-metal alloy) formed into thin strips, cones that taper to sharp points,^{4, 22} or tapered structures²³ with a small gap that approach the magnetic particle (FIG. 4).

In certain implementations, magnetic tweezers composed of permanent magnets (single magnets, pairs, or arrays of magnets) often are attached to a movable mount that can translate normal to the surface of the sensor chip to change the spacing between the magnetic tweezers and the magnetic particle tagged polymer, in order to control the force applied to the magnetic particles. A movable mount can be controlled with a manual threaded screw, a manual micrometer, or an electronically controlled motorized micrometer, linear actuator, piezoelectric actuator, and the like. The movable mount can have a travel distance between 1 mm and 100 mm (for example, 1 mm, 5 mm, 12 mm, 25 mm, 50 mm, 100 mm). A movable mount can also include a rotating component, either manual or motorized, optionally with electronic control, that is able to rotate magnetic tweezers including pairs or arrays of permanent magnets to alter the orientation of the magnetic field in order to rotate or induce a torque on magnetic particle tagged polymers.

Magnetic tweezers utilizing an electromagnet can incorporate electronic control of the current in the electromagnet to control the magnetic field magnitude and gradient and control the force on the magnetic particles shown in FIG. 3E. Magnetic tweezers composed of one or more electromagnets can also include a movable mount in order to position the magnetic tweezers close to the sample. Magnetic tweezers can be composed of one, two, four, or more independently controlled electromagnet elements arranged in dipole, quadrupole, etc. arrangements. The current applied to the electromagnet elements (a single or multiple electromagnet coils) can be manually controlled or electronically controlled by an electronic device (for example, an FPGA controller) responding to triggering events in the nanopore reader ionic current signal. Electromagnets with a single electromagnet coil or multiple electromagnet coils can be individually controlled by applying an alternating current signal, or a pulsed direct current signal to produce a magnetic field distribution with controlled spatial distribution or time variance that is able to finely control the force on the magnetic particle tagged polymer in order to apply a rotational torque, oscillating force, or other motion.

As addressed herein, magnetic field proximity is a distance between the nanopore and magnet for which a portion of the magnetic field exerted by the magnet overlaps with the nanopore. In certain implementations, the distance between the bottom edge of the magnet or magnet array (for example, trans terminus of the magnet or magnet array) is about 50 micrometers (μm) to about 25 millimeters (mm) from the nanopore (for example, cis terminus of nanopore reader), and sometimes the distance is about 150 μm to about 1000 μm from the nanopore (for example, a distance of about 150 μm, 200 μm, 250 μm, 300 μm, 400 μm, 500 μm, 750 μm, 1000 μm, 2 mm, 3 mm, 5 mm, 7.5 mm, 10 mm, 20 mm, or 25 mm).

### Chemical Ligation of Polymers to Magnetic Particles

The term “magnetic particles” generally refers to solid particles containing a superparamagnetic material. In certain instances, superparamagnetic materials are nanoparticles of 1-20 nm in size which are composed of ferrimagnetic or ferromagnetic materials. At these small sizes, the particles have weak magnetic coercivity and thus to not exhibit a net dipole moment, but they show high magnetic susceptibility and adopt a magnetic dipole moment when exposed to an external magnetic field. These materials include ferromagnetic metals, such as iron, nickel, cobalt, or alloys of any of these metals, or alloys of ferromagnetic metals with non-ferromagnetic metals, such as alloys of iron and platinum, cobalt and platinum, manganese and iron, and the like. In addition, these superparamagnetic materials can include ferrimagnetic metal oxides, such as iron oxides (for example, magnetite, Fe₃O₄, maghemite, Fe₂O₃), or mixed iron oxides (for example, MnFe₂O₄, CoFe₂O₄, Zn_{0.4}Fe_{2.6}O₄). Magnetite often is utilized.

A single superparamagnetic nanoparticle can be used. A larger particle often is used, which sometimes is composed of an inert matrix material (including polystyrene, gold, or silica) in which multiple superparamagnetic nanoparticles are embedded. In this configuration, each larger particle typically contains a larger mass of superparamagnetic material, which increases the total magnetization of the particle, producing a large force in an external magnetic field gradient. These composite magnetic particles often are spherical or substantially spherical in shape, and can be produced in varying sizes, ranging from 200 nm to 10 μm in diameter, including 200 nm, 500 nm, 800 nm, 1000 nm, 1200 nm, 2 μm, 2.8 μm, 3 μm, 5 μm, or 10 μm. In certain implementations, 1 μm and/or 2.8 μm particles are used.

Magnetic particles generally undergo further surface modification to allow polymers targeted for sequencing to be immobilized onto the particle surface. Surface modification can include addition of reactive chemical tags, or affinity tags such as streptavidin or antibodies which can be ligated to target polymers. In the case of silica magnetic particles, anchoring tags can be introduced to the surface via silane modification, or deposition of polyelectrolyte layers with chemical modifications. Polystyrene magnetic particles are typically synthesized via dispersion polymerization with a mixture of styrene and a co-monomer containing a carboxylic acid group. This method produces carboxylate groups on the surface of the particles which can be targeted for ligation with other reactive groups (for example, alkyne, azide, thiol, maleimide, and the like) or affinity tags (for example, streptavidin, antibodies) that contain a primary amine using carbodiimide amine-carboxylate linker chemistry. In some embodiments, magnetic particles can be further modified with polymers designed to passivate the surface in order to reduce nonspecific adsorption of polymers or interactions with lipid bilayers. Passivation polymers can include polyethylene glycol, polyacrylate, polysaccharides, or short polypeptides.

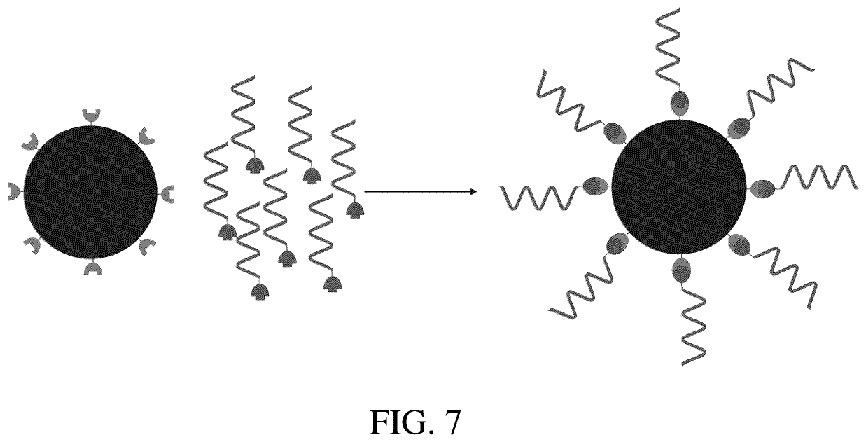

One straightforward way to attach a polymer to a magnetic particle is by using streptavidin coated magnetic nanoparticles and biotin tagged polymers (i.e., polymers that contain a biotin molecule at one of their ends, either 3′ or 5′ for DNA and RNA, or on their N or C terminus for a protein or peptide), as depicted in FIG. 7. Alternatively, polymers tagged with antigens, such as digoxin, may be attached to magnetic particles containing antigen-binding proteins, such as antibodies (anti-digoxin), Fab fragments, or nanobodies immobilized onto the magnetic particle surface. In the case of nucleic acids, any suitable length or sequence of either DNA or RNA oligonucleotides can be immobilized onto the particles and hybridized with a complementary DNA or RNA sequence attached to the polymer to immobilize the polymer onto the magnetic particle. The size of the particles can be from 50 nm diameter to 50 um diameter. Other functional group chemistries that can be used on either the particles, the polymers, or both include but are not limited to: carboxyl/amine, NHS/amine, Maleimide/thiol, thiol/thiol, amine/aldehyde, hydroxylamine/aldehyde, alkyne/azide, SNAP-Tag/benzylguanine, Clip-Tag/O₂-benzylcytosine, ACP/CoA, MCP/CoA, and diene/dienophile. In the previous examples, either group of a functional group pair can go on the beads or the polymer. To attach the polymer to the beads, beads can be washed, placed in a suitable buffer and incubated with the tagged oligonucleotides for the required time based on the reactive groups. Following sufficient incubation, the beads can be washed multiple times to remove any excess or unbound polymers, then reconstituted in a suitable buffer, where the tagged polymers are driven into a nanopore reader and controllably sequenced.

In certain implementations, one polymer is attached to an individual magnetic particle. In certain instances, two or more polymers are attached to an individual magnetic particle, even though only one of the polymers attached is sequenced by a nanopore reader at a time. In certain implementations, about two polymers to about 10,000 polymers (for example, about 1,000, or 2,000, or 10,000 polymers) are attached to an individual magnetic particle, even though only one of the polymers attached is sequenced by a nanopore reader at a time. In certain instances, 1 to about 10, or 1 to about 100, or 1 to about 1,000 polymers are attached to a magnetic particle.

A nucleic acid, such as a target nucleic acid polymer or oligonucleotide described herein, can be single stranded or double stranded, and can be DNA, RNA or a nucleic acid analog that can contain one or more nucleotide analogs. A nucleic acid analog or nucleotide analog can include a modified backbone, sugar and/or nucleobase, relative to DNA or RNA. Non-limiting examples of nucleic acid analogs include peptide nucleic acid (PNA), bridged nucleic acid (BNA), morpholino and locked nucleic acid (LNA), glycol nucleic acid (GNA), threose nucleic acid (TNA), hexitol nucleic acid (HNA), 2′-O-methyl-substituted RNA. Non-limiting examples of nucleotide analogs include inosine, thiouridine, pseudouridine, dihydrouridine, queuosine, wyosine, dideoxynucleotides, bromo-substituted nucleotides (for example, 5-bromouracil), deaminated nucleotides (for example, hypoxanthine) and fluorescent base analogs (for example, 2-aminopurine (2-AP), 3-MI, 6-MI, 6-MAP, pyrrolo-dC, 1,3-Diaza-2-oxophenothiazine, 1,3-diaza-2-oxophenoxazine). Non-limiting examples of modified nucleobases include diaminopurine, isoguanine, isocytosine, diaminopyrimidine, xanthine, 2-amino-6-(2-thienyl)purine, pyrrole-2-carbaldehyde and pyridine-2,6-dicarboxylate.

Target nucleic acids (also referred to as input nucleic acids herein) may in certain instances be attached to magnetic particles through hybridization interactions between, for example, oligonucleotides immobilized onto the magnetic particle and a complementary sequence on the nucleic acid or a complementary sequence tag ligated to the target nucleic acid. Alternatively, target genomic DNA can be hybridized to a complementary oligonucleotide tag immobilized on the magnetic particle, and then chemically ligated to the oligonucleotide tag to attach the genomic DNA to the magnetic particle. In certain embodiments, doubler or trebler phosphoramidites can be added to the DNA. The doubler/trebler phosphoramidites can have two/three of the same reactive or affinity functional group (for example, including, but not limited to biotin, amine, thiol, maleimide, carboxyl, NHS, aldehyde, hydroxylamine, alkyne, azide, benzylguanine, 02-benzylcytosine, diene, dienophile and the like) that can result in stronger immobilization of the DNA (or polymer) on the magnetic beads, at a lower density. Alternatively, multiple instances of these functional groups, which may include 2, 3, 4, 5, 6, or 10 instances, may be installed one after another in series on successive units of the polymer, or with an inert spacer group in between the reactive units, without the use of a branching doubler or trebler.

### Polymers for Sequencing

In alternative embodiments, sample polymers (for example, nucleic acid (for example, DNA, RNA), polypeptide) are modified to generate a conjugate that includes a magnetic particle at one end and a terminal capture polymer at the other end. A sample polymer (for example, a native polymer from a biological sample) sometimes is processed to generate an input nucleic acid that is modified to include the magnetic particle and terminal capture polymer, and sometimes a native polymer from a sample serves as the input polymer. A native polymer can be processed by any suitable process (for example, amplification, reverse transcription and/or fragmentation in the case of a native nucleic acid) to generate input polymer that is modified. A polymer also can be a modified non-native polymer. For example, modified DNA or RNA can include a modified backbone (for example, sugar modifications, linkage modifications) and/or modified bases), and a modified polypeptide can include a modified backbone, modified amino acids, peptidomimetic elements and/or modified side groups.

### Long Double-Strand DNA Capture

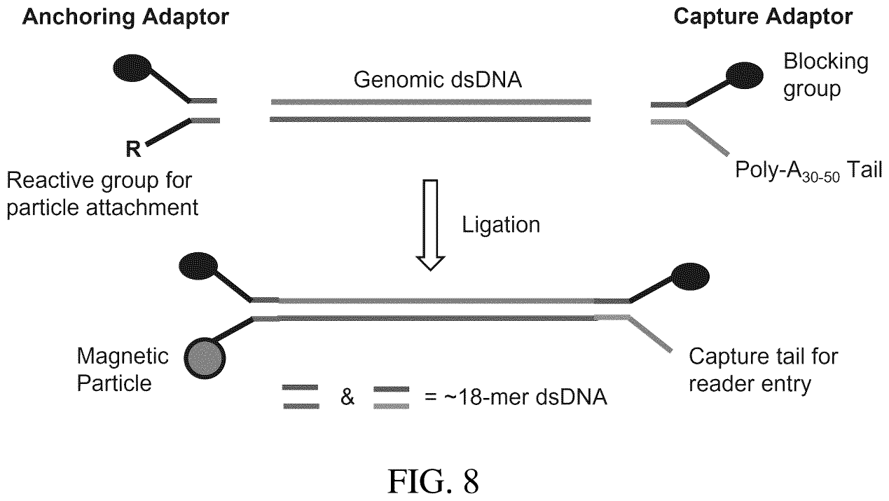

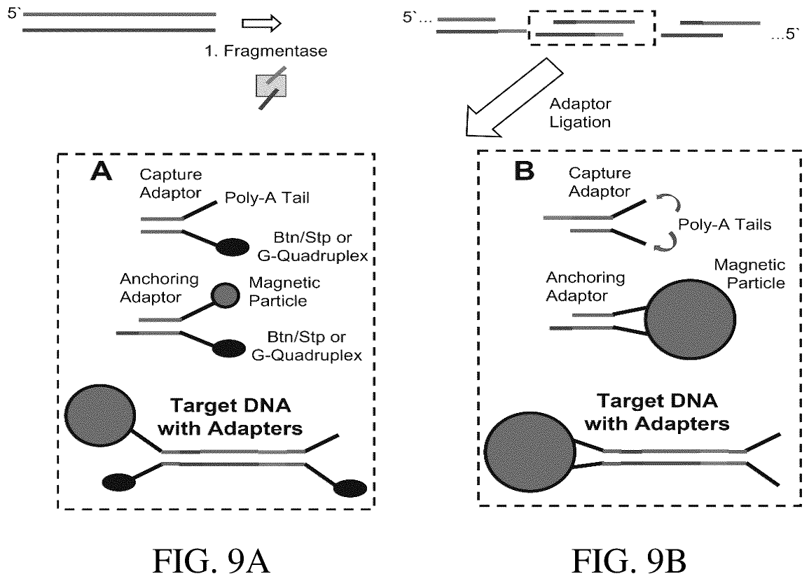

In alternative embodiments, one individual strand of double-stranded DNA is captured and sequenced in a magnetic sequencing system. One of the strands of the double-stranded DNA molecule can be captured by the nanopore reader and sequenced, while the other complementary strand can be pulled off and released from the target strand. In such implementations, the target strand can be conjugated to a single magnetic particle at one of its ends, i.e., 3′ to 5′, and a single-stranded overhang tail, that can be initially captured and threaded into the nanopore reader, can be conjugated to the opposite end of the target strand. In a generalized scenario, a magnetic particle and a polymer strand (for example, a ssDNA homopolymer of poly-A or poly-C, although any unstructured ssDNA sequence would be sufficient) are installed on opposite ends of the same strand of dsDNA (FIG. 8). In certain implementations, incorporation of adapters containing the single-stranded tail needed to thread one end into a nanopore and the magnetic particle to control the DNA motion through the sensor can be achieved by ligating Y-shaped adaptors comprised of dsDNA with two different sets of ssDNA tails onto the dsDNA target using standard library preparation protocols (blunt end ligation, sticky end ligation, TA cloning, or chemical ligation) (FIG. 9). Two types of adapters can be employed in certain implementations: a “capture” adapter which contains a polyelectrolyte polymer tail (for example, ssDNA) that is able to be captured and drawn into the nanopore reader via electrophoretic forces near its opening, and an “anchoring” adapter, which contains reactive groups or affinity tags needed to immobilize the DNA or RNA onto the magnetic particle. Once the modified DNA is captured, the complementary strand to the target strand being sequenced can be completely unzipped (via voltage induced translocation), leaving the ssDNA held taught/linearized across the reader via the opposing electrophoretic and magnetic forces to enable sequencing reads to be obtained.

In certain implementations, to ensure only one end of the target strand has accessible poly-A tails, and the other end has the magnetic particles for the desired capture orientation of that strand, a fragmentase digestion with dual custom synthetic 5′ adaptors can be used. Fragmentase digestion can install known sequences that function as handles for secondary ligation to install the free poly-A tail(s) on one strand (the poly-A tail is synthesized on the 5′ end) and the magnetic particle on the other strand. The fragmentase approach is traditionally used for small read sequencing; however, fragments greater than 2000 bp in length can be generated by decreasing the reaction time and fragmentase concentration.²⁴ This approach can facilitate working with real-world samples with unknown sequences. In certain implementations, Y-shaped adaptors ligated to the target DNA after fragmentase digestion can be designed in two different ways. In one way, one of the free poly-A tail ssDNAs enters the reader, the other tail forming the Y-shaped adaptor can be blocked from entry (for example, biotin-streptavidin complex, or bulky and highly stable G-quadruplex), and the duplex can be forced to unzip on the outside of the reader. In this scheme, the particle adapter on the opposite end of the target DNA strand can place a magnetic particle on the same strand as the poly-A tail used for threading, while the other tail can utilize a similar blocking approach to prevent entry into the nanopore (FIG. 9A). In the second approach, one of the ends of the dsDNA can include two, non-complementary poly-A ssDNAs, whereas the other end can have the magnetic particle attached to both ssDNA tails of the Y-shaped adaptor (FIG. 9B). In this scenario, either strand in the duplex can be captured and sequenced by the nanopore reader.

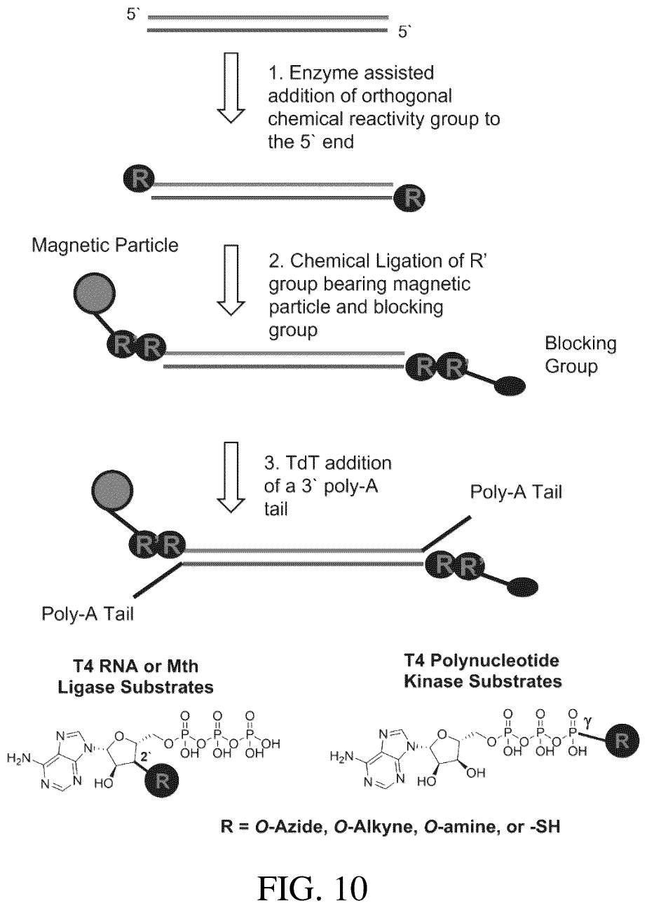

In certain instances, attachment of appropriate adaptors to dsDNA also can be achieved by a chemoenzymatic strategy (FIG. 10). In this approach, the 5′ ends of the target dsDNA can be modified with a chemical group that has orthogonal reactivity to those groups naturally found in DNA, which include but are not limited to phosphorothioates, primary amines, azides, or alkynes. These are added enzymatically in one of two ways in certain implementations: (1) a 2′-sugar modified adenosine triphosphate is added to the 5′ end of the 5′-phosphorylated dsDNA by T4 RNA ligase or Mth RNA ligase to yield a 5′ adenylated dsDNA with the chemical group on the 2′-O position; or (2) a γ-phosphate modified adenosine triphosphate can be allowed to react with the 5′ end of dephosphorylated target dsDNA via T4 polynucleotide kinase to add the γ-phosphate bearing the chemical handle to the 5′ end of the dsDNA target. The 5′ added orthogonal group can then be reacted with the appropriate iodoacetamide, dinitroflorobenzene, azide, or alkyne that is attached to the magnetic particle via a long ssDNA handle or other polymer to place the particle on all 5′ ends. Lastly, all 3′ ends can be reacted with terminal transferase (TdT) to add a poly-A tail that facilitates nanopore entry. In certain instances, to prevent ligation of magnetic particles to both 5′ groups on the target and complementary strands, these reactions can be carried out in a mixture of two 5′-modifier reagents: one that carries a reactive group for attachment to magnetic particles, and one that is inert and serves a blocking group to prevent magnetic particle ligation. This reaction mixture can produce a mixture of adapter-modified DNA strands, some of which have a single magnetic particle anchoring site, and some that have two sites, and some that have no sites. This mixture can be further separated to select the population with a single anchoring site using appropriate methods (for example, affinity chromatography). Alternatively, the reaction mixture can be tuned to have an excess of blocking reagent compared to anchoring reagent, so that the probability of producing adapted DNA with two anchoring sites is low. The DNA strands containing two blocking sites can be inert and can be removed using appropriate separation techniques, such as magnetic particle separation.

In certain implementations, an anchoring adapter used to attach the target dsDNA to the magnetic particle can be ligated to the target DNA and the magnetic particle in one of several ways. The anchoring adapter can contain a reactive group, R, that can be ligated to reactive groups, R′, on the magnetic particle to immobilize the adapter onto the magnetic particle. Alternatively, an adapter can contain a terminal affinity tag that binds to a ligand immobilized onto the magnetic particle (for example, biotin/streptavidin, digoxin/anti-digoxin, complementary oligonucleotides, and the like). The anchoring adapter can be first ligated to the target DNA, followed by immobilization onto the magnetic particle. Alternatively, the anchoring adapter can be first immobilized onto the magnetic particle, followed by ligation to the target dsDNA, in certain implementations.

### Long RNA Strand Adapters for Capture and Magnetic Particle Anchoring

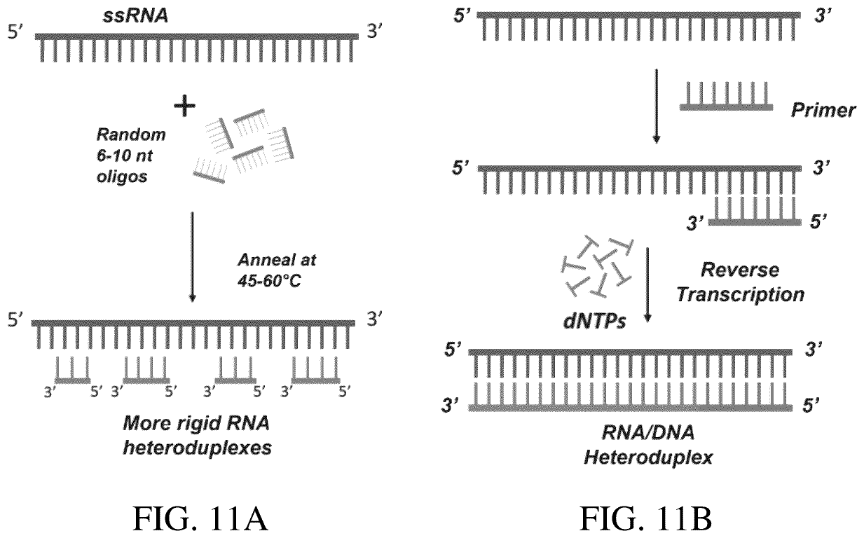

In alternative embodiments a magnetic sequencing system provided herein is used to sequence RNA. For instances in which the opposing electrophoretic and magnetic forces are not able to disrupt ssRNA secondary structure, i.e., a RNA molecule generally cannot simply be attached to a magnetic particle on one of its ends and then readily driven all the way through and captured via a nanopore reader, there exist at least two methods to circumvent this problem to allow sequencing. One approach is to disrupt and linearize ssRNA intramolecular structures and increase its persistence length using an excess of randomized DNA sequences (6-10 nucleotides in length) that can be annealed, thus creating a linearized, long-persistence length DNA:RNA heteroduplex (FIG. 11A). This also can be achieved using an excess of randomized RNA, 2′-O-methyl-RNA sequences, as well as nucleic acids in the form of locked (LNA), peptide (PNA), 2′-fluoro (FNA), or 2′-arabano (ANA) polymers. Similarly, a minimal library preparation protocol can be utilized, using reverse transcriptase to generate the complementary DNA strand (cDNA) to yield a cDNA:RNA heteroduplex (FIG. 11B). Heteroduplexes composed of short randomized annealed strands and continuous cDNA strands can be further modified with custom ssDNA strands to introduce a capture adapter, with a single-stranded tail for capture, and install the anchoring adapter needed for attachment to the magnetic particle. Both the capture adapter and the anchoring adapter typically are incorporated for sequencing.

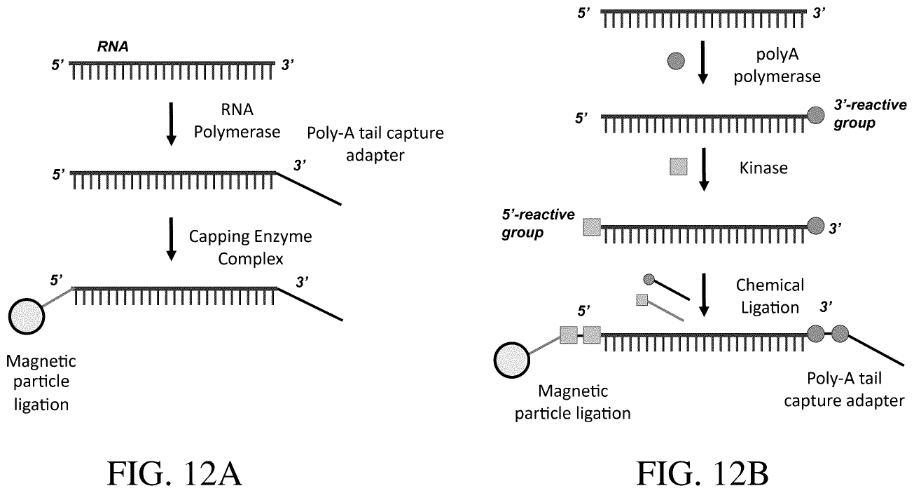

Strategies for tagging RNA with appropriate capture and anchoring adapters for sequencing can follow one of two general methods, in certain implementations. In a first general method, terminal groups can be utilized to selectively attach the capture adapter with the polymer tail to assist entry into the nanopore. This includes using or enzymatically installing the RNA 3′-poly-A tail as a capture adapter. In addition, alternative capture adapters or anchoring adapters can be installed via the 5′ cap on mRNA. One strategy is to oxidize the glycol present on the 5′-N7-methylguanosine cap with periodate to generate a bisaldehyde that can be used to ligate capture or anchoring adapters containing with aldehyde-specific reactive groups (for example, O-alkylhydroxylamines to yield oxime ethers, alkylamine addition to the aldehyde followed by reduction of the Shiff's base to yield a stable carbon-nitrogen attachment, aldehyde reaction with hydrazine to yield a hydrazone). Alternatively, mRNA and sense-stranded viral RNAs caps with NAD⁺ can be enzymatically functionalized with a clickable alkyne enzymatically with adenosine diphosphate-ribosyl cyclase²⁵. This clickable reactive group can be used to ligate either the capture or the anchoring adapter to the target RNA. Enzymatically incorporated poly-A tails and reactive caps can be used on all other target RNAs (for example, lncRNA, rRNA, tRNA, snoRNA, piwiRNA, and the like) (FIG. 12A). In a second general method, the 5′ and 3′ ends of the target RNA can be functionalized with an orthogonal reactive group (for example, poly-A polymerase insertion of a clickable adenine nucleotide on the 3′ end, and kinase insertion of a clickable phosphate on the 5′ end) (FIG. 12B). These reactive end groups can be reacted with polymers serving as capture and anchoring adapters that possess reactive partners to ensure that a single capture and anchoring adapter is ligated onto opposite ends of the same strand.

### Targeted Sequencing of Genomic Nucleic Acid

In alternative embodiments, a goal is to sequence a targeted region of genomic nucleic acid to identify clinically significant variants or single-nucleotide polymorphisms (SNPs), structural variants, repeat expansions, or epigenetic marks. In certain instances, a library preparation process can focus on and/or enrich the desired targeted regions. These regions of the genome can be identified, followed by excising a segment of the DNA containing the region of interest from the surrounding genome, and selectively ligating anchoring adapters and capture adapters to the targeted segment to enable sequencing with a magnetic sequencing system.

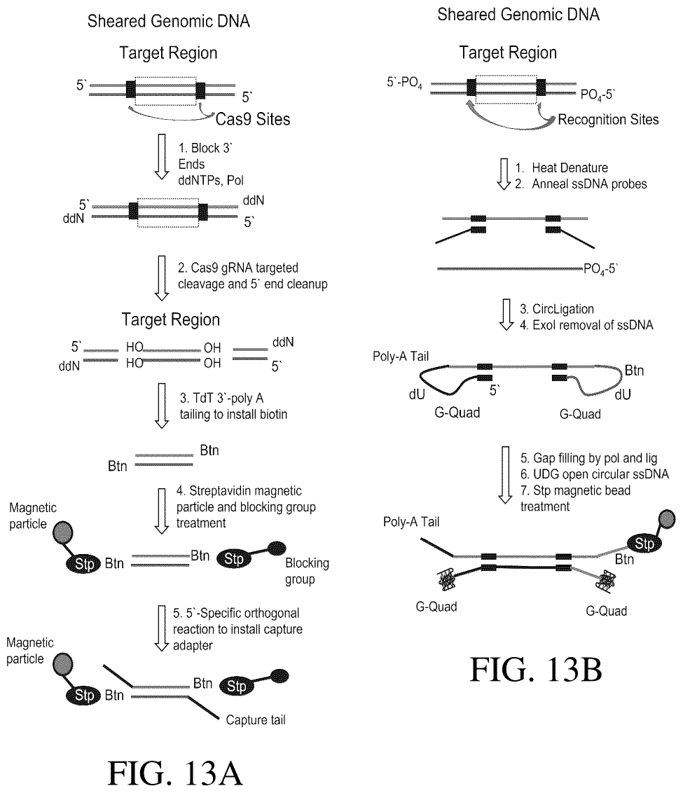

In certain implementations, input nucleic acid (for example, genomic DNA) can be sheared to an appropriate length distribution via a fragmentation process (for example, ultrasonication, restriction enzyme fragmentation, fragmentase digestion). Next, one of three different targeted approaches can be used in certain instances, some of which are modified adaptations from literature methods.²⁶⁻²⁷ In one approach, the 3′ ends of the genomic DNA (not near the region of interest) can be modified with blocking groups to prevent adapter ligation to these DNA fragments outside the region of interest. 3′ ends can be blocked using a DNA polymerase (for example, Klenow) with 2′,3′-dideoxynucleotides (ddNTPs). Next, the genome region of interest can be cut out using sequence-specific cleavage using custom guide RNAs (gRNAs) for Cas9-mediated cleavage of the DNA to expose reactive 3′ ends (FIG. 13A). These freshly exposed 3′ ends typically lack blocking groups, and can be reacted upon with TdT to form a poly-A tail in which a substoichiometric amount of 3′-biotinylated dATP is added to ultimately generate a 3′ end with a poly-A tail terminated with biotin. The 3′-biotinylated strand can serve as the mechanism for strand enrichment and serve as the anchoring adapter for immobilization onto streptavidin-coated magnetic particles, and capping the opposite end with a blocking group to prevent entry into the nanopore reader. The 5′ end can then be reacted upon via the orthogonal chemical reactivity group as described above (FIG. 10) to install a long homopolymer strand or other polymer for entry into the nanopore. Alternatively, the poly-A tail installed on the 3′ end can be used as the capture adapter, while a reactive group is installed on the 5′ end, serving as an anchoring adapter for ligation to the magnetic particle. This approach can be multiplexed using pairs of designer gRNAs to target multiple regions of interest in the genome.

In another approach, fragmented input nucleic acid (for example, sheared genomic DNA) can be targeted with ssDNA probes with long ssDNA regions designed to be complementary to regions near the targeted sequence of interest. These probes are designed to hybridize at the ends of the targeted sequence and serve as capture and anchoring adapters for sequencing as illustrated in FIG. 13B. To prepare the genomic DNA for modification with these hybridization adapters, the DNA can be heat denatured and annealed in the presence of the ssDNA probes. The annealed target sequence can be hybridized to ssDNA probes that contain two ssDNA tails that are substrates for circLigase for circularization of the target DNA ends. The remaining complementary genomic DNA strand can be removed by ExoI digestion. The added sequences can serve two roles that include use of primers to make a dsDNA with a long persistence length and the strands can have 2′-deoxyuridine nucleotides that can be cleaved to a stand break using a USER® system (New England Biolabs). After cleavage, the probes can place a poly-A tail on one end of the target sequence, serving as the capture adapter, and a base-biotinylated nucleotide on the opposite end, serving as an anchoring adapter for immobilization on streptavidin-coated magnetic particles for sample purification and enrichment, and sequencing. To ensure the complementary strand made by polymerization does not enter the nanopore, the cleaved ends of the probe strands retained on the complement can have a G-rich sequence that forms stable G-quadruplexes. These G-quadruplexes are too bulky to enter the nanopore, and they can serve as blocking groups that can prevent entry and assist unzipping of the complementary strand outside the channel. This method can be multiplexed using multiple designer probe strands that target multiple regions of the genome.

Another approach for targeted sequencing can include judicious selection of endonucleases designed to digest the genome at specific locations near the targeted region of interest. This endonuclease digestion typically yields two different sticky ends flanking the region of interest which can be ligated with capture and anchoring adaptors that contain complementary sticky ends. These adapters place the required functional groups on the strands needed for sequencing (FIG. 9).

### Modifying Polypeptides for Sequencing

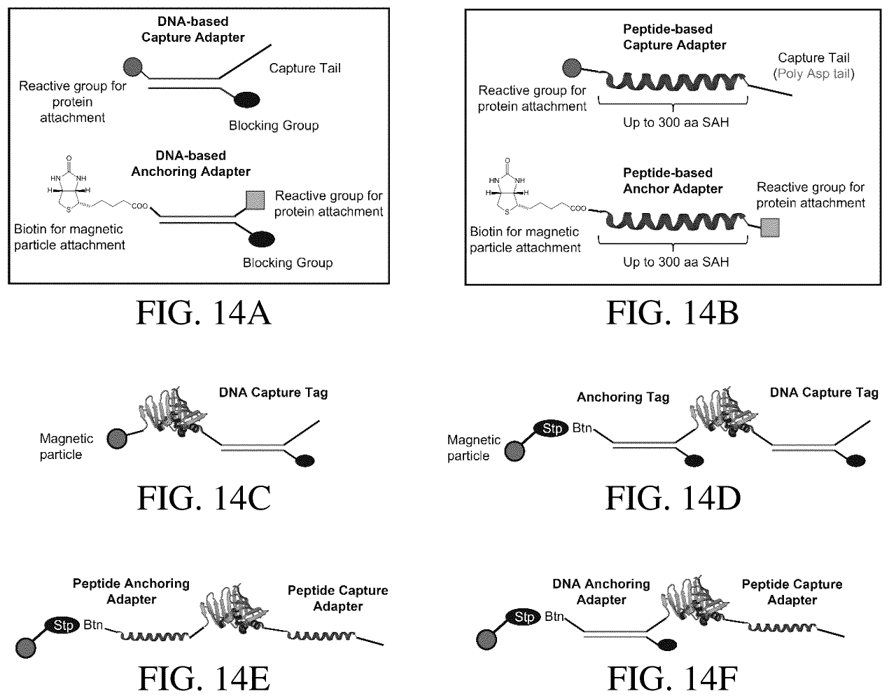

In alternative embodiments, in order to sequence a protein polymer using the magnetic force sequencing device, terminal adapters often are incorporated onto both the N- and C-termini of the protein for two reasons: (1) to add a capture tail that can electrophoretically drive the protein to and capture it in the reader, and (2) anchor the other end of the protein to a magnetic particle, such that the two tags/ends can be pulled in opposite directions to apply tension that denatures, elongates and stretches the protein into a nearly linear strand, readying it for controlled translocation and sequencing. One method of tagging the N or C termini of a protein is with a DNA “capture tag” although the capture tag can be any charged polymer (see below). The capture tags can be double-stranded/single-stranded DNA (ds-ssDNA) synthetic constructs (FIG. 14A) ligated to the C or N terminal ends of the proteins. The ssDNA ends of the adaptors can be comprised of unstructured heteropolymer or homopolymer DNA, about 20 to about 1000 consecutive nucleotides (nt) in length (often about 40 to about 100 consecutive nucleotides in length), for selective entry into the reader, and the other end of the DNA terminated in a maleimide or alternative reactive group for attachment to the protein. The ends of the complementary strand also can be unstructured DNA (unstructured refers to the sequence of the DNA to be attached being judiciously designed such that it does not adopt stable secondary structures such as hairpins, G-quadruplexes, H-DNA, or i-motifs), 20-1000 nt in length, or an unstructured charged polymer of similar length, terminated with a bulky and denaturant-resistant G-quadruplex forming DNA sequence that blocks this end from entering the reader.⁴ Other bulky macromolecules or nanoparticles blocking the complementary strand can be used, including without limitation biotin/streptavidin, dendrimers, polysaccharides, polyethylene glycol, gold nanoparticles, or polystyrene nanoparticles. The anchoring adapter can have a similar structure, with a ssDNA 20-1000 nt in length that has a terminal reactive group for attachment to the C- or N-terminus of the target protein, and an affinity tag on the opposite end for attachment to the magnetic particle. The complementary strand annealed to the anchoring adapter can also include a terminal blocking group to prevent entry into the nanopore reader and assist force-dissociation of the complementary strand during sequencing (FIG. 14A). DNA tags can be commercially synthesized by a combination of chemical and enzymatic steps to include a 5′-maleimide, NHS-ester, carboxy, peptide enzymatic ligation tag⁴, or other reactive functional groups for ligation to amino acids. The judicious design of the adaptor arms relative to the strand with the maleimide can ensure that only the protein-DNA covalently linked capture strand enters the readers and can facilitate mechanical unzipping of the blocked (by G-quadruplex or other bulky molecules) complementary strand. Longer variations and/or alternatives of these adapters can be utilized if necessary. Additionally, other rigid polymers with long persistence length, such as peptides that form single α-helices (SAH), can be used as tags for protein capture by a nanopore reader (FIG. 14B). These polypeptides can include other structural motifs including collagen-like helices and coil-coil structures. A SAH capture tag can include a repeating polypeptide sequence (for example, about 3,000 amino acids in length) that folds into a rigid α-helical structure, which has a long persistence length comparable to dsDNA (20-100 nm)⁵-6. SAH-forming peptides can be captured and translocated through nanopore readers much like single-stranded DNA.⁷⁻⁸ The terminal ends of the SAH peptides also can be modified with a flexible charged polypeptide, polymer, or ssDNA tail to enhance the capture rate by a nanopore reader. The distal end of the SAH peptide often contains a reactive functional group for ligation onto the C- or N-terminus of a target protein or polypeptide using methods described herein. In certain implementations, a peptide-based anchoring strand is utilized and contains a reactive group for attachment to the C- or N-terminus of the protein, and an affinity tag on the opposite end for anchoring to the magnetic particle (FIG. 14B).

The ability to perform site-specific modification of the termini of proteins is an active area of research. N-terminal specific modifications have been achieved by various methods, mainly exploiting the pKₐ differences between lysine (pKₐ˜10) and the N-terminal amine (pKₐ˜8),²⁸⁻³¹ with the N-terminal amine reacting via multiple methods including reductive alkylation,³² and installation of an azide by reaction with azidoacetic anhydride,³³ for example. Attachment to the C-terminus can be carried out by enzymatic attachment of affinity tags using carboxypeptidase Y,³⁴ as well as by the differences in oxidation potential between the C-terminus and glutamate/aspartate, for single electron transfer reactions to alkylate the C-terminus.³⁵⁻³⁷ Attachment of capture tags to the C-terminus also can be targeted to specific amino acid motifs³⁸ using sortase,³⁹ peptide asparaginyl ligase,⁴⁰ or other enzymatic methods.⁴¹

Because proteins and peptides do not have a polymer backbone with intrinsic charge like DNA and RNA, their net charge can vary significantly based on their amino acid content. The C- and N-termini of the protein or peptide can be ligated to a charged polymer to increase its charge density and facilitate electrophoretic capture and translocation through the reader. This polymer can be, but is not limited to a polypeptide (for example, polyarginine, polylysine, polyglutamate), a DNA polymer, an abasic DNA homopolymer, or a synthetic polymer (for example, polystyrene sulfonate, polyallylamine, polyacrylate, polyvinyl sulfonate). The length of a homopolymer can vary from about 5 to about 10,000 monomer units, for example. The length can be between about 100 and about 1,000 monomer units, with the length chosen to facilitate high ligation coupling yield and high capture rates by a nanopore reader. Alternatively, charged surfactants (for example, sodium dodecyl sulfate) can be added to the fluid to increase the charge density of the protein or peptide via adsorption to facilitate capture and translocation through a nanopore reader. Added surfactant also can serve to denature the protein or peptide secondary structure, facilitating unfolding, stretching, and translocation through the readers. Alternatively, or in addition to surfactants, other denaturants including but not limited to urea or guanidinium chloride can be added to the fluid to denature the protein or peptide secondary structure to facilitate unfolding, stretching, and translocation through the readers.

### Microwell Sensor Chips

In alternative embodiments a substrate is a base structure of a chip onto which a chip is built. A chip can have a single sensor site on it or thousands of sensor sites. One or more microwell sensors in a magnetic sequencing device are sometimes fabricated on a fused quartz/silica substrate that is approximately 0.5 mm thick, but other high-resistivity, low-loss substrates are also appropriate to limit parasitic capacitance (for example, glass, sapphire, printed circuit board laminates like polycarbonate or PTFE). The substrate thickness sometimes is about 0.1 mm to about 2.0 mm (for example, about 0.1 mm, 0.15 mm, 0.2 mm, 0.25 mm, 0.30 mm, 0.35 mm, 0.40 mm, 0.45 mm, 0.50 mm, 0.55 mm, 0.60 mm, 0.65 mm, 0.70 mm, 0.75 mm, 0.80 mm, 0.85 mm, 0.90 mm, 0.95 mm, 1.00 mm, 1.10 mm, 1.15 mm, 1.20 mm, 1.25 mm, 1.30 mm, 1.35 mm, 1.40 mm, 1.45 mm, 1.50 mm, 1.55 mm, 1.60 mm, 1.65 mm, 1.70 mm, 1.75 mm, 1.80 mm, 1.85 mm, 1.90 mm, 1.95 mm or 2.00 mm). The substrate then containing or holding/supporting the electrodes, contacts, interconnects, and insulative layer is referred to as a chip.

FIG. 1 shows a single sensor site on a chip, here also designated as a “well” or “microwell.” A sensor site typically includes an electrode. An electrode can include a metal such as Pt, Au, or Ag and can have an underlying adhesion layer such as Ti or Cr. An electrode can include a composition of a semiconductor electrode, such as indium tin oxide, can include a conductive polymer such as PEDOT, or can include metals and other materials stacked in multiple layers. One or multiple larger electrodes can be included in or on a chip to form one or more reference electrodes (for example, for the bath), or one or more COTS external reference electrodes can be utilized. Variants with multiple reference electrodes can include different metals such as one electrode Pt and the other Ag for example. The Ag electrode variant chips can be treated to form stable Ag/AgCl electrodes. Electrodes can be connected to contact pads on the chip, comprising Au or Pt on the periphery or backside of the chip to connect the chip to a measurement system. Through vias also can be used to connect the electrodes on one side of the chip to the contact pads on the other side of the chip.

A chip can be covered with an insulator, such as SU-8, polyimide, parylene, para-xylylene, parylene HT poly (P-Xylylene), parylene F, polystyrene, PTFE, CYTOP, PMMA, polycarbonate, epoxy, polydimethylsiloxane (PDMS), or fluoropolymers, and other coatings that meet the specific requirements of chemical compatibility, insulative, and low-loss that are acceptable. In some cases, an insulator can include a surface treatment such as an additional thin polymer coating, plasma (for example, SF₆, CHF₃, C₄F₈), silane, or other chemical treatment to achieve the appropriate surface properties. An insulator (for example, a polymer insulator) can have a thickness of about 0.1 micrometers (m) to about 500 micrometers (for example, a thickness of about 0.1 μm, 1 μm, 2 μm, 5 μm, 10 μm, 20 μm, 30 μm, 40 μm, 50 μm, 60 μm, 70 μm, 80 μm, 90 μm, 100 μm, 150 μm, 200 μm, 250 μm, 300 μm, or 500 μm). An insulative layer can be photopatterned, printed, molded, stamped, and/or etched in a manner to expose all the electrodes and contacts, thus creating openings over the electrodes and access to the contact pads. The openings can be then enlarged using thin-film processes, for example, etching or ion milling, such that for a given sensor the diameter (or maximum width) of the opening is about 1 micrometer to about 500 micrometers (for example, a diameter of about 1 μm, 2 μm, 5 μm, 10 μm, 20 μm, 30 μm, 50 μm, 70 μm, 100 μm, 200 μm or 500 μm).

In alternative embodiments the openings in a polymer layer often are designed to support a free-standing planar lipid bilayer (PLB), or other free-standing membrane composed of phospholipids, block copolymers, or surfactants. A PLB often serves as an ion impermeable membrane separating a cis portion and a trans portion of the device, and as a substrate for insertion of nanopore readers. The membranes are typically formed from a mixture of a hydrophobic solvent immiscible with the aqueous sample solution, and a membrane-forming material, such as phospholipids, block copolymers, and the like. Once formed, the PLB membranes often include a thick solvent-rich “annulus” structure around the perimeter of the opening that tapers laterally down to a thin bilayer membrane spanning the opening of the well. The openings in the polymer can be of any suitable geometry, and sometimes are circular in shape or ellipsoidal or polygonal in shape. The polymer surface around the openings can be smooth and flat, or it can contain nano- or micro-scale structures designed to improve the stability, lifetime, formation success rate of the annulus and membrane structure. These can include arrays of raised structures on the top surface of the polymer around the opening, including cylindrical pillars, rectangular pillars, pyramidal structures, or periodic undulations in the polymer height. The purpose of these structures is to trap a volume of the hydrophobic solvent/lipid mixture in a region around the opening. This prevents the solvent/lipid mixture from spreading over the surface and thinning, thus maintaining a reservoir of solvent/lipid material near the opening which maintains the stability of the annulus and improves the lifetime and stability of the membrane. Additionally, an outer perimeter around an opening in a polymer layer can include notches or grooves, which serve to capture and maintain a reservoir of hydrophobic solvent/lipid mixture around the opening to improve membrane stability. In addition, the polymer can include multiple concentric openings of varying diameter, the edges of which serve to act as reservoirs for the solvent/lipid mixture. The openings in the polymer can include these features in any combination, or include none of these features. These features can be implemented for retention of a hydrophobic solvent/lipid mixture at a well opening having a highly curved aqueous-solvent interface, such that the aqueous material in the trans portion forms a spheroid shape. In this configuration, the sensor wells contain spheroid droplets of aqueous sample that are able to form highly stable droplet interface bilayer membranes between the cis and trans portions. Such droplet structures are useful due to the stable, reproducible annulus and membrane structure which is formed in a highly reproducible manner.

### Nanopore Readers

In alternative embodiments, as used herein, the terms “nanopore,” “reader” and “nanopore reader” reference a structure and/or molecule containing a nanopore. Such a structure or molecule sometimes is a biological nanopore reader, an artificial engineered nanopore reader, a DNA nanopore reader, a peptide nanopore reader, a solid-state nanopore reader, or a nanopore reader containing a molecular adaptor.

In alternative embodiments, a biological nanopore reader utilized to sequence a single-stranded polymer (DNA or RNA), a double-stranded polymer (DNA or RNA), or a protein or peptide can be any suitable biological nanopore protein, ion channel, transmembrane protein or DNA nanopore suitable for strand sequencing applications. A biological nanopore reader can be a wild-type form (i.e., native form) of or a mutated, engineered, and/or chemically modified form. A sensing zone of a nanopore reader (for example, αHL or MspA) can be mutated to modify one or more of the following features: improve DNA translocation, increase or decrease electroosmotic flow through the nanopore under an applied potential, and have a single, sharp sensing zone that can resolve/sequence individual nucleotides or multiple nucleotides at the same time (i.e. a k-mer read frame). A reader with multiple sensing zones or reader heads also can be utilized. Mutations can also target the overall charge, size, electrostatics, and three-dimensional shape of the nanopore, or can be made to alter the interaction with a membrane (i.e., to more/less easily insert into a membrane, to remain inserted in the membrane longer). A more aggressive truncation or insertion of several amino acids can remove or introduce a recognition site, enhance sensitivity, and impart selectivity of a desired analyte. Mutations can also involve the introduction of non-natural amino acids with unique side chains and functional properties. Oligomeric protein or readers can be synthesized as a single chain. For instance, alpha-hemolysin, a heptamer, can have its seven subunits expressed as a single protein, with amino acid linkers introduced in between what were formerly separate subunits to connect them into a single chain that can fold into a functional nanopore. Various molecules can be added either through in vitro conjugation to the desired pore site or through fusion protein expression to enhance the nanopore performance.

In alternative embodiments completely synthetic biological based nanopores or ion channels also are suitable. Non-limiting examples of biological nanopores, ion channels, or transmembrane proteins that can be utilized as readers include alpha-hemolysin (αHL), aerolysin, Mycobacterium smegmatis porin A (MspA), Escherichia coli CsgG, Cytolysin A (ClyA), and outer membrane protein F (OmpF), NetB protein toxin, modified or mutant forms of secretin, and Fragaceatoxin C (FraC). Non-limiting examples of synthetic engineered biological nanopores include DNA nanopores (also referred to as “DNA-based nanopores” or “DNA origami nanopores”) and engineered peptide nanopores. Chemical crosslinking agents that covalently link individual subunits of a reader or that tune the performance of one or more readers also can be utilized.

In some instances, a nanopore reader can be utilized that yields a strong electroosmotic force through the nanopore, when an electric field is placed across the nanopore. In certain implementations, electroosmosis through the nanopore is in the same direction as electrophoresis such that both electroosmosis and electrophoresis pull a polymer, regardless of its charge or characteristics, into and through the nanopore, in opposition of the applied magnetic force on the magnetic particle on the end of the polymer. Electroosmosis is a net flow of liquid through a small diameter pore or capillary produced by the movement of counterions near the charged surface of the pore in an external electric field. As the counter ions move along the surface of the pore, they drag the viscous surrounding solvent with them, producing flow that can induce a force and directional migration onto molecules irrespective of the charge of the molecules. This configuration is suited for controlling the translocation and sequencing of proteins or peptides, because they can be neutrally charged or have a varying charge distribution, such that electrophoresis alone cannot be utilized to counterbalance the applied magnetic force on the polymer within the nanopore. Nanopore readers can be mutated or modified to contain additional charged residues that serve to alter the charge distribution on and within the nanopore reader to control the magnitude and direction of electroosmotic flow. For example αHL can be modified with additional positive charged residues (K and R) throughout its □-barrels (for example, T115K, T117R, G119K, and N121R) at positions that have been previously altered in order to enhance molecular transport through the pore.⁴²⁻⁴³ Similar modifications can be made in MspA: N79R, N86R, N108K or N121K in MspA. The cationic surface on the interior of the protein nanopore reader generates an excess of anionic counter ions within the pore, facilitating net flow into the interior of the pore under a positive applied voltage bias (trans to cis). Such modifications can drive electroosmosis in the same direction as electrophoresis (of the anionic target molecules) to and through the reader, with electroosmosis as the dominating force within the reader, allowing heterogeneously charged polymers, including neutral proteins and peptides, to be pulled in the direction desired. Electroosmosis has previously been shown to dominate over electrophoresis within nanopore confinements under controlled conditions with surface charge, pH, and electrolyte type and concentration, serving as the governing parameters for controlling the force balance.⁴⁴⁻⁴⁶ In certain instances, a salt gradient is applied to help drive a polymer into or out of a nanopore reader.

In alternative embodiments a nanopore of a nanopore reader sometimes has a maximum width or diameter of about 0.1 nanometers (nm) to about 500 nm (for example, about 0.5 nm to about 50 nm, about 1 nm to about 25 nm, about 0.5 nm to about 2 nm, or about 0.1 nm, 0.2 nm, 0.3 nm, 0.4 nm, 0.5 nm, 0.6 nm, 0.7 nm, 0.8 nm, 0.9 nm, 1 nm, 1.5 nm, 2 nm, 3 nm, 4 nm, 5 nm, 6 nm, 7 nm, 8 nm, 9 nm, 10 nm, 20 nm, 30 nm, 40 nm, 50 nm, 60 nm, 70 nm, 80 nm, 90 nm, 100 nm, 150 nm, 200 nm, 250 nm, 300 nm, 350 nm, 400 nm, 450 nm or 500 nm). The maximum width of a nanopore of a nanopore reader typically is less than the minimum width or diameter of a magnetic particle, and typically is less than the minimum width or diameter of a blocking moiety, of a polymer conjugate that is subject to sequencing in a magnetic sequencing system.

### Preparation of Magnetic Sequencing System

In alternative embodiments of a process for preparing a magnetic sequencing system for sequencing, a nanopore reader is associated with each well of a chip (for example, single well or multi-well chip). A reader can be associated with each well of a chip using the following non-limiting implementation. A chip often is initially bathed within an electrolyte solution or bath. A phospholipid bilayer (PLB) or other membrane often is then formed over each sensor well on the chip, using known methods to paint or cast thin films of membrane-forming materials over the well. Individual nanopore readers utilized then often are inserted into each target PLB, utilizing a voltage cycling process (see U.S. Pat. No. 8,968,539). Any suitable biological nanopore reader can be used for sequencing, for example, αHL, MspA, or CsG pores can be utilized, with typically one reader, in one PLB, over and/or spanning one well on the sequencing chip. A nanopore reader can be a wild-type biological reader, or another mutated, engineered, and/or chemically modified reader. The outer region of the chip where samples are introduced is referred to as the “cis” portion, or cis side of the reader, while the region on the opposite side of the membrane, in the interior of the sensor well is referred to as the “trans” portion, or trans side of the reader. Polymers typically translocate or are captured within the nanopore reader from the cis to trans portion, and sometimes polymers can additionally translocate from the trans to cis portion during multi-passing.

A process for constructing a biological nanopore reader device can include in certain implementations: forming photopatterned microwells into SU-8 photoresist with Ag/AgCl electrodes at the bottom of each well (for example, wells can be in a SU-8 layer that is about 1 m to about 200 m deep, with the bottom of the well backfilled with Ag/AgCl), forming a planar supported lipid bilayer over each well using a magnetic stir bar, forming a PLB (for example, by lipid painting or single-sided membrane formation), and application of a high voltage bias (100-300 mV) for insertion of a nanopore reader into one or more PLBs. While a PLB is referred to here over and/or spanning each well, any suitable membrane seal capable of retaining an individual reader can be utilized, including but not limited to a seal comprising phospholipids (for example, DPhPC, POPC, DOPC, DMPC, DoPhPC), surfactants, di-block copolymers (for example, polybutadiene—polyethylene oxide), tri-block copolymers (for example, poly-2-methyl-2-oxazoline—polydimethylsiloxane—poly-2-methyl-2-oxazoline), or polymerizable versions thereof.

Modification of Magnetic Particles and/or Membranes to Prevent Nonspecific Adsorption

In alternative embodiments, when a target polymer attached to a magnetic particle is captured by a nanopore reader inserted in a membrane, the surface of the magnetic particle approaches the surface of the membrane at a distance comparable to the length of the target polymer (typically 1 to 1000 nanometers in length). In alternative embodiments, a membrane is a bilayer membrane, lipid membrane, black lipid membrane or block copolymer membrane (for example, diblock copolymer membrane, dual block copolymer membrane, triblock copolymer membrane), sometimes includes one or more types of amphiphilic molecules, optionally includes one or more types of polypeptides, sometimes includes one or more types of fatty acids, optionally includes one or more types of polymers (for example, block copolymers (for example, diblock copolymers, dual block copolymers, triblock copolymers)), optionally includes one or more types of surfactants, optionally includes one or more types of lipids (for example, phospholipids), and optionally is a planar lipid bilayer (PLB).

In alternative embodiments, when the magnetic particle and the PLB are in close proximity, interactions between their respective surfaces may result in nonspecific adsorption of the particle to the PLB surface. This undesirable adsorption to the PLB may result in the particle being permanently “stuck” to the PLB, preventing the use of a magnetophoretic force to pull the magnetic particle attached to the target polymer away from the PLB, and thus preventing magnetic field from inducing tension and motion onto the polymer. These nonspecific adsorption interactions can result from electrostatic, chemical, van der Waals, or dipole-dipole interactions between chemical functional groups on the surface of the PLB and magnetic particle, respectively. Adsorption may also occur due to penetration of the magnetic particle into hydrophobic solvents used to coat the chip surface and help support the PLB. In order to prevent this undesirable nonspecific adsorption, surfaces of elements of the system, such as the magnetic particle, layer and/or the membrane, for example, may be chemically modified to block or reduce attractive interactions and thus mitigate or eliminate nonspecific adsorption. In certain implementations, after a membrane is formed over a layer at a sensor site and a nanopore is disposed in the membrane, a system can be treated with a passivating agent that modifies the membrane and/or layer. The magnetic particle of a conjugate may be treated with a passivating agent separately or in the same treatment used to treat the system containing the membrane and layer.

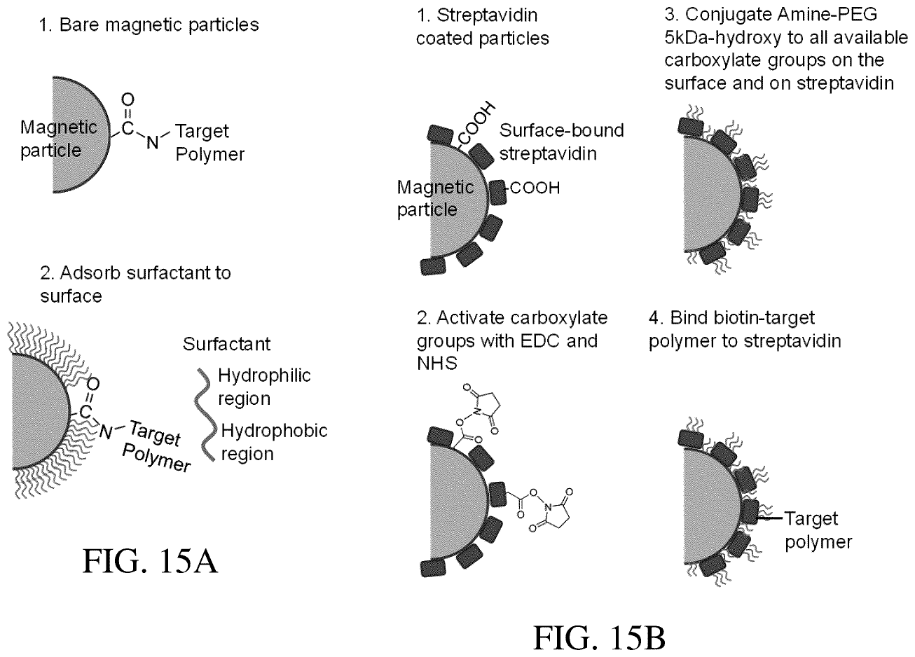

The surface of the magnetic particle may be modified by a surface-active passivating agent that is able to adsorb to the magnetic particle and alter its surface properties to prevent nonspecific adsorption (FIG. 15A). A surface-active passivating agent typically does not covalently attach to a magnetic particle and typically is not associated with a magnetic particle via an affinity tag. A surface-active passivating agent may include a surfactant, which is able to adsorb to the particle surface and alter its charge, thus preventing electrostatic attraction with the PLB, or alter the chemical functionality of the surface to prevent adsorption. Non-limiting examples of surfactants include detergents and polymers, such as sodium dodecyl sulfate (SDS), Tween-20, Triton X-100, octyl glucoside, digitonin, phospholipids, and pluronic block-copolymers. The particle may be similarly passivated by treating the surface with a protein known to coat surfaces and prevent nonspecific adsorption, such as bovine serum albumin (BSA). Particle surfaces may be coated with a surface-active passivating agent (for example, surfactant or protein) by incubating the particles with the passivating agent for a period of time. For example, BSA at a concentration of about 0.1 μM to about 1000 μM, or about 10 μM to about 500 μM, or sometimes a concentration of about 10 μM, 50 μM, 100 μM, 500 μM, or 1000 μM, can be incubated with particles for a time period of 1 minute to 24 hours (for example, 5 minutes, 10 minutes, 20 minutes, 30 minutes, or 60 minutes). After incubating the particles with a surface-active passivating agent, particles often are rinsed to remove excess passivating agent solution before use. During a capture and sequencing experiment performed with the magnetic particles, a concentration of surface-active passivating agent can be included in the experimental solution in order to prevent spontaneous desorption of the passivating agent from the magnetic particle. A surface-active passivating agent may be deposited onto the magnetic particles after attaching the target polymer, but in some cases the passivating agent may be deposited onto the magnetic particles before attaching the target polymer to prevent nonspecific adsorption of the polymer onto the particle surface. In certain implementations, one surface-active passivating agent is utilized, and in certain instances two or more surface-active passivating agents are utilized, to modify magnetic particles. A “passivating agent” also is referred to as a “passivation group” herein.

Alternatively, magnetic particles may be modified to prevent nonspecific adsorption by attaching a passivating agent to the surface using a covalent reaction or affinity tags (FIG. 15B). A passivating agent selected for such implementations sometimes is ionic, zwitterionic, polar, or a large polymeric functional group designed to block or mask nonspecific adsorption interactions between the particle and the PLB, and non-limiting examples include polyethylene glycol (PEG) polymer, poly(2-methyl-2-oxazoline) polymer (PMOXA), hydroxyl, betaine, nitrile, carboxylate, methyl amide, amine, polypeptide, peptide, polysaccharide, polystyrene sulfonate, and polyacrylate. These passivation groups may be attached using covalent coupling chemistry, in which mutually reactive functional groups are attached to the particle surface and passivation group, respectively, and include but are not limited to carboxyl/amine, N-hydroxysuccinimide (NHS) or NHS ester/amine, maleimide/thiol, thiol/thiol, amine/aldehyde, hydroxylamine/aldehyde, and alkyne/azide. Passivation groups may also be attached using affinity tags, such as biotin-streptavidin or antigen-antibody interactions. In an embodiment, PEG with a terminal amine is immobilized onto magnetic particles with carboxylate groups and/or streptavidin on the surface after activating the carboxylate groups with a carbodiimide (for example, 1-ethyl-3-[3-dimethylaminopropyl]carbodiimide (EDC)). Alternatively, PEG with a terminal biotin may be attached to particles with a streptavidin on the surface. PEG polymer of a sufficiently large molecular weight is able to serve as a bulky “polymer cushion” that prevents, via steric effects, the surface of the magnetic particle from approaching near enough to the PLB to allow nonspecific adsorption. A PEG polymer may have a molecular weight of about 0.1 kDa to about 100 kDa, or about 0.5 kDa to about 50 kDa, or about 1 kDa to about 10 kDa, and sometimes the molecular weight of the PEG polymer is about 0.5 kDa, 1 kDa, 2 kDa, 3 kDa, 4, kDa, 5 kDa, 6 kDa, 7 kDa, 8 kDa, 9 kDa, 10 kDa, 20 kDa, or 50 kDa. In certain implementations, one passivating agent is utilized, and in certain instances two or more passivating agents are utilized, to covalently modify magnetic particles or modify magnetic particles via an affinity tag.

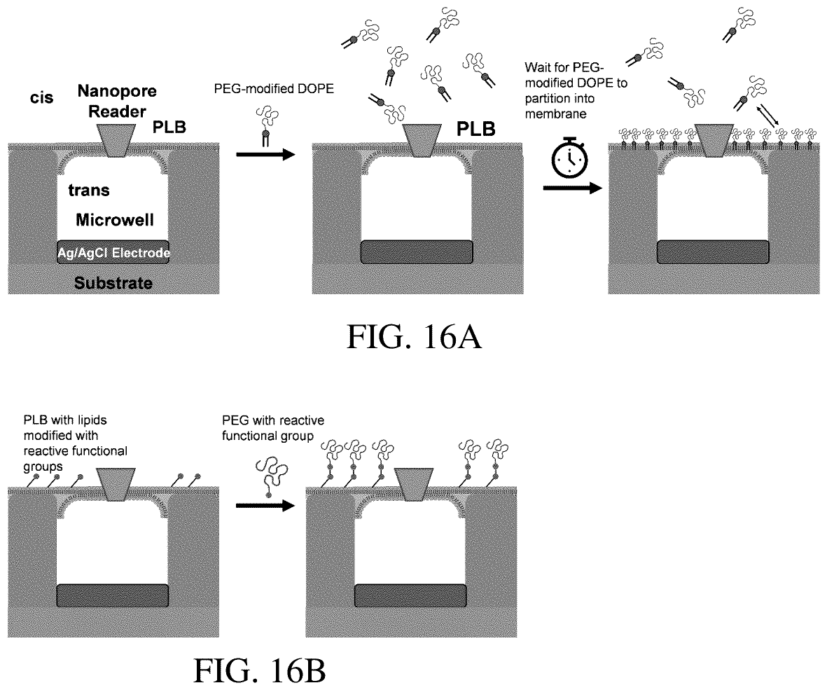

The PLB or membrane may be modified with passivation groups in the “headgroup” region of the PLB to prevent adsorption of magnetic particles. A PLB or membrane may be modified with passivation groups in combination with, or without, modifying magnetic particles with a passivation group. PLBs can be modified by incorporating phospholipids with distinct headgroups selected to reduce attractive interactions between the PLB and the magnetic particles. For instance, lipids with a phosphatidyl serine headgroup may be incorporated into the PLB in order to make its overall electrical charge more anionic in order to repel anionic magnetic particles via electrostatic repulsion. These specific lipid headgroups may include, but are not limited to phosphatidylcholine (PC), phosphatidylethanolamine (PE), phosphatidylglycerol (PG), phosphatidic acid (PA), or phosphatidyl serine (PS). Alternatively, the headgroups of the phospholipids may be chemically modified to attach a functional group designed to reduce nonspecific adsorption of magnetic particles to the membrane, which may include PEG, PMOXA, hydroxyl, betaine, nitrile, carboxylate, methyl amide, amine, polypeptide, peptide, polystyrene sulfonate, polysaccharide, or polyacrylate groups (FIG. 16A). In certain implementations, a surface-active molecule (for example, a surfactant) is incorporated into the PLB in order to modify the surface of the PLB and prevent nonspecific adsorption (FIG. 16B). A surface-active molecule utilized to modify a PLB is typically composed of a hydrophobic component that is able to intercalate into the hydrophobic region of the PLB, and a hydrophilic segment that remains in the solvated headgroup region. Surfactants incorporated into lipid membranes may include small molecule surfactants, including sodium dodecyl sulfate (SDS), Tween-20, Triton X-100, octyl glucoside, digitonin, phospholipids, block copolymers including pluronic, or PMOXA-PDMS, or polymers covalently attached to phospholipids or cholesterol, such as polyethylene glycol modified phospholipids. In certain implementations, a PEG headgroup modified phospholipid (1,2-dioleoyl-glycerophosphoethanolamine) is incorporated into the cis leaflet of the PLB to prevent particle adsorption. The bulky PEG polymer serves to coat the surface of the PLB and prevent adsorption via steric hindrance. The PEG-lipid can be incorporated by first forming the PLB, then inserting a protein nanopore reader into the membrane, then adding PEG-lipid to the cis aqueous volume, and finally waiting a period of time for the PEG-lipid to spontaneously intercalate and coat the PLB. The PEG lipid can be dissolved in an aqueous sample at a concentration of about 0.1 μM to about 1000 μM, or about 1 μM to about 100 μM, and sometimes at a concentration of about 1 μM, 2 μM, 5 μM, 10 μM, 20 μM, 50 μM, or 100 μM. After adding a PEG-lipid to the cis aqueous volume, the cis volume can be mixed and a waiting period of 1 second to 24 hours can be observed to allow the PEG-lipid to spontaneously intercalate into the membrane, with a typical waiting period of about 1 min, 2 min, 5 min, 10 min, 20 min, 30 min, 45 min, or 60 minutes. The molecular weight of the PEG polymer attached to the phospholipid may be about 0.1 kDa to about 100 kDa, or about 0.5 kDa to about 50 kDa, or about 1 kDa to about 10 kDa, and sometimes is about 0.5 kDa, 1 kDa, 2 kDa, 5 kDa, 10 kDa, 20 kDa, or 50 kDa. In certain implementations, one passivating agent is utilized, and in certain instances two or more passivating agents are utilized, to modify a PLB or membrane.

In certain implementations, both the magnetic particles and the membrane are modified in order to prevent adsorption of the particles to the PLBs or membranes. In certain cases the modifications to the particles and the PLBs or membranes are complimentary. For example, coating the particles with 5 kDa PEG and the PLB/membrane with 5 kDa PEG results in a PEG-PEG “cushion” that prevents the intercalation and adsorption of the particles onto and into the PLBs or membrane.

### Chemical Cross-Linking of Membranes to Improve Membrane Stability, Nanopore Reader Stability, and Prevent Nonspecific Adsorption

In alternative embodiments, after inserting a protein nanopore reader into a membrane, and before capturing and sequencing a polymer attached to a magnetic particle, a membrane can be exposed to chemical crosslinking conditions. A membrane sometimes is referred to as a “layer,” sometimes includes one or more types of amphiphilic molecules, sometimes includes one or more types of lipids, and sometimes is a planar lipid bilayer (PLB). “Crosslinking” includes forming covalent bonds linking adjacent amphiphilic molecules in the membrane (for example, phospholipids, block copolymers, fatty acids, or surfactants). “Crosslinking” also refers to polymerizing reactive monomers embedded in the membrane to form extended polymers that act as a scaffold that reinforces the membrane. Crosslinking serves to improve the functionality of the membrane in several ways: 1) Increasing the mechanical stability of the membrane, such that it is more difficult to rupture via vibration or mechanical shock; 2) increasing the voltage stability of the membrane, so that it can be used with a higher applied bias without rupturing; 3) Increasing the lifetime of the membrane, so that ion current measurements can be performed for a longer period of time before the membrane ruptures; 4) Increasing the stability of the nanopore reader within the membrane, so that the nanopore is not disrupted or pulled out of the membrane by the magnetic forces applied to the polymer captured within the reader; and 5) reducing nonspecific adsorption of particles or target polymers by preventing these species from penetrating and intercalating into the membrane. Because a crosslinking process increases membrane stability and makes it more difficult for chemical species to intercalate into the membrane, membranes often are formed and nanopore readers often are inserted into the membrane prior to crosslinking. Crosslinking conditions often are initiated by an external stimulus that can be triggered after membrane preparation and nanopore insertion.

Membranes can be crosslinked using one or more of several techniques. In certain implementations, headgroup region moieties of membranes are crosslinked by applying a polymer from the aqueous volume having individual subunits capable of interacting with multiple headgroup moieties on individual phospholipid molecules through intermolecular interactions or the formation of chemical bonds. Non-limiting examples of such polymers include poly L-Lysine⁴⁷ and actin.⁴⁸ The polymer is able to form a “mesh” over the membrane that increases its rigidity and stability. In certain instances, the membrane is formed using membrane materials (for example, phospholipids, block copolymers, etc.) that have been chemically modified with a polymerizable reactive functional group. This functional group may reside within the hydrophobic region of the membrane, for example, an alkene or alkyne group, that when polymerized form a covalent bond linking multiple lipid molecules together throughout the hydrophobic region after forming the membrane. Non-limiting examples of polymerizable phospholipids include diacetylenic lipids, such as 1-palmitoyl-2,10,12-tricosadiynoyl-sn-glycero-3-phosphoethanolamine (PTPE),⁴⁹ and bis-dienoyl phosphatidylcholine (bis-DenPC) lipids.⁵⁰⁻⁵¹ In certain instances, headgroup regions modified with reactive groups, for example, alkyne, acrylate, methacrylate, or alkene groups, can be polymerized to form extended polymers within the headgroup region. In certain implementations, the membrane can be crosslinked by dissolving hydrophobic reactive monomers in the hydrophobic region of the membrane, and then initiating a chemical reaction to link these monomers together to form an extended polymer “scaffold” within the membrane that serves to improve its stability. Non-limiting examples of monomers that may be used to form a scaffold within the membrane include styrene, divinylbenzene,⁴⁷ butyl methacrylate and ethylene glycol dimethacrylate.⁵² The crosslinking reaction can be performed through radical polymerization initiated by a photochemical redox stimulus. Photochemical initiation may be performed by exposing the membrane to ultraviolet light at a wavelength, often of about 200 nm to about −260 nm, corresponding to the electronic absorption band of the reactive groups or monomers, which produces excited-state species that initiate the chemical reaction. In certain implementations, a chemical reagent known as a “photoinitiator” can be introduced to the membrane, which decomposes when exposed to light corresponding to its absorption band, forming radicals that initiate the polymerization reaction. Non-limiting examples of photoinitiators include benzoyl peroxide, azobisisobutyronitrile (AIBN), 2,2-diethoxy acetophenone (DEAP) and 2,4,6-trimethylbenzoyldiphenyl phosphine oxide (TPO). In certain implementations, a polymerization reaction can be initiated using a redox system with potassium persulfate, sodium metabisulfite, and ferrous sulfate, which serves as a free-radical initiator after being added to the system.⁵³

In certain implementations, membranes are formed using a 1:1 mixture by mass of 1,2-diphytanoyl-sn-glycero-3-phosphocholine (DPhPC) and PTPE diacetylenic lipid at 5 mg/mL in octane. After forming the membranes, protein nanopore readers are inserted into the membrane using a known voltage impulse method. The membrane is then exposed to 254 nm light from a Pen-Ray 0.75″ mercury UV lamp for 5 minutes to crosslink the PTPE lipids within the membrane.

In some instances, molecules and headgroups described herein used to prevent adsorption (for example, nonspecific adsorption) of magnetic particles to the membranes or membranes, can be added before and/or after crosslinking the membranes or membranes.

Addition of a Hard Stop to the Distal End of a Target Polymer to Prevent Escape from the Nanopore after Capture

After capturing a polymer attached to a magnetic particle within a protein nanopore reader, the distal end of the polymer can be associated with a bulky “hard stop agent,” forming a “distal hard stop,” to prevent the polymer from fully escaping the nanopore during a multipassing process. For implementations in which a hard stop agent is an oligonucleotide, the hard stop agent can be referred to as a “hard stop oligonucleotide.” A distal hard stop also is referred to herein as a “hard stop,” and a “hard stop structure.” A hard stop agent can associate with a distal portion or distal end of the polymer to be sequenced (“target polymer”). A distal portion of a target polymer often is disposed in a region about 1 to about 1,000 contiguous nucleotides, or about 1 to about 100 contiguous nucleotides, or about 1 to about 50 contiguous nucleotides, or about 1 to about 20 contiguous nucleotides, from the distal end of the target polymer (for example, a distal region of about 500, 400, 200, 100, 90, 80, 70, 60, 50, 40, 30, 25, 20, 15, 10 or 5 contiguous nucleotides form the polymer terminus).

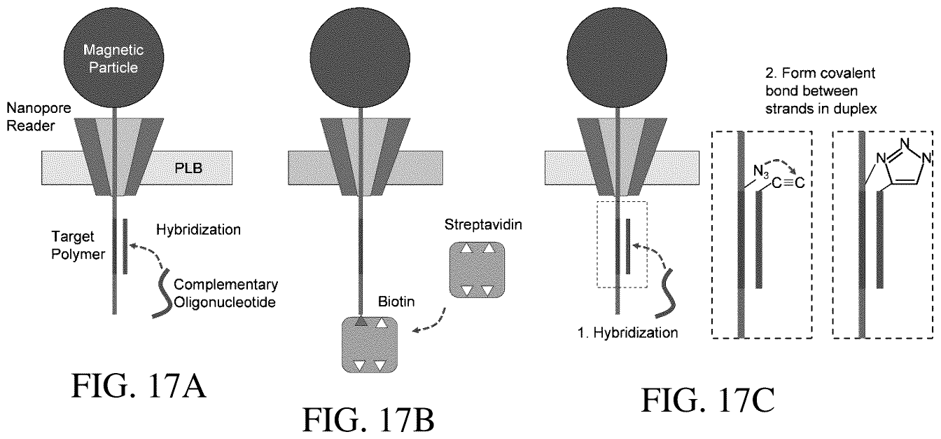

In certain implementations, a polymer attached to a magnetic particle is first captured by the nanopore reader, threaded into the nanopore until it is stopped by the magnetic particle, which is too large to traverse the nanopore reader. After capture by the nanopore, the end of the polymer enters the trans chamber and binds to the hard stop agent, with the hard stop agent forming a distal hard stop structure with the polymer that is too bulky to traverse back out of the nanopore reader in the trans-to-cis direction (FIG. 17). The polymer then can be moved in a cis-to-trans or a trans-to-cis direction up until the distal hard stop or proximal magnetic particle comes in contact with the nanopore reader, using any combination of electrophoretic, electroosmotic, and magnetophoretic forces, without escaping the nanopore. In such implementations, the polymer can be passed back and forth without needing to detect the distal end of the polymer and trigger a change in electrophoretic or electroosmotic force (via the applied voltage), or change the magnetic force, to prevent the polymer from escaping the nanopore reader. A polymer that is threaded into a nanopore reader and prevented from escaping by a hard stop agent on the end of the polymer is sometimes referred to as a “pseudorotaxane” or “rotaxane.” In implementations described herein, a pseudorotaxane or rotaxane contains (i) the proximal magnetic particle on one end of the polymer and in a cis orientation with respect to the nanopore, and (ii) the distal hard stop on the other end of the polymer and in a trans orientation with respect to the nanopore.

A molecule linked to the distal portion or distal end of the target polymer (referred to as a “capture molecule”) can associate with the hard stop agent or a complementary group linked to the hard stop agent. The end of the polymer distal from the magnetic particle can contain an affinity capture or reactive group that is able to bind to or react with a hard stop agent within the trans chamber. A target polymer sometimes includes a polynucleotide (“capture polynucleotide) capable of hybridizing to a complementary polynucleotide of a hard stop oligonucleotide. A capture molecule sometimes includes a linked oligonucleotide (“capture oligonucleotide”) capable of hybridizing to a complementary polynucleotide of a hard stop oligonucleotide. A target polymer sometimes does not include a capture molecule and the hard stop agent associates with the target polymer to form the hard stop structure without a separate capture molecule. A hard stop agent sometimes is associated with a target polymer by a covalent bond and/or non-covalent interaction (for example, hybridization of complementary polynucleotides, interaction between complementary affinity tags).

A hard stop agent used to form a distal hard stop may contain or consist of an affinity tag having a hydrodynamic diameter larger than the diameter of the constriction region of the nanopore reader. Non-limiting examples of a hard stop agent containing or consisting of an affinity tag include streptavidin (FIG. 17A), neutravidin, antibody, antibody fragment, anti digoxigenin, nucleic acid, DNA (FIG. 17B), RNA and an artificial nucleic acid (for example, morpholino, peptide nucleic acid, 2′O-methyl, 2′-fluoro, and locked nucleic acid). A distal hard stop affinity tag molecule often binds to a smaller diameter complementary binding partner installed on the distal end of the polymer that can enter and traverse the nanopore reader, including biotin, an antigen, digoxigenin, or nucleic acid or polynucleotide capable of hybridizing to complementary polynucleotide present in the hard stop agent, for example.

In certain implementations, a hard stop agent used to form a distal hard stop may be installed by reactive functional groups placed on the hard stop and the end of the polymer. Non-limiting examples of reactive functional groups include amine/carboxylate, alkyne/azide, or thiol/maleimide. In certain implementations, a hard stop agent utilized to form a distal hard stop sometimes is a branched polymer, such as a dendrimer, a nanoparticle, a protein, or a large folded DNA structure, such as an i-motif, a G-quadruplex, a DNA duplex, a DNA-RNA duplex, a RNA-RNA duplex, or a DNA origami structure.

In some cases, a combination of both a reactive functional group, and an affinity tag may be used to form a distal hard stop. In certain implementations, the hard stop agent first binds to the target polymer via an affinity tag, which places a reactive functional group in contact with its complementary reactive partner on the target polymer, causing the functional groups to react and form a covalent bond linking the hard stop agent to the polymer (FIG. 17C).

In certain instances, a hard stop agent contains or consists of a nucleic acid oligonucleotide, referred to as a hard stop oligonucleotide. A hard stop oligonucleotide sometimes is about 10 to about 200 nucleotides in length, or about 20, 25, 30, 40, or 50 nucleotides in length. A hard stop oligonucleotide sometimes contains one or more locked nucleotides and sometimes is a locked nucleic acid polymer. A nucleic acid having a polynucleotide complementary to, and capable of hybridizing to, a hard stop oligonucleotide (referred to as a “capture polynucleotide”), often is placed at or near the distal end of the target polymer using a known process (for example, ligation, amplification). The capture polynucleotide sometimes is of the same length as the hard stop oligonucleotide. The capture polynucleotide sometimes is appended to the target polymer at the distal end of the target polymer. The capture polynucleotide sometimes is incorporated within the target polymer upstream of the target polymer distal end (i.e., not appended to the distal end of the polymer), at a distance from the proximal end of the polymer whereby the entirety of the capture polynucleotide resides in the trans chamber after capture and threading through the nanopore reader. The concentration of the hard stop agent in the trans compartment of a sequencing chip or device sometimes is about 100 μM to 1 mM, or about 100 nM to about 50 μM, and sometimes the hard stop agent is at a concentration of about 100 nM, 1 μM, 2 μM, 4 μM, 5 μM, 10 μM, 20 μM, 30 μM, or 50 μM.

In certain implementations, a hard stop agent is streptavidin or neutravidin, located in the trans compartment of the sequencing chip or device, that binds to biotin placed on the distal end of the target polymer sequence, after the target polymer has been captured by and translocated through the nanopore.

A hard stop agent present in a distal hard stop structure can be stripped from the polymer by applying a distal hard stop stripping voltage bias that drives the conjugate in a trans-to-cis direction with sufficient force that the hard stop agent dissociates from the polymer when the hard stop structure contacts the nanopore surface in the chamber. The hard stop agent present in the distal hard stop structure can dissociate from the polymer of the conjugate as a result of the nanopore surface in the chamber forcing the hard stop off the polymer translocating through the nanopore in the trans-to-cis direction. A distal hard stop stripping voltage bias sometimes is greater than about 60 mV, sometimes is about 60 mV to about 1000 mV, sometimes is about 60 mV to about 400 mV, and sometimes is about 60 mV, 70 mV, 80 mV, 100 mV, 120 mV, 140 mV, 160 mV, 180 mV, 200 mV, 220 mV, 250 mV, 300 mV, 400 mV or up to 1000 mV.

Capture and control of translocation by magnetic and electrophoretic forces Magnetic sequencing implementations generally employ electronic control of the magnetic and electrophoretic/electroosmotic force applied to a polymer conjugate for control of the tension applied to the polymer, and control of the direction and velocity of translocation of the polymer through the nanopore reader. These forces can be controlled in real time by a field-programmable gate array (FPGA) or another suitable electronic system, which responds to specific triggering events representing the position and/or velocity of the polymer in the nanopore to alter the forces and thereby controllably translocate the polymer through the nanopore in a manner ideal for sequencing. Methods for controlling the magnetic and electrophoretic/electroosmotic forces and polymer sequencing, are described herein.

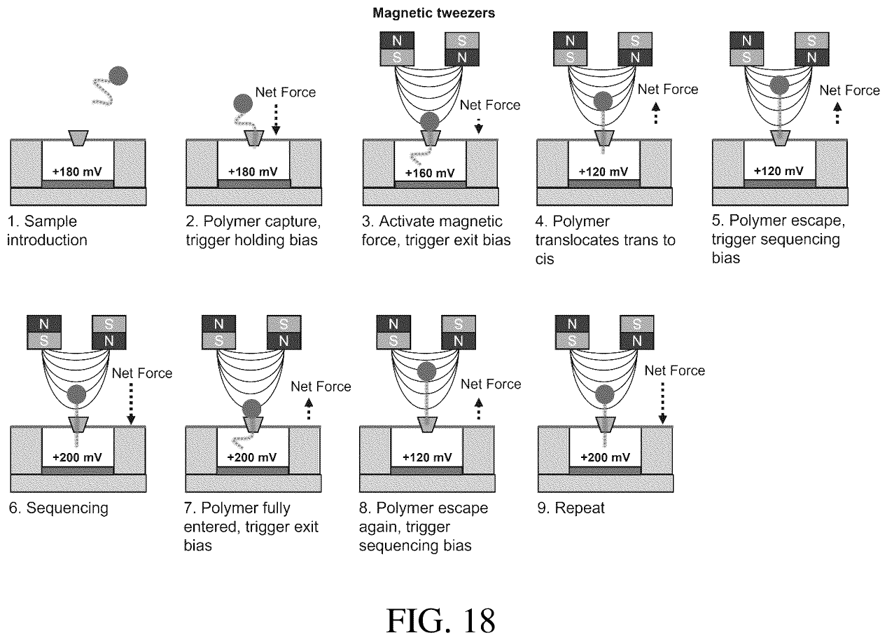

In certain implementations, magnetic tweezers initially are positioned a distance from the sample surface (in the case of permanent magnets), or deenergized (in the case of electromagnets), such that they apply no appreciable magnetic force on the magnetic particle of a polymer conjugate. In certain implementations, the device then initiates a controlled polymer capture and sequencing protocol (for example, FPGA-controlled sequencing as illustrated in FIG. 18):
1. After readers have been inserted, the sample of magnetic particle tagged polymer(s) is introduced to the cis portion of a device (for example, cis buffer volume of the device). A “capture” voltage bias often is then applied between the cis and trans sides of a reader to capture a polymer molecule tethered to a magnetic particle of a conjugate within the nanopore reader. This voltage bias sometime is about 160 mV (mV) to about 240 mV (for example, about 180 millivolts to about 220 mV, or about 190 millivolts to about 210 mV, or about 200 mV, and sometimes is about 80 millivolts to about 1 volt (V) (for example, about 80 mV, 120 mV, 140 mV, 160 mV, 180 mV, 200 mV, 220 mV, or 240 mV, or up to 1 V). This voltage bias drives the polymer into the nanopore reader, where it is captured, where the magnetic nanoparticle on one of its ends is too large to be translocated through the reader.
2. The ionic current in the nanopore reader often is monitored to detect a polymer capture event, which is registered by a decrease in current. After capture, the voltage bias sometimes is reduced to a “holding” bias, which holds the polymer within the nanopore reader and prevents escape from the nanopore reader via the electrophoretic force. The bulky magnetic particle on the proximal end of the polymer serves to prevent the polymer from translocating through the nanopore reader. The holding voltage bias sometimes is about 120 mV to about 200 mV (for example, about 140 mV to about 180 mV, about 150 millivolts to about 170 mV, or about 160 mV), and sometimes is about 10 mV to about 1 V (for example, about 10 mV, 20 mV, 30 mV, 40 mV, 50 mV, 60 mV, 70 mV, 80 mV, 90 mV, 100 mV, 120 mV, 140 mV, 160 mV, 180 mV, 200 mV, 220 mV, 240 mV, 260 mV, or up to 1V). In some instances the holding bias can be the same as the capture bias.
3. A magnetic gradient force or magnetic field is then applied in the opposite direction of the electrophoretic force by lowering the magnetic tweezer closer to the magnetic particle-tagged polymer using the movable magnet mount, in certain implementations. The movable mount can be lowered to a position at a fixed distance (for example, about 100 micrometers to about 10 mm) from the magnetic particle sample to produce the necessary opposing magnetic force (for example, between about 8 pN and about 60 pN). This distance can be determined based on prior experimental calibration of the magnetic force, or theoretical calculations or simulations of the magnetic force distribution. When the magnet is in position, the magnetic force pulling the polymer out of the nanopore typically does not exceed the electrophoretic force pulling the polymer into the nanopore, such that the polymer is still held within the nanopore (for example, the net force on the polymer conjugate, between the electrophoretic force (in the trans direction) and the magnetic force (in the cis direction), is in the trans direction).
4. The applied bias is then switched to an “exit” bias in certain implementations. This exit bias is typically lower than the holding bias (and typically lower than the capture bias) and sometimes is about 80 mV to about 160 mV (for example, about 100 mV to about 140 mV, about 110 millivolts to about 130 mV, or about 120 mV), and sometimes is about 10 mV to about 1 V (for example, about 10 mV, 20 mV, 30 mV, 40 mV, 50 mV, 60 mV, 70 mV, 80 mV, 90 mV, 100 mV, 120 mV, 140 mV, 160 mV, 180 mV, 200 mV, or up to 1 V). When the exit bias is applied, the force imparted on the magnetically tagged polymer conjugate due to the magnetic field generally is larger and overcomes the electrophoretic force applied on the polymer due to the applied exit bias, such that the polymer backs out of the nanopore, in the trans to cis direction (for example, the net force on the polymer conjugate, between the electrophoretic force (in the trans direction) and the magnetic force (in the cis direction), is in the cis direction). During this magnet-induced translocation, the DNA or other polymer typically is pulled taut, in a stretched (for example, fully stretched) conformation due to the magnetic force and opposing electrophoretic force, which can prevent the polymer from coiling or bunching up and can thereby prevent the formation of clogs in the nanopore reader.
5. When the end of the polymer distal to the magnetic particle exits the nanopore, or is near the nanopore in a trans position relative to the nanopore, or in the case of formation of a rotaxane which contains a hard stop on the distal end of the polymer, a controller (for example, FPGA-based controller) in certain implementations recognizes this trigger event (for example, via a polymer exit signature, or a unique current signature of the end of the polymer or adapter tag, or the current signature or current stalling signature of the hard stop) and increases the voltage bias to the “reentry” voltage bias. This reentry voltage bias sometimes is about 160 mV to about 240 mV (for example, about 180 mV to about 220 mV, about 190 millivolts to about 110 mV, or about 200 mV), and sometimes is about 80 mV to about 300 mV (for example, 80 mV, 90 mV, 100 mV, 120 mV, 140 mV, 160 mV, 180 mV, 200 mV, 220 mV, 240 mV, 260 mV, 280 mV or 300 mV), to produce an electrophoretic force that exceeds the magnetic force on the conjugate, causing the polymer to translocate in the cis to trans direction through the nanopore, while typically remaining taut and under tension to prevent coiling, diffusion, or other motion that might interfere with nanopore sequencing (for example, the net force on the polymer conjugate, between the electrophoretic force (in the trans direction) and the magnetic force (in the cis direction), is in the trans direction).
6. During translocation, a sequence-dependent current blocking signature often is measured, providing a single sequencing measurement of the polymer. A current blocking signature (i) can be measured for a polymer translocating in the cis direction after application of a exit voltage bias, or (ii) can be measured for a polymer translocating in the trans direction after application of a reentry voltage bias, or (iii) can be can be measured for a polymer translocating in the cis direction after application of a exit voltage bias and can be measured for the polymer translocating in the trans direction after application of a reentry voltage bias. The polymer conjugate typically translocates through the nanopore in a stretched conformation (i.e., the polymer typically is taut and under tension as it translocates through the nanopore) after application of the entry voltage bias.
7. When the proximal end of the polymer tethered to the magnetic particle reaches the nanopore, the controller (for example, a FPGA-based controller) often recognizes this trigger event via a stalled ionic current (because the bulky magnetic particle cannot enter the nanopore), or a current signature unique to the sequence of the end of the polymer or adapter. Upon recognition of the stalled current, the FPGA controller can reduce the voltage bias to the “exit” voltage bias, where the magnetic force again overcomes the electrophoretic force, causing the polymer to translocate back out of the nanopore in the trans to cis direction in a stretched conformation.
8. When the distal end of the polymer again exits the nanopore, or is near the nanopore in a trans position relative to the nanopore, or in the case of formation of a rotaxane of the target polymer, a hard stop is detected, the controller (for example, FPGA-based controller) steps the voltage bias back to the reentry voltage bias to translocate the polymer back through the nanopore reader in the cis to trans direction, which allows for multipassed sequencing of the polymer.
9. This process can be repeated to sequence the polymer a single time, or multiple times (between 2 and 10,000 multipasses) in order to achieve a desired sequencing accuracy.

The current signature of the reader can be used to sequence the polymer only as it is translocating through the nanopore reader from the outside to the inside of the well (in the cis to trans direction). The current signature of the reader can be used to sequence the polymer as it is translocating back through the reader from the inside to the outside of the well (in the trans to cis direction) as it is pulled back through the reader by the applied magnetic force. The current signature of the reader can be used to sequence a polymer as the polymer is translocating in both directions, or any combination thereof. Repeated polymer sequence determinations from these translocation directions in any combination can be used to produce a consensus sequence with higher accuracy than a single sequence determination would allow.

In the case of polymers that form a duplex structure, such as DNA-DNA, DNA-RNA, or RNA-RNA duplexes, or polymers with double-stranded capture and/or anchoring tags (proteins with ligated adapters), the complementary polymer strand can be removed to form a single-stranded polymer within the nanopore reader, before sequencing the captured single-stranded polymer. In certain implementations, a single stranded region of the target strand in the duplex first can be captured by the reader by applying a capture bias. This single stranded region can be a free single-stranded end of the polymer, or a single-stranded capture tail added to the target polymer via a ligated adapter. After capturing the single stranded end of the polymer, the bias voltage can be maintained to induce duplex dissociation with the applied electrophoretic force. Alternatively, the bias can be increased to a stripping voltage of about 200 mV, or about 100 mV to about 1 V (for example, about 120 mV, 140 mV, 180 mV, 200 mV, 220 mV, 240 mV or up to 1V) to increase the electrophoretic force and induce duplex dissociation and translocation of the target polymer into and through the nanopore reader, where it is captured. In certain implementations, when a controller (for example, FPGA-based controller) detects that the polymer has fully entered the well and completely stripped off the complementary polymer strand, via a reader ionic current trigger event, such as stalled current, or a unique sequence signature of the polymer or ligated adapter, the controller executes the sequencing protocol by controlling the voltage bias, magnetic force, or a combination thereof to pass the polymer back through the nanopore reader to sequence it a single time, or multiple times (during about 2 to about 10,000 multipassing events). Each event of a multipassing process typically is translocation of a polymer through a nanopore in one direction, where the polymer can be sequenced during some or all of the events (for example, a polymer can be sequenced in one direction only, in both directions or a combination thereof).

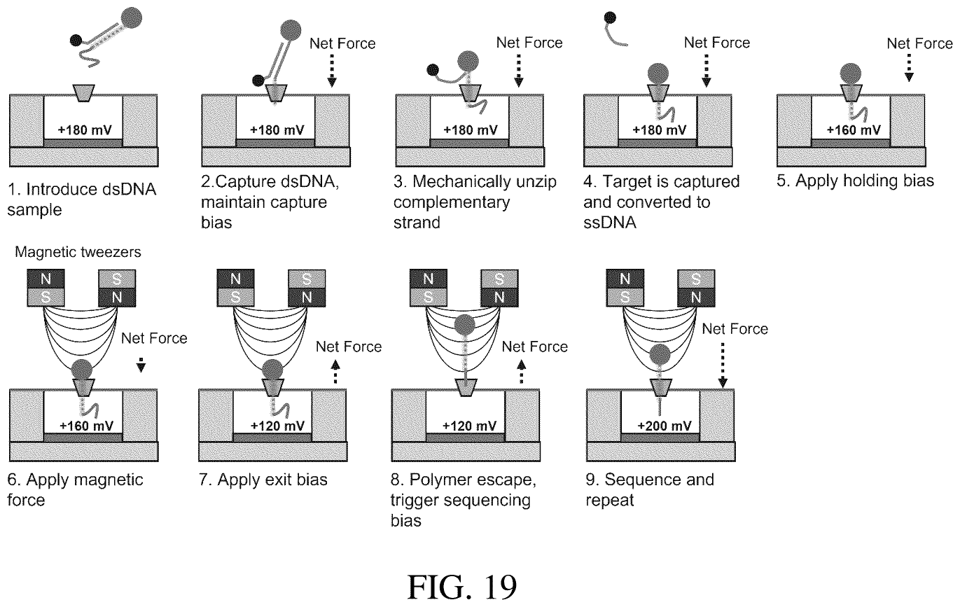

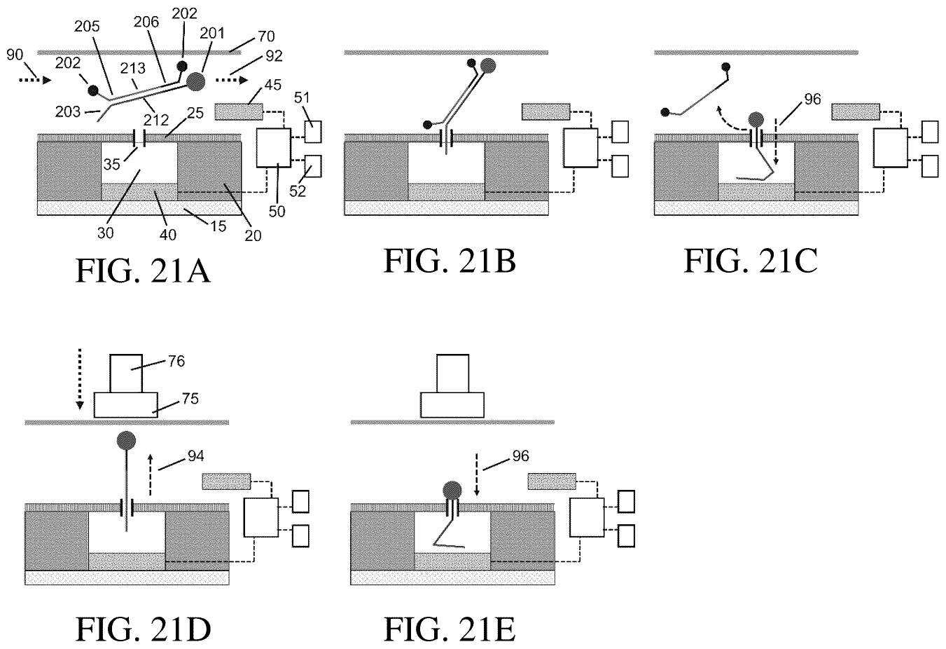

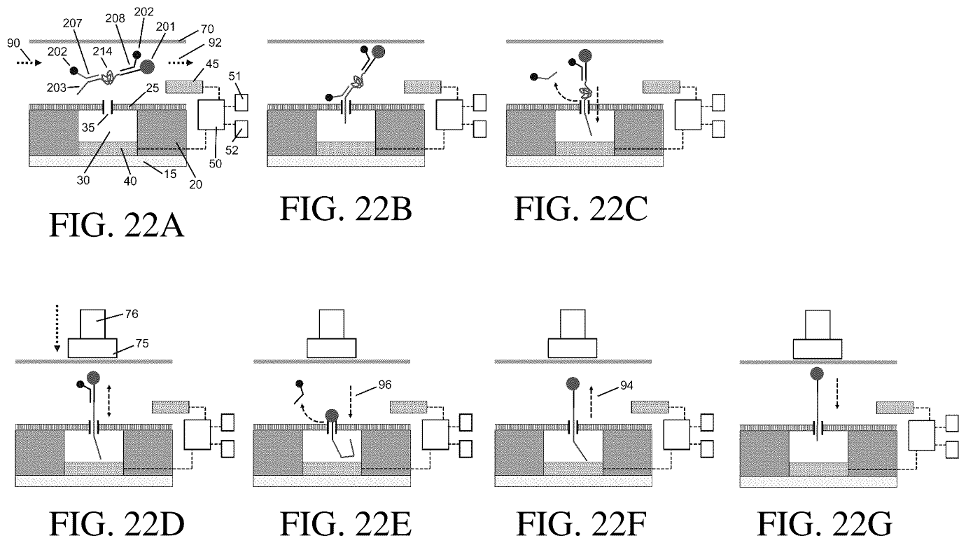

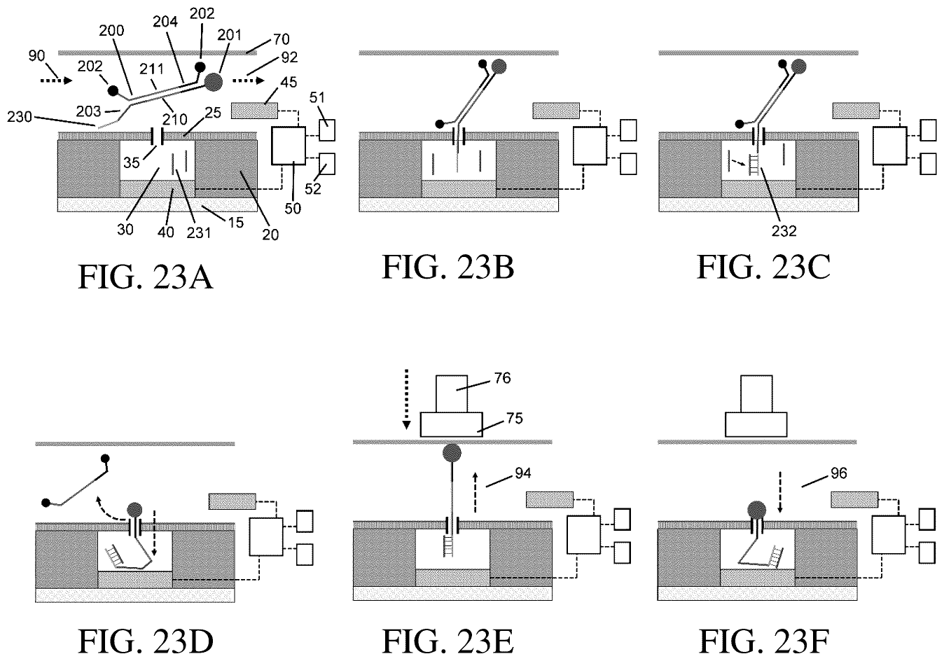

Non-limiting examples of multipass magnetic sequencing processes are illustrated in FIGS. 19-23, a non-limiting implementation is described hereafter:
1. Introduce prepared DNA sample (for example, a prepared DNA sample library), in which the target DNA is attached to a magnetic particle, into the test cell or chamber.
2. The control module initiates the capture protocol by selecting the capture bias level from the bias module and applying it across the nanopore reader.
3. The nanopore current as measured by the amplifier is monitored in quasi-real time by the control module for a capture signature.
4. The capture or overhang tail on the DNA is captured by the nanopore and the complementary strand is mechanically removed by the applied capture bias or a higher stripping bias such that the target DNA strand translocates through the nanopore, until the magnetic particle on the end of the target DNA strand reaches the nanopore.
5. Once the control module detects a DNA molecule capture signature it instructs the bias module to immediately switch to a holding bias to hold the DNA molecule within the nanopore reader.
6. The control module then activates a magnetic field that is applied to the apparatus/nanopore reader/captured magnetic particle.
7. The control module instructs the bias module to change the bias to the exit bias and the DNA strand controllably backs out of the nanopore reader, trans to cis.
8. Once the DNA strand reaches its end, exiting or almost exiting the nanopore reader, the control module instructs the bias module to apply a reentry bias to controllably drive the DNA strand back into the nanopore reader, cis to trans.
9. The DNA can be sequenced in one translocating direction (i.e., trans-to-cis direction or cis-to-trans direction) or both translocating directions (i.e., trans-to-cis direction and cis-to-trans direction). Although the amplifier and DAQ system are measuring and recording the nanopore current continuously the most relevant data of the sequence is obtained in steps 7 and 8.

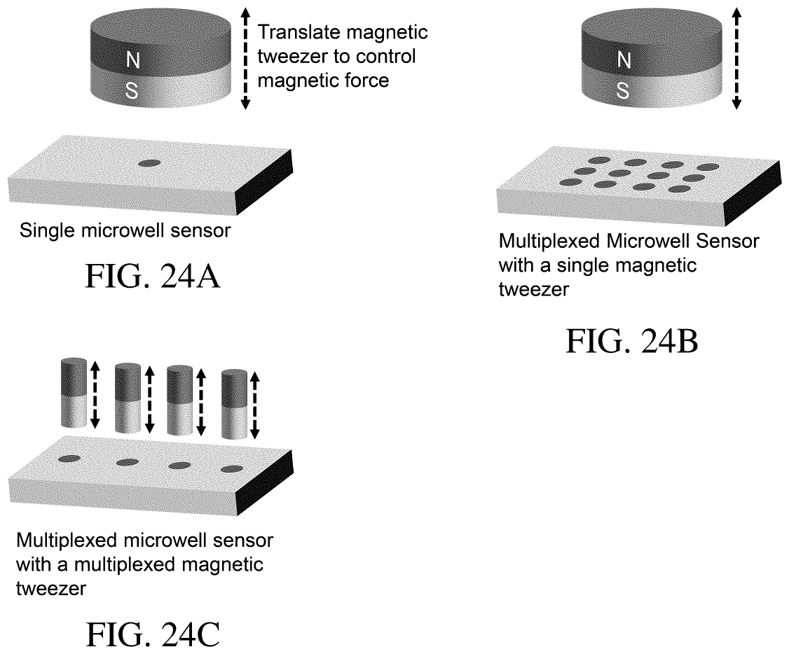

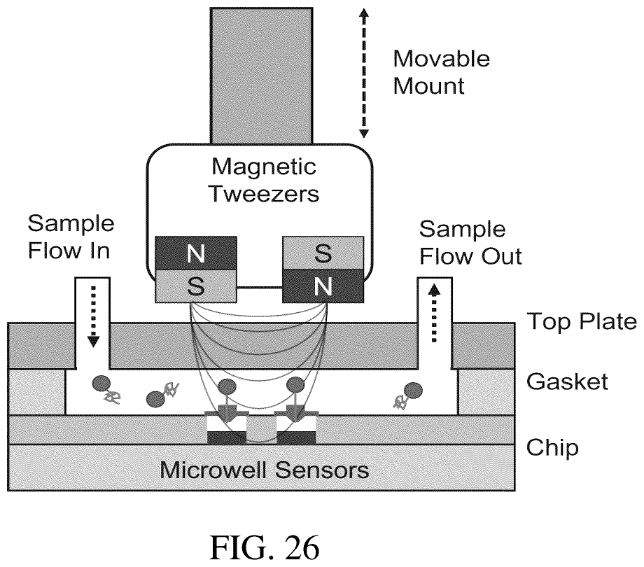

Multipass magnetic sequencing can be performed on a chip that has a single well sensor, or multiple well sensors (for example, illustrated in FIGS. 24-26). A device with multiple wells can have about 2 to about 100,000 individually addressable wells, can have about 100 to about 10,000 individually addressable wells, and sometimes has about 2, 4, 8, 16, 32, 64, 128, 256, 512, or 1024 individually addressable wells. On devices with more than one microwell sensor, a controller (for example, FPGA-based controller) generally is capable of adjusting the voltage bias to each microwell sensor individually. In an implementation of a multiplexed multipass magnetic sequencing device, a single magnetic tweezer device can be used to simultaneously apply force to many magnetic particle-tagged polymers individually captured by nanopore readers in multiple microwell sensors. Devices with a single magnetic tweezer device shared between multiple microwell sensors can apply a fixed magnetic force shared amongst all microwells, and a net polymer translocation force can be varied by varying the electrophoretic force separately at each sensor. In certain implementations, each microwell sensor is associated with its own dedicated magnetic tweezer device, and each of tweezer devices sometimes is individually controlled to control the magnetic force at each microwell sensor. Multiplexed devices with separate magnetic tweezers for each sensor can make use of electromagnet coils near each microwell sensor, or small permanent magnets each with individual motion control to vary the distance between the magnet surface and the magnetic particle tagged polymer. On devices with individually controlled magnetic tweezers for each microwell sensor, the net force on the polymer can be controlled by varying the magnetic force (either by the electromagnet current or the magnetic tweezer distance), the electrophoretic force, or both forces simultaneously.

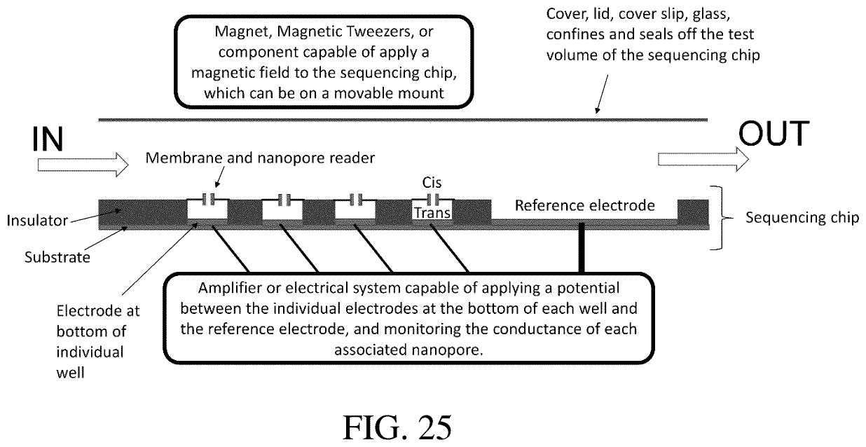

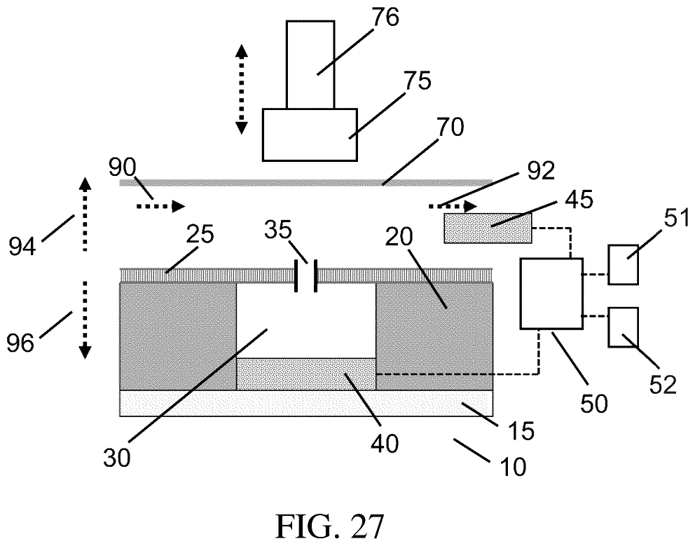

Overviews of non-limiting examples of magnetic sequencing device are illustrated in FIGS. 25-27. The sequencing chip includes multiple channels, where each channel has an individual well with an electrode at the bottom. The chip can utilize an integrated or external reference electrode. Individual channel electrodes often are electrically connected to amplifiers to measure the ionic current through the channels. Each channel can be biased independently using a low-noise, high-speed DC bias circuit controlled by an FPGA. For low channel count systems, less than 16 channels, a COTS integrated circuit or a discrete based amplifier can be used. For higher channel count systems, a multichannel ASIC can be used. A multichannel ASIC with the electrodes and wells integrated into the top layer of the ASIC can be utilized. A device can include a flow cell comprising the cis portion of the device, which includes an inlet and outlet port to allow samples to be flowed into the device for testing. This flow chamber can include a top plate composed of a non-ferrimagnetic material that serves to confine the sample to the device and prevent magnetic particles from contacting the magnetic tweezers.

In a multipass implementation, a DC bias often is used to electrophoretically move the DNA molecule until it is captured by the nanopore reader and a capture signal is detected, after which the bias is changed to a holding state that holds the captured DNA molecule within the nanopore. Then a magnetic field often is added and the bias is altered such that the DNA molecule translocates back through the pore until it escapes. Once the exit signal is detected the process can be restarted quickly to re-measure the same DNA molecule.

In multipass implementations, DNA is moving at relatively high velocity through the nanopore reader. To make measurements of ionic current through the pore the amplifier and data acquisition system typically have a wide bandwidth. Data often is monitored in quasi-real time for detection of capture and exit signatures, for example, and the DC bias typically is changed rapidly in response to a trigger event. Rapid DC bias changes can be accomplished by incorporating on-board decision-making hardware such as an FPGA, microprocessor, or ASIC. An FPGA can be utilized in certain implementations as it is readily reconfigurable in software, and an FPGA can be replaced by an ASIC-based solution in certain implementations. Once a trigger event is detected, the FPGA or ASIC can change the DC bias with a specialized high-speed DC bias circuit.

A specialized high-speed DC bias circuit incorporated in a system typically satisfies the requirements of low-noise and high-speed. These two requirements are not necessarily compatible with each other because to lower the noise the bias often is low-pass filtered with a low corner frequency, which can introduce time delays. To solve this problem a multiplexer with several, highly filtered, preset DC bias voltages can be utilized. In such implementations, by the time a FPGA needs to select a specific bias voltage, the specific voltage bias has already settled and is well filtered. DC bias can be set by digital-to-analog converters (DAC) but can be replaced with fixed hardware solutions. For multipass implementations, typically a minimum of four preset voltage biases are utilized (for example, capture bias, hold bias, exit bias, reentry bias), each of which is applied after detection of a trigger event (for example, capture event, exit event, reentry event).

In certain implementations, a control module is configured to detect a trigger event and transmit a preset voltage bias level after detection of the trigger event. In certain instances, detecting a trigger event includes detecting a current measurement, and sometimes the current measurement is a high bandwidth current measurement. A high bandwidth current measurement by the system sometimes is a current measurement at about 100 kiloHertz (kHz) to about 250 kHz (for example, for a single channel), about 25 kHz to about 75 kHz (for example, for about 2 to about 100 channels) or about 1 kHz to about 10 kHz (for example, for over 100 channels). In certain implementations, one or more amplifiers in the system each have a bandwidth of about 10 Hz to about 1 kHz and the system implements one or more software routines that permit a high bandwidth current measurement. After a trigger event is detected, in certain implementations, a high-speed DC bias circuit changes the DC bias applied at a sensor site (for example, well) in a time frame of about 0.01 milliseconds (ms) to about 5 ms, about 0.05 ms to about 1 ms, about 0.075 ms to about 0.25 ms, or about 0.1 ms. A bias module sometimes produces a highly filtered preset voltage bias level having a noise of about 20 nV/rtHz or less after filtering.

### Polymer Sequencing

In certain implementations, a system/device is set up with membranes over and/or spanning all or a portion of wells on a sequencing chip and individual nanopore readers within all or a portion of those membranes. A chip typically includes a cover between the wells and the magnet that defines a cell, in which solution containing magnetic particle-polymer conjugates is added. The magnetic particle-polymer conjugates sometimes are part of a sample or library for sequencing. Such a solution can be filled once for sequencing in a test cell or can be flowed before, during and/or after sequencing in a flow cell. The test cell or flow cell can be filled with a test cell solution or flow cell solution before, during or after sample is added.

In some implementations, before forming membranes over wells and inserting individual nanopore readers into membranes, the trans well volumes may be filled with molecules used to bind to the distal end of the polymer and form hard stops. The molecules that form hard stops can be added to the trans chamber at a concentration of about 100 μM to about 1 mM, or about 100 nM to about 50 micromolar, and sometimes a hard stop agent is at a concentration of about 100 nM, 1 μM, 2 μM, 4 μM, 5 μM, 10 μM, 20 μM, 30 μM, or 50 μM.

In some implementations, after forming membranes spanning all or a portion of wells on a sequencing chip, a solution of surfactant molecules, such as PEG-modified lipids, may be added to the cis chamber of a chip or device. This solution may be retained in the chamber for a period of time of about 1 second to about 24 hours, or about 1 minute to 60 minutes, or about 1 minute to about 30 minutes, to allow the surfactant molecules to partition into the membrane and passivate the membrane surface, which can reduce nonspecific adsorption of particles onto the membrane. The surfactant solution may be rinsed out of the sample chamber, and/or can be maintained in the samples containing polymer to be sequenced to maintain a constant concentration of surfactant in contact with the membrane. Surfactant is often added to the chamber at a concentration of about 0.1 μM to about 1000 μM, or about 1 micromolar to about 100 micromolar, and sometimes at a concentration of about 1 μM, 2 μM, 5 μM, 10 μM, 20 μM, 50 μM, or 100 μM.

The library or sample to be tested, containing polymers already attached to the magnetic particles, typically is added to the test cell or flow cell on cis side of the sequencing chip, often through an injection port, inlet, or front side of the test or flow cell, directly (for example, via a pipettor) or via a pump. The final concentration of the polymer within the test or flow cell, which is ready to be sequenced, often is about 1 zeptomoles to about 1 micromoles of polymer conjugate, and sometimes is about 1 femtomole to about 10 picomoles. Prior to loading in the test cell or flow cell, polymers typically are immobilized onto magnetic particles such that the number of polymers per particle sometimes is about 1 polymer per particle to about 1×10⁶ polymers per particle, and sometimes is about 10 to about 104 polymers per particle. At these ratios, the amount of magnetic particles containing polymer is about 0.1 yoctomoles to about 100 nanomoles of magnetic particles with attached polymer, and sometimes is about 0.1 attomoles to about 90 femtomoles of magnetic particles.

A magnet or magnetic system of the system/device typically is in a first state, in which there is a negligibly low magnetic field and low to no resulting magnetic force near the chip, when sample is applied to the system/device. In the first state, a magnet sometimes is positioned away from the sequencing chip at a position in which there is a negligibly low magnetic field and low to no resulting magnetic force near the chip.

After the sample to be sequenced is introduced into the test cell or flow cell, an initial capture voltage bias is applied to capture individual polymer molecules into individual nanopore readers in the array. After a polymer is captured, a holding bias is set for each specific nanopore reader that has captured a polymer, to hold the polymer within the reader and prevent its escape back into solution. The holding bias can be the same as the capture bias, in some instances. The capture process often proceeds until at least a fraction, or all, of the active and intact nanopore readers have captured a polymer. This fraction often is about 0.1% to 99.9% of the active nanopore readers, and sometimes is about 30% to about 90% of the active nanopore readers, and sometimes a specified fraction is attained after which the process further proceeds. In certain implementations, after capturing a polymer in a nanopore, the polymer may be held in the nanopore using the holding bias for set period of time to allow the formation of a rotaxane, forming a hard stop on the distal end of the polymer. This time delay is often about 0.1 second to about 1 hour, and is sometimes about 30 seconds to about 5 minutes.

After at least a fraction of nanopore readers have captured a polymer and are being held at the holding bias (or capture bias), the magnet or magnetic system is then altered to a second state in which it applies a magnetic force to the captured polymers/particles. In a second state, a magnet sometimes is mobilized to a second position, or otherwise activated, and sometimes is positioned closer to the sequencing chip than in the first state or first position to apply a magnetic force to the captured polymers/particles. A control module of the system sometimes transmits a magnet activation signal (for example, to a DAC) to convert the magnet from the first state to the second state. The captured polymers then can be controllably sequenced, as described herein. Associated captured polymers can be sequenced by a single pass or single read, into or out of the nanopore, or can be sequenced by multiple passes or rereads (i.e., multipassing).

After the sequencing of a given captured polymer is complete in a first sequencing run, the sequenced polymer conjugates can be released back into the test cell or flow cell solution. For implementations in which a distal hard stop is incorporated on the captured polymer, a combination of electrophoretic/electroosmotic and magnetic forces may be applied to the polymer in the trans-to-cis direction to induce dissociation of the distal hard stop from the polymer and allow the polymer to exit the nanopore and be released back into the test cell solution. After the first sequencing run is complete, the magnet or magnetic system often is placed back into the first state (for example, away from the sequencing chip) and a second sequencing run can commence, where a capture bias is applied to the nanopore readers (for example, all of the nanopore readers) on the sequencing chip and polymers are captured. In certain implementations, polymers from the original sample that were already sequenced or fraction thereof, and/or polymers that were not initially captured in the first round of sequencing or fraction thereof, are captured and held within the nanopore readers. In certain instances, a delay period is implemented to allow polymers tethered to magnetic particles to diffuse and mix in the sample chamber before capturing polymers again to avoid capture or reduce capture frequency of the same polymer molecules that were sequenced previously. After capture, the magnet or magnetic system is placed in the second state (for example, close to the sequencing chip surface). At this point the captured polymers can be sequenced before placing the magnet back into the first state, thereby completing a second sequencing run. After the second sequencing run is completed, the system can be reset, which often includes placing the magnet into the first state for capture of another round of polymers, after which a third sequencing run can commence if desired. Any suitable number of sequencing runs can be implemented for a particular sample (for example, 1, 2, 3, 4, 5, 6, 7, 8, 9, 10, 20, 30, 40, 50, 60, 70, 80, 90 or 100 sequencing runs). After a given sample is fully sequenced and the magnet or magnet system is set back to the first state (for example, away from the sequencing chip), the test cell or flow cell or cis side of the sequencing chip can be flushed clear of that sample, before a new sample or new library is added to the system for sequencing. This process can be iteratively repeated.

Certain implementations of magnetic multipass sequencing include:
1. The magnetic particle-tagged polymer translocation direction and velocity can be controlled by varying the magnitude of the magnetic force while maintaining a fixed electrophoretic force (for example, by holding a constant voltage bias). The magnitude of the magnetic force can be controlled by varying the distance between the magnetic tweezers and the magnetic particle tagged polymer using a motorized mount controlled by a FPGA. A FPGA can automatically or semi-automatically control movement of the magnetic tweezer using current measurement trigger events, which can include polymer exit signatures, and/or current measurements/signatures associated with the polymer escaping into bulk solution, the polymer stalling, and the like. FPGA control of the magnetic force also can be based on other measurements of the reader ionic current response, including the translocation velocity.
2. The magnetic particle-tagged polymer translocation direction and velocity also can be controlled by varying the magnitude of the magnetic force while maintaining a fixed electrophoretic force (for example, by holding a constant voltage bias). The magnitude of the magnetic force can be controlled by varying the current to an electromagnet coil used to apply the external magnetic field, in certain implementations. The current in the electromagnet can be controlled by a FPGA, automatically or semi-automatically, based on reader ionic current trigger events, which can include polymer exit signatures and/or current measurements/signatures associated with the polymer escaping into bulk solution, the polymer stalling, and the like. FPGA control of the magnetic force also can be based on other measurements of the reader ionic current response, including the translocation velocity.
3. The magnetic particle-tagged polymer translocation direction and velocity also can be controlled by varying both the magnetic force and the electrophoretic force in combination, by the FPGA, with automatic or semi-automatic control based on reader ionic current trigger events and/or metrics from the current response, such as translocation velocity.

In certain implementations, after a polymer of a conjugate is captured and optionally held in a nanopore reader, the polymer translocates through the nanopore in the trans-to-cis direction (i.e., the cis direction) and/or in the cis-to-trans direction (i.e., the trans direction) (i) under a constant magnetic force, (ii) under a constant magnetic field, (iii) without modifying a magnetic field exerted by a magnet, (iv) without altering a position of a magnet, (v) without altering a position of a magnet to more than two positions, (vi) with the conjugate unattached to, or unassociated with, a solid support, (vii) with the conjugate unattached to, or unassociated with, a moveable solid support (for example, unattached to or unassociated with a translatable stage or plate), or (viii) a combination of two, three, four, five, six or all of (i), (ii), (iii), (iv), (v), (vi) and (vii). In certain instances, a system does not include a moveable solid support, often does not include a translatable stage or plate, and often does not include a translatable stage or plate capable of being disposed in three or more positions. In certain instances, a system does not include a magnet disposed on a support capable of being disposed in three or more positions.

In certain implementations, after a polymer of a conjugate is captured and optionally held in a nanopore reader, the polymer translocates through the nanopore in the cis direction and/or the trans direction under a motivation force, where the motivation force consists of the net force between (i) the electrophoretic and/or electroosmotic force and (ii) the magnetic force, on the conjugate. In certain implementations, a net force between (i) the electrophoretic and/or electroosmotic force and (ii) the magnetic force, on the conjugate, is modulated by modulating an electric field at a nanopore in a system. In certain implementations, a net force between (i) the electrophoretic and/or electroosmotic force and (ii) the magnetic force, on the conjugate, is modulated by modulating an electric force on the conjugate at a nanopore in a system. In certain implementations, a net force between (i) the electrophoretic and/or electroosmotic force and (ii) the magnetic force, on the conjugate, is not modulated by modulating a magnetic field at a nanopore in a system. In certain implementations, a net force between (i) the electrophoretic and/or electroosmotic force and (ii) the magnetic force, on the conjugate, is not modulated by modulating a magnetic force on the conjugate at a nanopore in a system.

### Triggers for Voltage Changes

In certain implementations, after a polymer is captured by a nanopore reader and is held in place as the magnetic field is put into position and/or correctly oriented, the voltage bias is then decreased to the “exit sequencing bias” such that the captured polymer starts to translocate back out of the nanopore, in the trans-to-cis direction, while under tension, at a sequence resolvable rate. Once almost the entirety of the polymer has been pulled out of the nanopore reader almost to or actually to its very end, the system can assess for a pre-defined trigger that when recognized can trigger the applied bias to immediately be switched to a “recapture sequencing bias”, i.e. a bias that can exert an electrophoretic and/or electroosmotic force onto the polymer which overcomes the magnetic force exerted on the polymer such that the polymer is pulled back into the nanopore reader in the cis- to trans-direction under tension at a sequence resolvable rate. A recapture voltage bias also is referred to herein as a reentry voltage bias. The polymer is then pulled all the way back into and through the nanopore until the magnetic particle on its end reaches the nanopore reader at which point this process can be performed repeatedly.

Pre-defined triggers that can be utilized to trigger the system to automatically switch from the exit sequencing bias to the re-capture sequencing bias include but are not limited to a specific current level and/or noise level as well as an associated time component with either or both, a specific current and/or noise level pattern, event current levels and/or durations, and/or the escape of the end of the polymer through the sensing zone of the nanopore reader but still within the vicinity of the nanopore reader such that it can be recaptured. For implementations in which a distal hard stop is incorporated on the polymer, a pre-defined trigger sometimes is a stalling current signature indicating that the distal hard stop has reached the trans side of the nanopore and halted translocation, or a unique current signature or noise level associated with the hard stop reaching the trans end side of the nanopore and has halted translocation. Additional triggers include but are not limited to filtering the data to assess small changes in resistance as the strand vacates the space immediately outside the pore's trans opening, monitoring the rate of change of the associated current response and triggering from a specific rate of change, recognition of a current signature from a chemical label added near the end of the strand, and machine learning designed to learn the end pattern in real-time as the strand is sequenced.

Similar pre-defined triggers can be used to assess when the polymer has fully translocated through the nanopore reader, reaching its end, with the polymer no longer able to translocate because the magnetic particle has reached the cis reader entrance. Here, the current level and/or associated noise can stop changing and settle on a steady-state response, and this steady current level can be used to indicate that the magnetic particle has stopped at the cis entrance. However, one or more or all triggers stated above also can be used, including identifying chemical tags ligated to the polymer on the end proximal to the magnetic particle, a unique current signature of the anchoring tag, in addition to waiting for pre-defined set periods of time, associated with drawing all potential polymers into the nanopore reader until they reached their associated magnetic particle.

One or more of the following non-limiting events, alone or in combination, can be utilized as a trigger event in a magnetic sequencing system: (i) set period of time; (ii) specific current and/or noise level; (iii) specific current and/or noise level for a specific period of time; (iv) exit signature of the end of the polymer coming out of one of the readers, before fully escaping out into bulk solution; (v) specific sequence of the polymer; (vi) stalled current indicating the magnetic particle tag is in contact with the reader; (vii) stalled current indicating a distal hard stop on the distal end of the polymer is in contact with the reader; (viii) signature of a molecular marker added to the polymer at a specific or arbitrary location (for example, in the form of an adduct); (ix) translocation velocity of the polymer; or (x) a combination of two, three, four, five, six or all of events (i), (ii), (iii), (iv), (v), (vi), (vii), (viii), and (ix).

After a trigger event is detected by a magnetic sequencing system, a control module of the system can automatically or semi-automatically switch or alter an applied voltage bias and/or magnitude of the magnetic force (via magnetic tweezer position or electromagnet current), and thereby change the net force on the polymer conjugate, and thereby change the translocation velocity of the polymer and/or drive the polymer in the opposite direction to induce multipassing or flossing of the polymer back through the reader.

### Electronics

An electronic system in certain implementations can include independent resistive feedback amplifiers (independent capacitive feedback amplifiers also can be utilized) that each independently measure the ionic current through the nanopore readers, inserted into their respective membranes, over each individual well on the sequencing chip, with the integrated electrodes typically at the bottom of each well. Electrical connections often are made between the chip and the amplifier through spring-loaded connectors (pogo pins). A chip can be wire bonded to a printed circuit board (PCB) and header/socket type connections can be utilized. Delicate wire bonds often are covered with an epoxy/encapsulant so they are not knocked loose. A chip can be wire bonded into an appropriate integrated circuit (IC) package and a test socket can be used to connect to the IC and electronics. The sockets often include contacts having spring wire and often are soldered to a PCB. The input amplifiers often are designed for low-noise, high-bandwidth measurements of the ionic current through the nanopores, in a current-to-voltage converter circuit.

A host computer with application software and a data acquisition (DAQ) device often controls the magnetic force sequencing system. The DAQ hardware typically includes ADCs (analog to digital converters) for acquiring analog signals, DACs (digital to analog converters) for providing bias, and an onboard FPGA for quick decision making. Data is archived to the host storage for post processing, analysis, and visualization.

Voltage bias often is applied to the sequencing chip electrodes through the amplifier input and a shared reference electrode in a bath or test solution (cis solution). In certain implementations, the reference is held at zero volts or connected to analog ground, but it does not have to be. The voltage bias typically is applied by a low-noise source that can rapidly change the voltage bias. A specialized circuit utilizing a multiplexer and fixed DC levels typically is utilized. DACs on the DAQ can be used to provide preset bias levels. These levels then can be filtered and attenuated to produce low-noise sources. A multiplexer can then select any one of these manually or through an automated routine controlled by a FPGA. One multiplexer circuit generally is paired to each amplifier, which configuration allows each nanopore to be biased independently through the associated amplifier.

The output signals from the input amplifiers generally are amplified, filtered, and digitized by the DACs. A FPGA often is employed to process the digital data and automate the system by detecting events/triggers such as DNA capture, release, end of strand, and the like, as described herein. When these events are detected, a FPGA often initiates a signal to change the bias multiplexer selection to the next appropriate bias level. In this manner a FPGA can be used to thread/translocate the strand through the nanopore and then back out, threading it back and forth electrophoretically and or electroosmotically, against the applied magnetic force, so the strand can be read/sequenced multiple times (“multipassed”).

Configurations described above often are applied to smaller arrays of multiplexed sensors (for example, on a 4, 8, 16, 25, 96, 100 channel system) using printed circuit board (PCB) amplifiers. In a larger scale system (for example, greater than 16 channels, or hundreds or thousands of channels) an ASIC often is utilized that incorporates the above-described functionalities for multiple channels. A capacitive feedback amplifier often is utilized as large resistors are not typically incorporated into an ASIC. The ADC, DAC, and biasing often are standard elements in an ASIC. A FPGA typically is not compatible with an ASIC, and an FPGA can be replaced with a fixed hardware counterpart in an ASIC, in which the FPGA functionality is emulated with digital gates and devices. An external FPGA also can be utilized with an ASIC. In some instances a sequencing chip can be built on top of an ASIC, forming an integrated ASIC, a system-on-a-chip, or a lab-on-a-chip, where the top layer of the ASIC is reserved for the wet chemistry, or the electrodes, wells, membranes and nanopore readers.

### Translocation Velocity of a Polymer Under Tension Through a Nanopore

Polymer translocation velocity can be represented as a transit time per monomer unit, in units of time per monomer, or as a rate, in units of monomers per time. A monomeric unit typically is a nucleotide or nucleoside for RNA or DNA, or is an amino acid for a peptide or protein. Because these monomeric units can be different in length for different types of polymers (for example, DNA, RNA, or proteins), a velocity in monomeric units per time can represent different linear velocities represented in distance per time, i.e., nanometers (nm)/millisecond (ms). A velocity typically represents the magnitude of the polymer velocity translocating through a nanopore reader in the cis-to-trans or trans-to-cis direction. In the case of multipassing, where a polymer is passed through a nanopore reader multiple times in each direction, the polymer can translocate through the nanopore reader at the same or different translocation velocity in the cis-to-trans and trans-to-cis directions. A transit time sometimes is an average transit time, which can be an average time that a polymer translocates through a nanopore in one direction. An average transit time can be a composite of multiple transit time determinations for a polymer multipassing through a nanopore. An average transit time can be a composite of a transit time determination for a polymer translocating through a nanopore a single time with varying rates at different time points.

In certain implementations, a polymer translocates through a nanopore reader, while under tension, with a transit time of about 10 microseconds (Q s)/monomeric unit to about 100 milliseconds (ms)/monomeric unit. In certain instances, polymer translocating through the nanopore reader, while under tension, is at a transit time of about 100 □s/monomeric unit to about 10 ms/monomeric unit. In certain implementations, a polymer can translocate through a nanopore reader, while under tension, with a transit time of approximately about 100 μs/monomeric unit, 200 μs/monomeric unit, 300 μs/monomeric unit, 400 μs/monomeric unit, 500 μs/monomeric unit, 600 μs/monomeric unit, 700 μs/monomeric unit, 800 μs/monomeric unit 900 μs/monomeric unit, 1 ms/monomeric unit, 2 ms/monomeric unit, 3 ms/monomeric unit, 4 ms/monomeric unit, 5 ms/monomeric unit, 6 ms/monomeric unit, 7 ms/monomeric unit, 8 ms/monomeric unit, 9 ms/monomeric unit, 10 ms/monomeric unit, 20 ms/monomeric unit, 40 ms/monomeric unit, 50 ms/monomeric unit, 60 ms/monomeric unit, 70 ms/monomeric unit, 80 ms/monomeric unit, 90 ms/monomeric unit, 100 ms/monomeric unit.

One of the criteria used to select a translocation velocity is current signal measurement bandwidth of a data acquisition device. As a polymer translocates through a reader, each monomer unit can reside in the sensing zone for an amount of time determined by the length of the monomer unit, the length of the sensing zone, and the translocation velocity. A translocation velocity can be selected such that the nanopore reader ionic current blocking signal of each monomer is sampled within the sensing zone of the nanopore reader with at least one sample, and often multiple samples (for example, 3 or more samples, 5 or more samples, 15 of more samples, 100 or more samples, and the like) based on the bandwidth of the data acquisition device (represented by its frequency low-pass filter and/or the sampling rate). In certain embodiments, the ionic current signal of each monomer unit is sampled with about 3 measurement samples to about 500 measurement samples. In certain implementations, the number of samples per monomer unit can be 1 sample, 2 samples, 3 samples, 5 samples, 10 samples, 20 samples, 50 samples, 100 samples, 200 samples, 500 samples, 1000 samples, 10,000 samples, or 100,000 samples. In certain implementations, the data acquisition bandwidth can be reduced to reduce the noise in the current measurement and increase the signal to noise ratio. Additionally, or alternatively, the signal to noise ratio can be improved by collecting and averaging more samples per monomer unit, which increases the signal to noise ratio of the measurement by a factor proportional to the square root of the number of samples per monomer unit. In some embodiments, the translocation velocity can be changed dynamically based on triggering events, as addressed herein. For instance, the velocity can be increased or decreased when detecting the transition between an adapter and the target polymer sequence, or the velocity can be increased or decreased based on the measured signal to noise ratio of the polymer current response in order to control the number of current signal measurement samples collected for each monomer unit and averaged to produce the desired measurement signal to noise ratio.

### Polymer Sequence Determination

In certain implementations, a polymer sequence is determined from a current vs. time recording, such as a current-as-a-function-of-time signature, of a polymer translocating through a nanopore. Hidden Markov Model methods, Machine Learning methods, Trained Neural Networks, Algorithms, and/or Artificial Intelligence often are used to determine the identity of monomeric combinations passing through the sensing zone or sensing zones of the nanopore reader, when overlapping reading frames are identified (i.e., these methods deconvolute the reads of current vs. time into monomeric calls).⁵⁴⁻⁵⁷ For multipassing or rereading of the polymer strand, after the sequence of the first read is assessed, the resultant sequence can be iteratively refined/optimized by use of statistics with each additional producing a consensus sequence with the highest probability of yielding all associated reads, with accuracy improving as a function of rereads. Additionally, a subset or all of the current vs. time recordings can be averaged to yield a single current vs. time recording, which can then be deconvoluted into monomeric calls.⁵⁸⁻⁶⁰

### Certain Implementations

The following implementations A01-Q5 are provided as examples of implementations and are not limiting.

A01. A method for translocating a polymer through a nanopore, comprising:
contacting a polymer-magnetic particle conjugate with a system comprising a chip, wherein:
   the polymer of the conjugate comprises a proximal end and a distal end,
   the proximal end of the polymer is attached to the magnetic particle,
   the chip comprises a nanopore disposed in a membrane,
   the nanopore comprises an orifice smaller than the magnetic particle,
   the system comprises a magnet disposed in a cis orientation relative to the nanopore,
   the system comprises a chamber disposed in a trans orientation relative to the nanopore, and
   the magnetic particle is, or the membrane is, or the magnetic particle and the membrane are, passivated.

A01.1. The method of embodiment A01, wherein non-specific adsorption of the magnetic particle to the membrane is reduced compared to a system in which the magnetic particle and membrane are not passivated.

A01.2. The method of embodiment A01 or A01.1, wherein the membrane is an amphiphilic molecule-containing and/or lipid-containing membrane.

A01.3. The method of any one of embodiments A01-A01.2, wherein the magnetic particle is associated with a passivating agent.

A01.4. The method of embodiment A01.3, wherein the passivating agent is a surface-active passivating agent.

A01.5. The method of embodiment A01.4, wherein the surface-active passivating agent adsorbs to the magnetic particle surface.

A01.6. The method of embodiment A01.4 or A01.5, wherein the surface-active passivating agent is not covalently attached to a magnetic particle.

A01.7. The method of embodiment A01.4 or A01.5, wherein the surface-active passivating agent is not associated with a magnetic particle via an affinity tag.

A01.8. The method of any one of embodiments A01.4-A01.7, wherein the surface-active passivating agent is a surfactant.

A01.9. The method of embodiment A01.8, wherein the surfactant is a detergent or polymer.

A01.10. The method of embodiment A01.9, wherein the surfactant is chosen from sodium dodecyl sulfate (SDS), Tween-20, Triton X-100, octyl glucoside, digitonin, phospholipids, and pluronic block copolymers.

A01.11. The method of any one of embodiments A01.4-A01.7, wherein the surface-active passivating agent is a polypeptide.

A01.12. The method of embodiment A01.11, wherein the polypeptide is an albumin.

A01.13. The method of embodiment A01.12, wherein the polypeptide is bovine serum albumin (BSA).

A01.14. The method of any one of embodiments A01.4-A01.13, wherein the magnetic particle is contacted with the passivating agent for a time period before, or after, or before and after, the conjugate is contacted with the system.

A01.15. The method of embodiment A01.14, wherein the passivating agent is at a concentration of about 0.1 μM to about 1000 μM.

A01.16. The method of embodiment A01.14 or A01.15, wherein the time period is 1 minute to 24 hours.

A01.17. The method of embodiment A01.3, wherein the passivating agent is attached to the magnetic particle by a covalent bond or by an affinity tag.

A01.18. The method of embodiment A01.17, wherein the passivating agent is ionic, zwitterionic, polar, or a polymeric group.

A01.19. The method of embodiment A01.18, wherein the passivating agent is chosen from a polyethylene glycol (PEG) polymer, poly(2-methyl-2-oxazoline) polymer (PMOXA), hydroxyl, betaine, nitrile, carboxylate, methyl amide, amine, polypeptide, polystyrene sulfonate, and polyacrylate.

A01.20. The method of any one of embodiments A01.17-A01.19, wherein the passivating agent is attached to the magnetic particle by a covalent bond.

A01.21. The method of embodiment A01.20, wherein the covalent bond is formed between a reactive functional group attached to the passivating agent and a complementary functional group attached to the magnetic particle.

A01.22. The method of embodiment A01.21, wherein the reactive functional group and the complementary functional group are chosen from carboxyl/amine, NHS/amine, maleimide/thiol, thiol/thiol, amine/aldehyde, hydroxylamine/aldehyde, and alkyne/azide.

A01.23. The method of embodiment A01.22, wherein the reactive functional group and the complementary functional group is carboxyl/amine and the covalent bond is formed by reaction with a carbodiimide.

A01.24. The method of embodiment A01.23, wherein the carbodiimide is 1-ethyl-3-[3-dimethylaminopropyl]carbodiimide (EDC).

A01.26. The method of embodiment A01.25, wherein an affinity tag is attached to the passivating agent and a complementary affinity tag is attached to the magnetic particle.

A01.27. The method of embodiment A01.26, wherein the affinity tag and the complementary affinity tag are chosen from biotin/streptavidin, biotin/neutravidin, antigen/antibody, antigen/antibody fragment and polynucleotide/complimentary polynucleotide.

A01.28. The method of any one of embodiments A01.19-A01.27, wherein the passivating agent is a PEG polymer.

A01.29. The method of embodiment A01.28, wherein the PEG polymer has a molecular weight of about 0.1 kDa to about 100 kDa, or about 1 kDa to about 10 kDa, or about 5 kDa.

A01.30. The method of any one of embodiments A01-A01.29, wherein the membrane is passivated.

A01.31. The method of embodiment A01.30, wherein the membrane is a planar lipid bilayer (PLB).

A01.32. The method of embodiment A01.31, wherein the membrane includes a headgroup region containing lipid headgroups.

A01.33. The method of embodiment A01.32, wherein lipids in the PLB contain a headgroup selected to reduce an attractive interaction between the PLB and the magnetic particle.

A01.34. The method of embodiment A01.33, wherein the PLB contains lipids containing a phosphatidyl serine headgroup.

A01.35. The method of embodiment A01.34, wherein the PLB contains one or more of phosphatidylcholine (PC), phosphatidylethanolamine (PE), phosphatidylglycerol (PG), phosphatidic acid (PA) and phosphatidyl serine (PS).

A01.36. The method of embodiment A01.32, wherein lipids in the PLB contain a headgroup associated with the passivating agent.

A01.37. The method of embodiment A01.36, wherein the passivating agent comprises PEG, PMOXA, hydroxyl, betaine, nitrile, carboxylate, methyl amide, amine, polypeptide, polystyrene sulfonate, polysaccharide or a polyacrylate group.

A01.38. The method of embodiment A01.36, wherein the passivating agent is a surface-active passivating agent.

A01.39. The method of embodiment A01.38, wherein the surface-active passivating agent comprises a hydrophobic component that is capable associating with a hydrophobic region of the PLB, and a hydrophilic component that is capable of associating with remains the headgroup region.

A01.40. The method of embodiment A01.38 or A01.39, wherein the surface-active passivating agent is a surfactant.

A01.41. The method of embodiment A01.40, wherein the surfactant comprises sodium dodecyl sulfate (SDS), Tween-20, Triton X-100, octyl glucoside, digitonin, a phospholipid, a block copolymer, or a polymer covalently attached to a phospholipid or cholesterol.

A01.42. The method of any one of embodiments A01.31-A01.41, wherein the PLB comprises a PEG headgroup modified phospholipid.

A01.43. The method of embodiment A01.42, wherein the PEG headgroup modified phospholipid is 1,2-dioleoyl-glycerophosphoethanolamine.

A01.44. The method of embodiment A01.42 or A01.43, wherein the PEG headgroup modified phospholipid is contacted with a PLB in the system for a time period.

A01.45. The method of embodiment A01.44, wherein the PEG headgroup modified phospholipid is contacted with the PLB before or after the nanopore is inserted in the PLB.

A01.46. The method of embodiment A01.44 or A01.45, wherein the PEG headgroup modified phospholipid is at a concentration of about 0.1 μM to about 1000 μM.

A01.47. The method of any one of embodiments A01.44-A01.46, wherein the time period is about 1 second to about 24 hours.

A01.48. The method of any one of embodiments A01.42-A01.47, wherein PEG has a molecular weight of about 0.1 kDa to about 100 kDa, or about 1 kDa to about 10 kDa, or about 5 kDa.

A01.49. The method of any one of embodiments A01-A01.48, wherein the magnetic particle and the membrane are passivated.

A01.50. The method of embodiment A01.49, wherein the magnetic particle comprises a PEG and the membrane comprises a PEG, and optionally the PEG has a molecular weight of about 0.1 kDa to about 100 kDa, or about 1 kDa to about 10 kDa, or about 5 kDa.

A01.51. The method of any one of embodiments A01-A01.50, wherein after contacting the polymer-magnetic particle conjugate with the system as part (a):
(b) exerting an electrophoretic and/or electroosmotic force on the conjugate, in a trans direction, sufficient to dispose a portion of the polymer in the nanopore;
(c) after (b), exerting (i) a magnetic force on the conjugate in a cis direction and (ii) an electrophoretic and/or electroosmotic force on the conjugate in a trans direction, thereby exerting a net force between the electrophoretic and/or electroosmotic force and the magnetic force on the conjugate, wherein the net force translocates the polymer of the conjugate in the cis direction.

A02. A method for translocating a polymer through a nanopore, comprising:
contacting a polymer-magnetic particle conjugate with a system comprising a chip, wherein:
   the polymer of the conjugate comprises a proximal end and a distal end,
   the proximal end of the polymer is attached to the magnetic particle,
   the chip comprises a nanopore disposed in a membrane,
   the nanopore comprises an orifice smaller than the magnetic particle,
   the system comprises a magnet disposed in a cis orientation relative to the nanopore,
   the system comprises a chamber disposed in a trans orientation relative to the nanopore, and
   the membrane is a crosslinked membrane.

A02.1. The method of embodiment A02, wherein the crosslinked membrane is crosslinked before contacting he conjugate with the system.

A02.2. The method of embodiment A02 or A02.1, wherein the crosslinked membrane is crosslinked after inserting the nanopore in the membrane.

A02.3. The method of any one of embodiments A02-A02.2, wherein the crosslinked membrane optionally is a bilayer membrane, lipid membrane, black lipid membrane, block copolymer membrane, diblock copolymer membrane, dual block copolymer membrane, and/or triblock copolymer membrane; optionally comprises one or more types of amphiphilic molecules; optionally comprises one or more types of block polymers, diblock polymers, dual block polymers and/or triblock polymers; optionally comprises one or more types of surfactants; optionally comprises one or more types of lipids; and optionally comprises one or more types of polypeptides.

A02.4. The method of embodiment A02.3, wherein the amphiphilic molecules comprise one or more of lipids, block copolymers, diblock copolymers, dual block copolymers, triblock copolymers, fatty acids and surfactants.

A02.5. The method of embodiment A02.3, wherein the lipids comprise phospholipids.

A02.6. The method of any one of embodiments A02.3-A02.5, wherein the crosslinked membrane is a planar lipid bilayer (PLB).

A02.7. The method of any one of embodiments A02.3-A02.6, wherein crosslinked membrane comprises covalent bonds linking adjacent amphiphilic molecules in the membrane.

A02.8. The method of any one of embodiments A02.3-A02.7, wherein crosslinked membrane comprises polymerized reactive monomers.

A02.9. The method of embodiment A02.8, wherein the reactive monomers are embedded in the membrane and form extended polymers.

A02.10. The method of any one of embodiments A02-A02.9, wherein the crosslinked membrane, relative to a membrane that is not crosslinked, (i) increases the mechanical stability of the membrane, (ii) increase the voltage stability of the membrane, (iii) increases the lifetime of the membrane in the system, (iv) increases the stability of the nanopore within the membrane, (v) reduces nonspecific adsorption of the conjugate, or (vi) a combination of two or more of (i), (ii), (iii), (iv) and (v).

A02.11. The method of any one of embodiments A02-A02.10, wherein the crosslinked membrane comprises amphiphilic molecules containing headgroups.

A02.12. The method of any one of embodiments A02-A02.11, wherein the crosslinked membrane is crosslinked by applying a polymer having individual subunits that interact with amphiphilic molecules in the membrane.

A02.13. The method of embodiment A02.12, wherein headgroup moieties amphiphilic molecules interact with the polymer through intermolecular interactions or the formation of chemical bonds.

A02.14. The method of embodiment A02.12 or A02.13, wherein the polymer is chosen from poly L-Lysine and actin.

A02.15. The method of any one of embodiments A02-A02.14, wherein the crosslinked membrane comprises chemically modified amphiphilic molecules containing a polymerizable reactive functional group.

A02.16. The method of embodiment A02.15, wherein the polymerizable reactive functional group is disposed within a hydrophobic region of the membrane, and links adjacent amphiphilic molecules in the hydrophobic region.

A02.17. The method of embodiment A02.16, wherein the polymerizable reactive functional group is chosen from an alkene or alkyne group.

A02.18. The method of embodiment A02.16 or A02.17, wherein the amphiphilic molecules are chosen from diacetylenic lipids and bis-dienoyl phosphatidylcholine (bis-DenPC) lipids.

A02.19. The method of embodiment A02.18, wherein the amphiphilic molecules comprise 1-palmitoyl-2,10,12-tricosadiynoyl-sn-glycero-3-phosphoethanolamine (PTPE).

A02.20. The method of any one of embodiments A02.15-A02.19, wherein the polymerizable reactive functional group is disposed on headgroups of amphiphilic molecules, and links adjacent amphiphilic molecules.

A02.21. The method of embodiment A02.20, wherein the polymerizable reactive functional group is chosen from an alkyne, acrylate, methacrylate and alkene group.

A02.22. The method of any one of embodiments A02-A02.21, wherein the crosslinked membrane is crosslinked by dissolving hydrophobic reactive monomers in the membrane and initiating a chemical reaction to link the monomers together to form an extended polymer within the membrane.

A02.23. The method of embodiment A02.22, wherein the hydrophobic reactive monomers are dissolved in a hydrophobic region of the membrane.

A02.24. The method of embodiment A02.22 or A02.23, wherein the hydrophobic reactive monomers are chosen from styrene, divinylbenzene, butyl methacrylate and ethylene glycol dimethacrylate.

A02.25. The method of any one of embodiments A02.22-A02.24, wherein the chemical reaction is radical polymerization initiated by a redox stimulus.

A02.26. The method of embodiment A02.25, wherein the stimulus is ultraviolet light.

A02.27. The method of embodiment A02.26, wherein the ultraviolet light is of a wavelength of about 200 nm to about 260 nm.

A02.28. The method of embodiment A02.25, wherein the stimulus is addition of a photoinitiator and exposing the photoinitiator to light corresponding to an absorption wavelength of the photoinitiator.

A02.29. The method of embodiment A02.28, wherein the photoinitiator is chosen from benzoyl peroxide, azobisisobutyronitrile (AIBN), 2,2-diethoxy acetophenone (DEAP) and 2,4,6-trimethylbenzoyldiphenyl phosphine oxide (TPO).

A02.30. The method of embodiment A02.25, wherein the stimulus is addition of potassium persulfate, sodium metabisulfite or ferrous sulfate.

A02.31. The method of any one of embodiments A02.15-A02.30, comprising:
forming a membrane by mixing 1,2-diphytanoyl-sn-glycero-3-phosphocholine (DPhPC) and PTPE diacetylenic lipid;
inserting a nanopore into the membrane; and
crosslinking the PTPE diacetylenic lipid in the membrane by exposing the membrane to light.

A02.32. The method of embodiment A02.31, wherein the DPhPC and PTPE diacetylenic lipid are at a ratio of about 1:1 by mass.

A02.33. The method of embodiment A02.31 or A02.32, wherein the light is of a wavelength of about 254 nm light.

A02.34. The method of any one of embodiments A02.31-A02.33, wherein the membrane is exposed to light for about 5 minutes.

A02.35. The method of any one of embodiments A02-A02.34, wherein the magnetic particle, or the membrane, or the magnetic particle and the membrane, are passivated.

A02.36. The method of embodiment A02.35, wherein the magnetic particle, or the membrane, or the magnetic particle and the membrane, are passivated before, or after, or before and after, the membrane is crosslinked.

A02.37. The method of any one of embodiments A02-A02.36, wherein after contacting the polymer-magnetic particle conjugate with the system as part (a):
(b) exerting an electrophoretic and/or electroosmotic force on the conjugate, in a trans direction, sufficient to dispose a portion of the polymer in the nanopore;
(c) after (b), exerting (i) a magnetic force on the conjugate in a cis direction and (ii) an electrophoretic and/or electroosmotic force on the conjugate in a trans direction, thereby exerting a net force between the electrophoretic and/or electroosmotic force and the magnetic force on the conjugate, wherein the net force translocates the polymer of the conjugate in the cis direction.

A03. A method for translocating a polymer through a nanopore, comprising:
(a) contacting a polymer-magnetic particle conjugate with a system comprising a chip, wherein:
   the polymer of the conjugate comprises a proximal end and a distal end,
   the proximal end of the polymer is attached to the magnetic particle,
   the chip comprises a nanopore disposed in a membrane,
   the nanopore comprises an orifice smaller than the magnetic particle,
   the system comprises a magnet disposed in a cis orientation relative to the nanopore,
   the system comprises a chamber disposed in a trans orientation relative to the nanopore, and
   the chamber comprises a hard stop agent;
(b) exerting an electrophoretic and/or electroosmotic force on the conjugate, in a trans direction, sufficient to dispose a portion of the polymer in the chamber, under conditions in which the hard stop agent associates with a distal portion or distal terminus of the polymer in the chamber, thereby forming a distal hard stop structure, wherein a hydrodynamic diameter of the hard stop structure is larger than a constriction region diameter of the nanopore.

A03.1. The method of embodiment A03, wherein the magnetic particle, the polymer and the hard stop structure form a pseudorotaxane structure.

A03.2. The method of embodiment A03 or A03.1, wherein the magnetic particle is in association with the nanopore under the conditions in which the hard stop agent associates with a portion of the polymer in the chamber.

A03.3. The method of embodiment A03 or A03.1, wherein the magnetic particle is not in association with the nanopore under the conditions in which the hard stop agent associates with a portion of the polymer in the chamber.

A03.4. The method of any one of embodiments A03-A03.3, wherein the distal portion of the polymer is disposed about 1 to about 1,000 contiguous nucleotides from the distal terminus of the polymer.

A03.5. The method of any one of embodiments A03-A03.4, wherein the hard stop agent is associated with the polymer by a covalent bond, or non-covalent interaction, or covalent bond, and non-covalent interaction.

A03.6. The method of any one of embodiments A03-A03.5, wherein:
the polymer comprises an affinity tag linked to the distal portion or distal end of the polymer; and
the affinity tag is capable of associating with the hard stop agent or a complementary affinity tag linked to the hard stop agent.

A03.7. The method of any one of embodiments A03-A03.6, wherein the hard stop agent comprises an affinity tag.

A03.8. The method of embodiment A03.7, wherein the affinity tag has a hydrodynamic diameter larger than the diameter of the constriction region of the nanopore.

A03.9. The method of embodiment A03.7 or A03.8, wherein the affinity tag is chosen from streptavidin, neutravidin, antibody, antibody fragment, nucleic acid, DNA, RNA and artificial nucleic acid.

A03.10. The method of any one of embodiments A03.7-A03.9, wherein and the polymer comprises a complementary affinity tag.

A03.11. The method of embodiment A03.10, wherein the complementary affinity tag of the polymer can enter and traverse the nanopore.

A03.12. The method of embodiment A03.10 or A03.11, wherein the complementary affinity tag of the polymer is chosen from biotin, antigen, and polynucleotide capable of hybridizing to complementary polynucleotide present in the hard stop agent.

A03.13. The method of any one of embodiments A03-A03.12, wherein:
the hard stop agent comprises a reactive group and the polymer comprises a complementary reactive group; and
the reactive group and complementary reactive group are capable of forming a covalent bond.

A03.14. The method of embodiment A03.13, wherein the reactive group and complementary reactive group are chosen from amine/carboxylate, alkyne/azide and thiol/maleimide.

A03.15. The method of any one of embodiments A03-A03.14, wherein the hard stop agent comprises a branched polymer, a nanoparticle, a protein, or a folded DNA structure.

A03.15. The method of embodiment A03.14, wherein the folded DNA structure is chosen from an i-motif, a G-quadruplex, a DNA duplex, a DNA-RNA duplex, a RNA-RNA duplex, and a DNA origami structure.

A03.16. The method of any one of embodiments A03.9-A03.15, wherein the hard stop agent comprises an affinity tag and a reactive group and the polymer comprises a complementary reactive group.

A03.17. The method of embodiment A03.16, wherein and the polymer comprises a complementary affinity tag.

A03.18. The method of embodiment A03.16 and A03.17, wherein the hard stop agent binds to the polymer or complementary affinity tag via the affinity tag, and the reactive group and complementary reactive group react and form a covalent bond associating the hard stop agent to the polymer.

A03.19. The method of any one of embodiments A03-A03.18, wherein the hard stop agent is a hard stop oligonucleotide comprising a polynucleotide complementary and capable of hybridizing to a polynucleotide of the polymer.

A03.20. The method of embodiment A03.19 or A03.20, wherein the oligonucleotide or polynucleotide of the capture molecule is about 10 to about 200 contiguous nucleotides in length.

A03.21. The method of embodiments A03.19 or A03.20, wherein the hard stop oligonucleotide, or polynucleotide complementary and capable of hybridizing to a polynucleotide of the polymer, comprises one or more locked nucleotides.

A03.22. The method of any one of embodiments A03.19-A03.21, comprising joining an oligonucleotide to the polymer, wherein the oligonucleotide joined to the polymer comprises the polynucleotide complementary to, and capable of hybridizing to, a polynucleotide in the hard stop oligonucleotide.

A03.23. The method of embodiment A03.22, wherein the oligonucleotide joined to the polymer comprises one or more locked nucleotides.

A03.24. The method of any one of embodiments A03.19-A03.23, wherein the polynucleotide in the polymer complementary to, and capable of hybridizing to, the polynucleotide in the hard stop oligonucleotide, is at the distal terminus of the hard stop oligonucleotide, or is incorporated within the polymer upstream of the target polymer distal end at a distance from the proximal end of the polymer whereby the entirety of the capture polynucleotide resides in the chamber.

A03.25. The method of any one of embodiments A03-A03.24, wherein the hard stop agent is at a concentration in the compartment of about 100 μM to about 1 mM.

A03.26. The method of embodiment A03.25, wherein the hard stop agent is at a concentration in the compartment of about 100 nM to about 50 μM.

A03.27. The method of any one of embodiments A03.10-A03.26, wherein:
the hard stop agent comprises or consists of an affinity tag and the polymer comprises a complementary affinity tag, and
the affinity tag is streptavidin or neutravidin and the complementary affinity tag is biotin disposed on the distal terminus of the polymer, or
the affinity tag is a polynucleotide and the complementary affinity tag is a complementary polynucleotide capable of hybridizing to the affinity tag polynucleotide.

A03.28. The method of any one of embodiments A03-A03.5, wherein the polymer does not include a complementary affinity tag.

A03.29. The method of any one of embodiments A03-A03.28, wherein a voltage bias permitting association of the polymer in the nanopore, but not translocate through the nanopore until the magnetic particle contacts the nanopore, is held for a period of time as part of the conditions in which the hard stop agent associates with a portion of the polymer in the chamber.

A03.30. The method of any one of embodiments A03-A03.28, wherein a voltage bias permitting the polymer to translocate through the nanopore until the magnetic particle contacts the nanopore, is held for a period of time as part of the conditions in which the hard stop agent associates with a portion of the polymer in the chamber.

A03.31. The method of any one of embodiments A03-A03.30, comprising (c) after (b), exerting (i) a magnetic force on the conjugate in a cis direction and (ii) an electrophoretic and/or electroosmotic force on the conjugate in a trans direction, thereby exerting a net force between the electrophoretic and/or electroosmotic force and the magnetic force on the conjugate, wherein the net force translocates the polymer of the conjugate in the cis direction.

A04. The method of any one of embodiments A01-A01.51, A02-A02.37 and A03-A03.31, comprising a combination of two or three of the following (i), (ii) and (iii):
(i) the magnetic particle is, or the membrane is, or the magnetic particle and the membrane are, passivated according to any one of embodiments A01-A01.51;
(ii) the membrane is a crosslinked membrane according to any one of embodiments A02-A02.37; and
(iii) the chamber comprises a hard stop agent and/or the conjugate comprises a distal hard stop structure according to any one of embodiments A03-A03.31.

A05. The method of any one of embodiments A01.51, A02.37 and A03.31, comprising (d), after (c), iteratively modulating the net force on the conjugate and thereby iteratively translocating the polymer of the conjugate in opposing directions through the nanopore, wherein in an iteration the polymer of the conjugate translocates through the nanopore in a direction different than the direction in a preceding iteration.

A1. A method for iteratively translocating a polymer through a nanopore in opposing directions, comprising:
(a) contacting a polymer-magnetic particle conjugate with a system comprising a chip, wherein:
   the polymer of the conjugate comprises a proximal end and a distal end,
   the proximal end of the polymer is attached to the magnetic particle,
   the chip comprises a nanopore, and
   the nanopore comprises an orifice smaller than the magnetic particle;
(b) after (a), exerting an electrophoretic and/or electroosmotic force on the conjugate, in a trans direction, sufficient to dispose a portion of the polymer in the nanopore;
(c) after (b), exerting (i) a magnetic force on the conjugate in a cis direction and (ii) an electrophoretic and/or electroosmotic force on the conjugate in a trans direction, thereby exerting a net force between the electrophoretic and/or electroosmotic force and the magnetic force on the conjugate; and
(d) after (c), iteratively modulating the net force on the conjugate, wherein in an iteration the polymer of the conjugate translocates through the nanopore in a direction different than the direction in a preceding iteration.

A2. A method for translocating a polymer through a nanopore, comprising:
(a) contacting a polymer-magnetic particle conjugate with a system comprising a chip, wherein:
   the polymer of the conjugate comprises a proximal end and a distal end,
   the proximal end of the polymer is attached to the magnetic particle,
   the chip comprises a nanopore, and
   the nanopore comprises an orifice smaller than the magnetic particle;
(b) after (a), exerting an electrophoretic and/or electroosmotic force on the conjugate, in a trans direction, sufficient to dispose a portion of the polymer in the nanopore;
(c) after (b), exerting (i) a magnetic force on the conjugate in a cis direction and (ii) an electrophoretic and/or electroosmotic force on the conjugate in a trans direction, thereby exerting a net force between the electrophoretic and/or electroosmotic force and the magnetic force on the conjugate, wherein the net force translocates the polymer of the conjugate in the cis direction.

A3. The method of any one of embodiments A01-A2, wherein the chip comprises an electrode disposed in a trans position relative to the nanopore for application of a voltage bias across the nanopore,

A4. The method of any one of embodiments A01-A3, wherein:
the system comprises a magnet, and
the magnet is disposed in a cis position relative to the nanopore for application of a magnetic field at the nanopore.

A5. A method for iteratively translocating a polymer through a nanopore in opposing directions, comprising:
(a) contacting a polymer-magnetic particle conjugate with a system comprising a chip and a magnet, wherein:
   the polymer of the conjugate comprises a proximal end and a distal end,
   the proximal end of the polymer is attached to the magnetic particle,
   the chip comprises a nanopore and an electrode disposed in a trans position relative to the nanopore for application of a voltage bias across the nanopore,
   the magnet is disposed in a cis position relative to the nanopore for application of a magnetic field at the nanopore, and
   the nanopore comprises an orifice smaller than the magnetic particle;
(b) after (a), applying a voltage bias across the nanopore sufficient to capture the conjugate in the nanopore, wherein a portion of the polymer is disposed in the nanopore and the magnetic particle is disposed at a cis position outside the nanopore;
(c) after (b), applying (i) a magnetic field sufficient to attract the magnetic particle of the conjugate in a cis direction and (ii) a voltage bias across the nanopore sufficient to impart an electrophoretic and/or electroosmotic force on the conjugate in a trans direction, thereby exerting a net force between the electrophoretic and/or electroosmotic force and the magnetic force on the conjugate disposed in the nanopore; and
(d) after (c), iteratively modulating the net force on the conjugate and thereby iteratively translocating the polymer of the conjugate in opposing directions through the nanopore, wherein in an iteration the polymer of the conjugate translocates through the nanopore in a direction different than the direction in a preceding iteration.

A6. A method for translocating a polymer through a nanopore, comprising:
(a) contacting a polymer-magnetic particle conjugate with a system comprising a chip and a magnet, wherein:
   the polymer of the conjugate comprises a proximal end and a distal end,
   the proximal end of the polymer is attached to the magnetic particle,
   the chip comprises a nanopore and an electrode disposed in a trans position relative to the nanopore for application of a voltage bias across the nanopore,
   the magnet is disposed in a cis position relative to the nanopore for application of a magnetic field at the nanopore, and
   the nanopore comprises an orifice smaller than the magnetic particle;
(b) after (a), applying a voltage bias across the nanopore sufficient to capture the conjugate in the nanopore, wherein a portion of the polymer is disposed in the nanopore and the magnetic particle is disposed at a cis position outside the nanopore;
(c) after (b), applying (i) a magnetic field sufficient to attract the magnetic particle of the conjugate in a cis direction and (ii) a voltage bias across the nanopore sufficient to impart an electrophoretic and/or electroosmotic force on the conjugate in a trans direction, thereby exerting a net force between the electrophoretic and/or electroosmotic force and the magnetic force on the conjugate disposed in the nanopore, wherein the net force translocates the polymer of the conjugate in the cis direction.

A7. The method of any one of embodiments A05, A1 and A3-A5, wherein (d) comprises iteratively modifying the magnetic field, or iteratively modifying the voltage bias across the nanopore, or iteratively modifying the magnetic field and iteratively modifying the voltage bias across the nanopore.

A8. The method of any one of embodiments A05, A1, A3-A5 and A7, wherein (d) comprises:
(1) applying a first net force sufficient to translocate at least a portion of the polymer through the nanopore in the cis direction; and
(2) applying a second net force, different than the first net force, sufficient to translocate at least a portion of the polymer through the nanopore in the trans direction.

A9. The method of any one of embodiments A05, A1, A3-A5, A7 and A8, wherein (d) comprises:
(1) applying a first voltage bias sufficient to translocate at least a portion of the polymer through the nanopore in the cis direction; and
(2) applying a second voltage bias, different than the first voltage bias, sufficient to translocate at least a portion of the polymer through the nanopore in the trans direction.

A10. The method of any one of embodiments A01.51, A02.37, A03.31, A05 and A1-A9, wherein the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, under a constant magnetic force.

A11. The method of any one of embodiments A01.51, A02.37, A03.31, A05 and A1-A9, wherein the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, under a constant magnetic field.

A12. The method of any one of embodiments A01.51, A02.37, A03.31, A05 and A1-A9, wherein:
the system comprises a magnet and the magnetic field is applied by the magnet; and
the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, without modifying the magnetic field exerted by the magnet.

A13. The method of any one of embodiments A01.51, A02.37, A03.31, A05 and A1-A9, wherein:
the system comprises a magnet and the magnetic field is applied by the magnet; and
the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, without altering the position of the magnet.

A14. The method of any one of embodiments A01.51, A02.37, A03.31, A05 and A1-A9, wherein:
the system comprises a magnet and the magnetic field is applied by the magnet; and
the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, without the conjugate contacting the magnet.

A15. The method of any one of embodiments A01.51, A02.37, A03.31, A05 and A1-A9, wherein the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, with the conjugate unattached to, or unassociated with, a solid support.

A16. The method of any one of embodiments A01.51, A02.37, A03.31, A05 and A1-A9, wherein the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, with the conjugate unattached to, or unassociated with, a moveable solid support.

A16.1. The method of any one of embodiments A01.51, A02.37, A03.31, A05 and A1-A9, wherein:
the polymer translocates through the nanopore in part (c) of any one of embodiments A01.51, A02.37, A03.31, A2 and A6, or the polymer translocates through the nanopore in part (d) of any one of embodiments A05, A1, A3-A5 and A7-A9, by a motivation force; and
the motivation force consists of the net force between (i) the electrophoretic and/or electroosmotic force and (ii) the magnetic force, on the conjugate.

A16.2. The method of any one of embodiments A01.51, A02.37, A03.31, A05 and A1-A16.1, wherein a net force between (i) the electrophoretic and/or electroosmotic force and (ii) the magnetic force, on the conjugate, is modulated by modulating an electric field at the nanopore.

A16.3. The method of any one of embodiments A01.51, A02.37, A03.31, A05 and A1-A16.2, wherein a net force between (i) the electrophoretic and/or electroosmotic force and (ii) the magnetic force, on the conjugate, is modulated by modulating an electric force on the conjugate at the nanopore.

A16.4. The method of any one of embodiments A01.51, A02.37, A03.31, A05 and A1-A16.3, wherein a net force between (i) the electrophoretic and/or electroosmotic force and (ii) the magnetic force, on the conjugate, is not modulated by modulating a magnetic field at the nanopore in a system.

A16.5. The method of any one of embodiments A01.51, A02.37, A03.31, A05 and A1-A16.4, wherein a net force between (i) the electrophoretic and/or electroosmotic force and (ii) the magnetic force, on the conjugate, is not modulated by modulating a magnetic force on the conjugate at the nanopore.

A17. The method of any one of embodiments A8-A16.5, wherein (1) and (2) are in a cycle and the cycle is repeated.

A18. The method of embodiment A17, wherein (1) and (2) are in a cycle and each cycle is repeated two times to about 10,000 times.

A19. The method of any one of embodiments A01.51, A02.37, A03.31, A05 and A1-A18, wherein in (b), (c) and (d) the magnetic particle is disposed at a cis position outside the nanopore.

B1. The method of any one of embodiments A01.51, A02.37, A03-A03.31, A05 and A1-A19, wherein in (b) a capture voltage bias is applied across the nanopore sufficient to dispose a portion of the polymer in the nanopore.

B2. The method of embodiment B1, wherein the capture voltage bias is sufficient to translocate the polymer through the nanopore in the trans direction until the proximal end of the polymer is disposed at or near the nanopore.

B3. The method of embodiment B2, wherein the capture voltage bias is about 80 mV to about 300 mV.

B4. The method of embodiment B3, wherein the capture voltage bias is about 180 mV to about 220 mV.

B5. The method of embodiment B4, wherein the capture voltage bias is about 200 mV.

B6. The method of any one of embodiments B1-B5, comprising in part (b):
optionally measuring ionic current,
optionally determining a translocation velocity of the polymer through the nanopore, and
identifying a polymer capture event.

B7. The method of embodiment B6, wherein the polymer capture event comprises one or more of: a measured current, noise level, current and/or noise level for a period of time, current signature for specific monomers disposed at or near the proximal end of the polymer, stalled current associated with the magnetic particle in contact with the nanopore, current signature for one or more molecular markers disposed on the polymer, a polymer translocation velocity associated with the proximal end of the polymer disposed at or near the nanopore, and a predetermined period of time.

B8. The method of any one of embodiments B1-B7, wherein after (b) and prior to (c) a holding voltage bias is applied across the nanopore sufficient to retain a majority of the polymer in a trans position relative to the nanopore.

B9. The method of any one of embodiments B6-B8, wherein the holding voltage bias is applied after the polymer capture event is identified.

B10. The method of any one of embodiments B6-B9, wherein the magnetic force is exerted and the magnetic field is applied in (c) after the polymer capture event is identified, or after the holding voltage bias is applied, or after the polymer capture event is identified and after the holding voltage bias is applied.

B11. The method of any one of embodiments B9-B10, wherein the holding voltage bias is about 10 mV to about 250 mV.

B12. The method of embodiment B11, wherein the holding voltage bias is about 140 mV to about 180 mV.

B13. The method of embodiment B12, wherein the holding voltage bias is about 160 mV.

B13.1. The method of any one of embodiments A01.51, A02.37, A03.31, A05 and A1-A19 and B1-B13, wherein the chip comprises a chamber disposed in a trans position relative to the nanopore; the chamber includes a hard stop agent; and the capture voltage bias is, or the holding voltage bias is, or the capture voltage bias and the holding voltage bias are, held for a time sufficient for formation of the distal hard stop structure.

B14. The method of any one of embodiments A01.51, A02.37, A03.31, A05 and A1-A19 and B1-B13.1, wherein in (c) the magnetic force does not exceed the electrophoretic and/or electroosmotic force.

B15. The method of any one of embodiments A7-A19 and B1-B14, wherein in (1) the first voltage bias is an exit voltage bias that imparts an electrophoretic and/or electroosmotic force less than the magnetic force on the conjugate.

B16. The method of embodiment B15, wherein the exit voltage bias is about 10 mV to about 200 mV.

B17. The method of embodiment B16, wherein the exit voltage bias is about 100 mV to about 140 mV.

B18. The method of embodiment B17, wherein the exit voltage bias is about 120 mV.

B19. The method of any one of embodiments A7-A19 and B1-B19, wherein in (2) the second voltage bias is a reentry voltage bias that imparts an electrophoretic and/or electroosmotic force greater than the magnetic force on the conjugate.

B20. The method of embodiment B19, wherein the reentry voltage bias is about 80 mV to about 300 mV.

B21. The method of embodiment B20, wherein the reentry voltage bias is about 180 mV to about 240 mV.

B22. The method of embodiment B21, wherein the reentry voltage bias is about 200 mV.

B22.1. The method of any one of embodiments A7-A19 and B1-B22, wherein the chip comprises a chamber disposed in a trans position relative to the nanopore and the conjugate comprises a distal hard stop, and comprising in part (d) prior to part (2):
optionally measuring ionic current,
optionally determining a translocation velocity of the polymer through the
nanopore, and identifying a distal hard stop and nanopore interaction event.

B22.2. The method of embodiment B22.1, wherein the distal hard stop and nanopore interaction event is stalled current associated with the distal hard stop structure in contact with the nanopore in the chamber, or interaction in the chamber of the distal hard stop structure with the nanopore.

B22.3. The method of embodiment B22.1 or B22.2, wherein:
a distal hard stop stripping voltage bias is applied after the distal hard stop and nanopore interaction event is identified, and
the stripping voltage bias is sufficient to disassociate the hard stop agent from the polymer in the chamber and exit the polymer of the conjugate from of the chamber and the nanopore.

B22.4. The method of embodiment B22.3, wherein the stripping voltage bias is greater than 60 mV.

B22.5. The method of embodiment B22.3 or B22.4, wherein the polymer of the conjugate exiting the nanopore is a polymer exit event.

B23. The method of any one of embodiments A7-A19 and B1-B22.5, comprising in part (d) prior to part (2):
optionally measuring ionic current,
optionally determining a translocation velocity of the polymer through the nanopore, and identifying a polymer exit event.

B24. The method of embodiment B23, wherein the polymer exit event comprises one or more of: a measured current, noise level, current and/or noise level for a period of time, current signature for specific monomers disposed at or near the distal end of the polymer, current signature for one or more molecular markers disposed on the polymer, a polymer translocation velocity associated with the distal end of the polymer disposed at or near the nanopore, and a predetermined period of time.

B25. The method of embodiment B23 or B24, wherein the reentry voltage bias is applied after the polymer exit event is identified.

B25.1. The method of embodiment B22.1 or B22.2, wherein the reentry voltage bias is applied after the distal hard stop and nanopore interaction event is identified. B26. The method of any one of embodiments B19-B25.1, comprising in part (d) after part (2):
optionally measuring ionic current,
optionally determining a translocation velocity of the polymer through the nanopore, and
identifying a polymer reentry event.

B27. The method of embodiment B26, wherein the polymer reentry event comprises one or more of: a measured current, noise level, current and/or noise level for a period of time, current signature for specific monomers disposed at or near the proximal end of the polymer, stalled current associated with the magnetic particle in contact with the nanopore, current signature for one or more molecular markers disposed on the polymer, a polymer translocation velocity associated with the proximal end of the polymer disposed at or near the nanopore, and a predetermined period of time.

B28. The method of any one of embodiments A8-A19 and B1-B27, wherein part (1) and part (2) are in a cycle and the cycle is repeated one or more times.

B29. The method of embodiment B28, wherein the cycle is repeated after the polymer reentry event of embodiment B26 or B27 is identified.

B30. The method of any one of embodiments A8-A19 and B1-B29, wherein:
ionic current is measured in part (d), and
part (d) comprises, in part (2), identifying a polymer sequence-dependent current blocking signature.

B31. The method of any one of embodiments A8-A19 and B1-B30, wherein:
ionic current is measured in part (d), and
part (d) comprises, in part (1), identifying a polymer sequence-dependent current blocking signature.

B32. The method of embodiment B30 or B31, wherein:
part (1) and part (2) are in a cycle;
the cycle is repeated, thereby identifying multiple polymer sequence-dependent current blocking signatures; and
a consensus sequence for the polymer of the conjugate is determined based on the multiple polymer sequence-dependent current blocking signatures.

B33. The method of embodiment B32, wherein the consensus sequence for the polymer is determined based on:
(i) the multiple polymer sequence-dependent current blocking signatures identified in part (1), or
(ii) the multiple polymer sequence-dependent current blocking signatures identified in part (2), or
(iii) the multiple polymer sequence-dependent current blocking signatures identified in part (1) and the multiple polymer sequence-dependent current blocking signatures identified in part (2).

B34. The method of any one of embodiments B28-B33, wherein the cycle is repeated two times to about 10,000 times.

C1. The method of any one of embodiments A01-A19 and B1-B34, wherein the distal end of the polymer is single stranded.

C2. The method of embodiment C1, wherein the polymer in (a) is single stranded.

C3. The method of embodiment C1 or C2, wherein the polymer in (a) is partially double stranded.

C4. The method of embodiment C3, wherein the polymer after (b) is single stranded.

C5. The method of embodiment C4, wherein:
a first strand is disposed in the nanopore and a second strand is not disposed in the nanopore, and
the second strand is separated from the first strand in (b).

C6. The method of embodiment C5, comprising applying and holding a capture voltage bias for a period of time that is (i) not sufficient to translocate the polymer through the nanopore in the trans direction until the proximal end of the polymer is disposed at or near the nanopore, and (ii) is sufficient to induce duplex dissociation.

C7. The method of embodiment C5, comprising applying a capture voltage bias sufficient to translocate the polymer through the nanopore in the trans direction until the proximal end of the polymer is disposed at or near the nanopore, thereby dissociating the second strand from the first strand as the first strand translocates through the nanopore.

C8. The method of any one of embodiments C1-C7, wherein the polymer comprises a polynucleotide that is translocated through the nanopore and the polymer is not contacted with a chaotropic agent or denaturant in (a)-(d).

C8.1. The method of any one of embodiments C1-C7, wherein the polymer comprises a polypeptide that is translocated through the nanopore and the polymer is contacted with a surfactant and/or denaturant.

C9. The method of any one of embodiments C1-C8.1, wherein the polymer is a biological polymer.

C10. The method of embodiment C9, wherein the polymer comprises native or modified deoxyribonucleic acid (DNA), native or modified ribonucleic acid (RNA) or native or modified amino acid.

C11. The method of any one of embodiments C1-C10, wherein the polymer comprises one or more molecular markers.

D1. The method of any one of embodiments A01.51, A02.37, A03.31, A05, A1-A19, B1-B34 and C1-C11, wherein in (c) and (d) the polymer is elongated.

D2. The method of embodiment D1, wherein:
the polymer in (a) contains secondary structure and/or tertiary structure, and
the net force on the conjugate in (c) exceeds a force required to disrupt secondary structure and tertiary structure in the polymer.

D3. The method of embodiment D1 or D2, wherein:
a tensile force is exerted on the polymer in (c) and (d), and the tensile force is about 0.1 to about 200 picoNewtons (pN).

D4. The method of any one of embodiments D1-D3, wherein the tensile force is about 0.1 pN to about 100 pN.

D5. The method of embodiment D4, wherein the tensile force is about 40 pN to about 80 pN.

E1. The method of any one of embodiments A05, A1-A19, B1-B34, C1-C11 and D1-D5, wherein the polymer translocates through the nanopore in (d) at a velocity based on a transit time of about 10 microseconds per polymer monomeric unit to about 100 milliseconds per polymer monomeric unit.

E1.1. The method of embodiment E1, wherein the transit time is about 100 microseconds per polymer monomeric unit to about 10 milliseconds per polymer monomeric unit.

E2. The method of any one of embodiments A7-A19, B1-B34, C1-C11, D1-D5, E1 and E1.1, wherein the polymer translocates through the nanopore in a trans-to-cis direction after the first voltage bias is applied in part (1) at a velocity based on a transit time of about 10 microseconds per polymer monomeric unit to about 100 milliseconds per polymer monomeric unit.

E3. The method of any one of embodiments A7-A19, B1-B34, C1-C11, D1-D5 and E1-E2, wherein the polymer translocates through the nanopore in a cis-to-trans direction after the second voltage bias is applied in part (2) at a velocity based on a transit time of about 10 microseconds per polymer monomeric unit to about 100 milliseconds per polymer monomeric unit.

E4. The method of embodiment E3, wherein the velocity after the first voltage bias is applied in part (1) and the velocity after the second voltage bias is applied in part (2) are the same or different.

E5. The method of any one of embodiments E1-E4, wherein:
ionic current is measured in part (d);
part (d) comprises, in part (1) and/or part (2), determining a polymer sequence-dependent current blocking signature.

E6. The method of embodiment E5, wherein part (d) comprises determining two or more signatures in part (1) or part (2).

F1. The method of any one of embodiments A01-A19, B1-B34, C1-C11, D1-D5 and E1-E6 wherein the system comprises a circuit comprising electrodes, an amplifier in connection with at least one of the electrodes, a data acquisition (DAQ) system in connection with the amplifier, a bias module in connection with the amplifier, and a control module in connection with the bias module and the DAQ system.

F2. The method of embodiment F1, wherein the control module comprises one or more of: a field-programmable gate array (FPGA), a microprocessor, memory, a microcontroller, a computer, an application specific integrated circuit (ASIC), and a fixed hardware circuit.

F3. The method of embodiment F1 or F2, wherein the amplifier is a resistive feedback amplifier.

F4. The method of embodiment F1 or F2, wherein the amplifier is a capacitive feedback amplifier.

F5. The method of any one of embodiments F1-F4, wherein the circuit is an application-specific integrated circuit (ASIC).

F6. The method of embodiment F5, wherein the ASIC comprises the DAQ system, the control module, the amplifier(s) and the bias module(s).

F7. The method of any one of embodiments F1-F6, wherein the connection between the amplifier and at least one of the electrodes comprises a current-to-voltage converter circuit.

F8. The method of any one of embodiments F1-F7, wherein the DAQ system comprises at least one analog-to-digital converter (ADC).

F9. The method of any one of embodiments F1-F8, wherein the electrodes comprise a working electrode.

F10. The method of embodiment F9, wherein the at least one ADC is configured to acquire an analog signal originating from the working electrode.

F11. The method of any one of embodiments F1-F10, wherein the DAQ system comprises at least one digital-to-analog converter (DAC).

F12. The method of embodiment F11, wherein the at least one DAC is configured to produce a voltage bias level that is applied to the working electrode through the amplifier.

F13. The method of embodiment F11 or F12, wherein the at least one DAC is configured to produce preset voltage bias levels.

F13.1. The method of embodiment F12 or F13, wherein the voltage bias levels are filtered to a noise level of 20 nV/rtHz or less.

F14. The method of any one of embodiments F1-F13.1, wherein the DAQ system comprises an analog signal condition circuit.

F14.1. The method of embodiment F14, wherein the analog signal condition circuit comprises one or more of: a filter, gain stage and attenuator.

F14.2. The method of any one of embodiments F1-F13.1, wherein the DAQ system comprises an integrator, differentiator or difference amplifier.

F15. The method of any one of embodiments F1-F14.2, wherein the electrodes comprise a working electrode in proximity to the nanopore and a reference electrode.

F16. The method of embodiment F15, wherein the working electrode is disposed in a trans position relative to the nanopore and the reference electrode is disposed in a cis position relative to the nanopore.

F17. The method of embodiment F15 or F16, wherein the chip comprises multiple sensor sites, a nanopore at each of the sensor sites and a working electrode in proximity to each nanopore.

F18. The method of embodiment F17, wherein the bias module of the circuit comprises a multiplexer circuit.

F19. The method of embodiment F18, wherein the multiplexer circuit is configured to select preset voltage bias levels produced by the DAC.

F20. The method of embodiment F19, wherein the preset voltage bias levels are selected by the multiplexer circuit manually or according to a routine managed by the control module.

F21. The method of any one of embodiments F18-F20, wherein the system comprises multiple amplifiers and each amplifier is paired with a dedicated multiplexer circuit.

F22. The method of any one of embodiments F1-F21, wherein the control module is in connection with the ADC(s) and the DAC(s).

F23. The method of embodiment F22, wherein the control module is configured to receive electrode current measurements from the ADC(s).

F24. The method of any one of embodiments F1-F23, wherein the control module is configured to detect one or more of a polymer capture event, polymer exit event and polymer reentry event.

F25. The method of embodiment F24, wherein the control module is configured to detect one or more of the polymer capture event, polymer exit event and polymer reentry event in quasi-real time.

F26. The method of embodiment F24 or F25, wherein one or more of the polymer capture event, polymer exit event and polymer reentry event occur at predetermined set points stored in the control module.

F27. The method of any one of embodiments F24-F26, wherein the control module is configured to transmit to the DAC(s) one or more of a capture voltage bias, holding voltage bias, exit voltage bias and reentry voltage bias levels.

F28. The method of any one of embodiments F23-F26, wherein the control module is configured to transmit to the multiplexer circuit a change in the voltage bias level to (i) the holding voltage bias level after detecting the polymer capture event, or (ii) the exit voltage bias level after detecting the polymer capture event, or (iii) the reentry voltage bias level after detecting the polymer exit event, or (iv) the exit voltage bias level after detecting the polymer reentry event, or a combination of two, three or all of (i), (ii), (iii) and (iv).

F29. The method of any one of embodiments F1-F28, wherein the control module is configured to activate or deactivate the magnet.

F30. The method of embodiment F29, wherein the control module is configured to activate the magnet after detecting the capture event.

F31. The method of embodiment F29 or F30, wherein the control module directly activates or deactivates the magnet.

F32. The method of embodiment F29 or F30, wherein the control module activates or deactivates the magnet by a switch.

F33. The method of any one of embodiments F29-F32, wherein the control module activates or deactivates the magnet via a digital line from the controller to the magnet or to the switch.

F34. The method of any one of embodiments F24-F33, wherein one or more of the polymer capture event, polymer exit event and polymer reentry event comprise a current measurement.

F35. The method of embodiment F34, wherein the detecting a trigger event comprises detecting a current measurement.

F36. The method of embodiment F35, wherein the current measurement is a high bandwidth current measurement.

F37. The method of embodiment F36, wherein the current measurement is at about 100 kiloHertz (kHz) to about 250 kHz.

F38. The method of embodiment F36, wherein the current measurement is at about 25 kHz to about 75 kHz.

F39. The method of embodiment F36, wherein the current measurement is at about 1 kHz to about 10 kHz (for example, for over 100 channels).

F40. The method of any one of embodiments F36-F39, wherein one or more amplifiers in the system each have a bandwidth of about 10 Hz to about 1 kHz and the system implements one or more software routines that permit a high bandwidth current measurement.

F41. The method of any one of embodiments F1-F40, wherein the voltage bias applied at the nanopore is changed in a time frame of about 0.01 milliseconds (ms) to about 5 ms.

F42. The method of any one of embodiments F1-F40, wherein the voltage bias applied at the nanopore is changed in a time frame of about 0.05 ms to about 1 ms.

F43. The method of any one of embodiments F1-F40, wherein the voltage bias applied at the nanopore is changed in a time frame of about 0.075 ms to about 0.25 ms.

F44. The method of any one of embodiments F1-F40, wherein the voltage bias applied at the nanopore is changed in a time frame of about 0.1 ms.

F45. The method of any one of embodiments F1-F44, wherein the bias module produces a filtered preset voltage bias level having a noise of about 20 nV/rtHz or less after filtering.

G1. The method of any one of embodiments A01-A19, B1-B34, C1-C11, D1-D5, E1-E6 and F1-F45, wherein the chip comprises a substrate and a layer disposed on the substrate.

G2. The method of embodiment G1, wherein the substrate comprises a material chosen from quartz, silica, glass, sapphire, printed circuit board laminate, polycarbonate and polytetrafluoroethylene (PTFE).

G3. The method of embodiment G2, wherein the substrate comprises fused quartz and/or fused silica.

G4. The method of any one of embodiments G1-G3, wherein the substrate comprises a thickness of about 0.1 mm to about 2.0 mm.

G5. The method of any one of embodiments G1-G4, wherein the layer comprises a thickness of about 0.1 micrometers to about 500 micrometers.

G6. The method of ay one of embodiments G1-G5, wherein the layer comprises one or more of epoxy-based negative photoresist, SU-8, polyimide, parylene, polystyrene, fluoropolymer, PTFE, CYTOP, poly(methyl methacrylate) (PMMA), acrylic, epoxy and polydimethylsiloxane (PDMS).

G7. The method of any one of embodiments G1-G6, wherein an electrode is disposed on the substrate.

G8. The method of any one of embodiments G1-G7, wherein the well is disposed in the layer.

G9. The method of embodiment G8, wherein the well comprises a bottom and the electrode is disposed at the bottom of the well.

G10. The method of any one of embodiments G1-G9, wherein the electrode disposed at the well is a working electrode and the system comprises a reference electrode disposed at a position outside of a well.

G11. The method of embodiment G10, wherein the reference electrode is disposed on or in the chip.

G12. The method of any one of embodiments G1-G11, wherein each well is a microwell.

G13. The method of any one of embodiments G1-G12, wherein the chip comprises multiple wells and a working electrode is disposed at each well.

G14. The method of embodiment G13, wherein the system comprises a reference electrode disposed at a position outside of a well.

G15. The method of embodiment G14, wherein the reference electrode is disposed on or in the chip.

H1. The method of any one of embodiments A01-A19, B1-B34, C1-C11, D1-D5, E1-E6, F1-F45 and G1-G15, wherein the magnet is disposed on a moveable mount.

H2. The method of embodiment H1, wherein the moveable mount has a travel distance of about 1 mm to about 100 mm.

H3. The method of embodiment H1 or H2, wherein the magnet is disposed at a first position in parts (a) and (b) and is disposed at a second position in parts (c) and (d).

H4. The method of embodiment H3, wherein the magnet in the first position is further from the nanopore than in the second position.

H5. The method of embodiment H3 or H4, wherein the second position is about 100 micrometers to about 25 mm from the magnetic particle of the conjugate.

H6. The method of any one of embodiments H1-H5, wherein the moveable mount is a two-position mount.

H7. The method of any one of embodiments H1-H6, wherein the magnet comprises a permanent magnet.

H8. The method of embodiment H7, wherein the magnet comprises a ferromagnetic material.

H9. The method of embodiment H7, wherein the magnet comprises a rare earth magnetic material.

H10. The method of embodiment H7, wherein the rare earth magnetic material comprises a neodymium alloy.

H10.1. The method of any one of embodiments H1-H10, wherein the system comprises a Mu-metal strip disposed on or in a chip adjacent to a well.

H10.2. The method of any one of embodiments H1-H10.1, wherein system comprises a magnetic tip or a focusing yoke positioned adjacent to the magnet.

H10.3. The method of any one of embodiments H1-H10.2, wherein the magnet comprises magnet elements disposed in a side-by-side orientation.

H11. The method of any one of embodiments H1-H10.3, wherein the magnet comprises one magnet.

H12. The method of any one of embodiments H1-H10.3, wherein the magnet comprises two or more magnets each separated by a distance.

H13. The method of any one of embodiments H7-H12, wherein each magnet has a lateral width of about 0.1 mm to about 20 mm.

H14. The method of embodiment H12 or H13, wherein the distance is about 0.05 mm to about 20 mm.

H15. The method of any one of embodiments H1-H13, wherein the magnet comprises an electromagnet.

H16. The method of embodiment H15, wherein the magnet comprises one or more electromagnet elements.

H17. The method of embodiment H16, wherein the elements are in dipole or quadrupole arrangements.

H18. The method of any one of embodiments H1-H17, wherein the magnet is a magnetic tweezer.

I1. The method of any one of embodiments A01-A19, B1-B34, C1-C11, D1-D5, E1-E6, F1-F45, G1-G15 and H1-H18, wherein the magnetic particle of the conjugate comprises one or more nanoparticles.

I2. The method of embodiment I1, wherein each of the nanoparticles is defined by a diameter or effective diameter of about 1 nm to about 20 nm.

I3. The method of embodiment I1 or I2, wherein the magnetic particle comprises multiple nanoparticles in a matrix material.

I4. The method of embodiment I3, wherein the matrix material comprises one or more of gold, polystyrene and silica.

I5. The method of embodiment I3 or I4, wherein the magnetic particle has a diameter or effective diameter of about 200 nm to about 10 micrometers.

I6. The method of embodiment I5, wherein the magnetic particle has a diameter or effective diameter of about 1 micrometer to about 3 micrometers

I7. The method of any one of embodiments A01-A19, B1-B34, C1-C11, D1-D5, E1-E6, F1-F45, G1-G15, H1-H18 and I1-I6, wherein the magnetic particle comprises a superparamagnetic material.

I8. The method of embodiment I7, wherein the superparamagnetic material comprises a ferromagnetic material.

I9. The method of embodiment I8, wherein the ferromagnetic material comprises a ferromagnetic metal or ferromagnetic alloy.

I10. The method of embodiment I9, wherein the ferromagnetic metal or ferromagnetic alloy comprises one or more of iron, nickel, cobalt, platinum, manganese and iron oxide.

I11. The method of embodiment I10, wherein the magnetic particle comprises magnetite.

I12. The method of any one of embodiments A01-A19, B1-B34, C1-C11, D1-D5, E1-E6, F1-F45, G1-G15, H1-H18 and I1-I11, wherein the magnetic particle comprises one or more passivation polymers.

I13. The method of embodiment I12, wherein the passivation polymers are chosen from one or more of a polyethylene glycol, polyacrylate, polysaccharide and peptide.

I14. The method of any one of embodiments A01-A19, B1-B34, C1-C11, D1-D5, E1-E6, F1-F45, G1-G15, H1-H18 and I1-I13, wherein the magnetic particle is linked by covalent attachment to the polymer.

I15. The method of any one of embodiments A01-A19, B1-B34, C1-C11, D1-D5, E1-E6, F1-F45, G1-G15, H1-H18 and I1-I13, wherein the magnetic particle is linked by non-covalent attachment to the polymer.

I16. The method of embodiment I15, wherein:
the magnetic particle and the polymer are linked by linkage of an affinity tag to a complementary affinity tag,
the affinity tag is linked by covalent attachment to the magnetic particle, and
the complementary affinity tag is linked by covalent attachment to the polymer.

I16.1. The method of embodiment I16, wherein the affinity tag and complementary affinity tag are chosen independently from biotin/streptavidin, biotin/neutravidin, antigen/antibody, antigen/antibody fragment and polynucleotide/complimentary polynucleotide.

I17. The method of embodiment I16.1, wherein the polymer is linked to streptavidin and the magnetic particle is linked to biotin or the magnetic particle is linked to streptavidin and the polymer is linked to biotin.

I18. The method of any one of embodiments A01-A19, B1-B34, C1-C11, D1-D5, E1-E6, F1-F45, G1-G15, H1-H18 and I1-I17, wherein the magnetic particle of the conjugate does not contact the magnet in (a)-(d).

J1. The method of any one of embodiments A01-A19, B1-B34, C1-C11, D1-D5, E1-E6, F1-F45, G1-G15, H1-H18 and I1-I18, wherein the chip comprises one or more sensor sites and a nanopore at each sensor site.

J2. The method of embodiment J1, wherein the chip comprises 2 to about 1000 sensor sites.

J3. The method of embodiment J1 or J2, wherein:
each sensor site comprises a well, and
the well comprises an opening, a spanning membrane and a nanopore-containing protein disposed in the membrane.

J4. The method of embodiment J3, wherein the nanopore-containing protein is an ion channel protein, transmembrane protein, pore-forming protein or porin protein.

J5. The method of embodiment J4, wherein the nanopore-containing protein is a hemolysin protein.

J6. The method of embodiment J5, wherein the nanopore-containing protein is a bacterial alpha-hemolysin protein.

J7. The method of embodiment J4, wherein the nanopore-containing protein is a mycobacterial porin protein.

J8. The method of any one of embodiments J3-J7, wherein the nanopore-containing protein is a native protein, or contains one or more chemical modifications and/or one or more amino acid substitutions.

J9. The method of embodiment J8, wherein the nanopore-containing protein contains one or more amino acid substitutions within the nanopore of the protein.

J10. The method of embodiment J9, wherein the substitutions are to charged amino acids.

J11. The method of embodiment J10, wherein the substitutions are to positively charged amino acids.

J12. The method of any one of embodiments A01-A19, B1-B34, C1-C11, D1-D5, E1-E6, F1-F45, G1-G15, H1-H18, I1-I18 and J1-J11, wherein the chip contains no motor protein or enzyme.

K1. A nanopore polymer sequencing system, comprising:
a chip comprising a substrate, a layer disposed on the substrate, a sensor site disposed in the layer, an electrode disposed at the sensor site, an amplifier in connection with the electrode; and a nanopore disposed in the sensor site,
a magnet disposed in a cis position relative to the nanopore;
and
a circuit comprising the electrode, an amplifier in connection with the electrode, a data acquisition (DAQ) system in connection with the amplifier, a bias module in connection with the amplifier, and a control module in connection with the bias module and the DAQ system;
the circuit is configured to maintain a voltage bias level at the sensor site based on a state of the nanopore.

K1.1. The system of embodiment K1, wherein the state of the nanopore is chosen from one or more of: no polymer in association with the nanopore or a polymer translocation event.

K1.2. The system of embodiment K1.1, wherein the translocation event is a polymer capture event, polymer exit event or polymer reentry event.

K1.3. The system of any one of embodiments K1-K1.2, wherein the system comprises a well or chamber disposed in a trans position to the nanopore.

K1.4. The system of embodiment K1.3, wherein the well or chamber comprises a hard stop agent capable of forming a distal hard stop structure on a magnetic particle-polymer conjugate.

K1.5. The system of any one of embodiments K1.1-K1.4, wherein the translocation event is a distal hard stop and nanopore interaction event.

K1.6. The system of any one of embodiments K1-K1.4, comprising a sample flow channel disposed in a cis position relative to the nanopore and in a trans position relative to the magnet.

K1.7. The system of any one of embodiments K1-K1.6, wherein the circuit is configured to automatically control the voltage bias level at the sensor site in response to the state of the nanopore detected by the control module.

K1.8. The system of embodiment K1.7, wherein the circuit is configured to:
apply a first voltage bias level at the sensor site according to a first state of the nanopore is detected by the control module; and
apply a second voltage bias level at the sensor site according to a second state of the nanopore is detected by the control module.

K1.9. The system of embodiment K1.8, wherein:
the first state of the nanopore is different than the second state of the nanopore; and
the first voltage bias level and the second voltage bias level are the same or different.

K2. The system of any one of embodiments K1.3-K1.9, wherein the circuit is configured to iteratively apply a voltage bias at the sensor site, wherein each iteration comprises a voltage bias modification.

K3. The system of embodiment K2, wherein the circuit is configured to iteratively apply a voltage bias modification at the sensor site in response to a polymer translocation event detected by the control module.

K4. The system of embodiment K2 or K3, wherein the control module of the circuit comprises one or more of: a field-programmable gate array (FPGA), a microprocessor, memory, a microcontroller, a computer, an application specific integrated circuit (ASIC) and a fixed hardware circuit.

K5. The system of any one of embodiments K1-K4, wherein the DAQ system comprises: one or more digital-to-analog converters (DACs), one or more analog-to-digital converters (ADCs) and optionally the control module.

K6. The system of embodiment K5, wherein:
the ADC is configured to acquire an analog signal originating from the electrode disposed at the sensor site and transmit digitized current levels to the control module; and
the DAC is configured to receive instructions from the control module to set preset voltage bias levels for the bias module.

K7. The system of embodiment K6, wherein the control module is configured to:
transmit instructions to set preset voltage bias level selections;
transmit instructions to the bias module to select a first voltage bias level;
detect a translocation event from the digitized current levels; and
transmit instructions to the bias module to select a second voltage bias level based on the state of the nanopore.

K8. The system of any one of embodiments K1-K7, wherein the control module comprises memory and the translocation events occur at predetermined set points stored in the memory.

K9. The system of any one of embodiments K1-K8, wherein:
the electrode is in a trans position relative to the nanopore;
the sensor site comprises a well or chamber; and
the well or chamber comprises an opening, a spanning membrane and a nanopore-containing protein disposed in the membrane.

K10. The system of any one of embodiments K1-K9, wherein the magnet is in magnetic field proximity to the nanopore.

K11. The system of any one of embodiments K1-K10, wherein the magnet is disposed on a moveable mount that assumes multiple positions, wherein one of the positions is in magnetic field proximity to the nanopore.

K12. The system of any one of embodiments K1-K11, wherein the chip comprises a plurality of sensor sites.

K12.1. The system of embodiment K12, wherein the chip comprises about 1 to about 10,000 sensor sites.

K13. The system of any one of embodiments K1-K12.1, wherein the DAQ system comprises the control module.

K14. The system of any one of embodiments K1-K13, wherein the amplifier is a resistive feedback amplifier.

K15. The system of any one of embodiments K1-K13, wherein the amplifier is a capacitive feedback amplifier.

K16. The system of any one of embodiments K1-K15, wherein the circuit is an application-specific integrated circuit (ASIC).

K17. The system of embodiment K16, wherein the ASIC comprises the DAQ system and the control module.

K18. The system of any one of embodiments K1-K17, wherein the amplifier is an independent capacitive feedback amplifier.

K19. The system of any one of embodiments K1-K17, wherein the amplifier is an independent resistive feedback amplifier.

K20. The system of any one of embodiments K5-K15, wherein one or more of the DACs are configured to produce a voltage bias level that is applied to an electrode disposed at the sensor site.

K21. The system of any one of embodiments K5-K15, wherein one or more of the DACs are configured to produce preset voltage bias levels.

K22. The system of embodiment K21, wherein the voltage bias levels are produced with a noise of 20 nV/rtHz or less.

F23. The system of any one of embodiments K1-K22, wherein the DAQ system comprises an analog signal condition circuit.

K24. The system of embodiment K23, wherein the analog signal condition circuit comprises one or more of: a filter, gain stage and attenuator.

K25. The system of any one of embodiments K1-K24, comprising a reference electrode.

K25.1. The system of embodiment K25, wherein the reference electrode is in connection with the amplifier.

K25.2. The system of embodiment K25 or K25.1, wherein the reference electrode is an external reference electrode.

K26. The system of any one of embodiments K25-K25.2, wherein the electrode disposed at the sensor site is a working electrode disposed in a trans position relative to the nanopore and the reference electrode is disposed in a cis position relative to the nanopore.

K27. The system of embodiment K25 or K26, wherein the chip comprises multiple sensor sites and multiple nanopores, with a nanopore at each of the sensor sites, and a working electrode in proximity to each of the nanopores.

K28. The system of embodiment K27, wherein the circuit comprises a multiplexer circuit.

K29. The system of embodiment K28, wherein the multiplexer circuit is configured to select preset voltage bias levels produced by one or more of the DACs.

K30. The system of embodiment K29, wherein the preset voltage bias levels are selected by the multiplexer circuit manually or according to a routine managed by the control module.

K31. The system of any one of embodiments K27-K30, wherein the system comprises multiple amplifiers and each amplifier is paired with a dedicated multiplexer circuit.

K32. The system of any one of embodiments K5-K24, wherein the control module is in connection with one or more of the ADCs and one or more of the DACs.

K33. The system of embodiment K25, wherein the control module is configured to receive the current measurements from one of more of the ADCs.

K34. The system of embodiment K32 or K33, wherein the control module is configured to detect one or more of a polymer capture event, polymer exit event, polymer reentry event and distal hard stop and nanopore interaction event.

K35. The system of embodiment K34, wherein one or more of the polymer capture event, polymer exit event, polymer reentry event and distal hard stop and nanopore interaction event occur at predetermined set points stored in the control module.

K36. The system of embodiment K34 or K35, wherein the control module is configured to transmit to one or more of the DACs one or more of the capture voltage bias, holding voltage bias, exit voltage bias and reentry voltage bias levels.

K37. The system of any one of embodiments K34-K36, wherein the control module is configured to transmit to the multiplexer circuit a change in the voltage bias level to (i) the holding voltage bias level after detecting the polymer capture event, or (ii) the exit voltage bias level after detecting the polymer capture event, or (iii) the reentry voltage bias level after detecting the polymer exit event, or (iv) the exit voltage bias level after detecting the polymer reentry event, or (v) the reentry voltage bias level after detecting the distal hard stop and nanopore interaction event, or (vi) a distal hard stop stripping voltage bias after detecting the distal hard stop and nanopore interaction event, or a combination of two, three or some or all of (i), (ii), (iii), (iv), (v) and (vi), or a combination of (i) and (ii), (i) and (iii), (i) and (iv), (i) and (v), (i) and (vi), (ii) and (iii), (ii) and (iv), (ii) and (v), (ii) and (vi), (iii) and (iv), (iii) and (v), (iii) and (vi), (iv) and (v), (iv) and (vi) or (v) and (vi); or a combination of (i), (ii), (iii), (iv), (v); or (i), (ii), (iii), (iv), (vi), or (ii), (iii), (iv), (v) and (vi).

K38. The system of any one of embodiments K34-K37, wherein the control module is configured to transmit to one or more of the DACs a magnet activation signal after detecting the capture event.

K39. The system of any one of embodiments K1-K38, wherein the substrate comprises a material chosen from quartz, silica, glass, sapphire, printed circuit board laminate, polycarbonate and polytetrafluoroethylene (PTKE).

K40. The system of embodiment K39, wherein the substrate comprises fused quartz and/or fused silica.

K41. The system of any one of embodiments K1-K40, wherein the substrate comprises a thickness of about 0.1 mm to about 2.0 mm.

K42. The system of any one of embodiments K1-K41, wherein the layer comprises a thickness of about 0.1 micrometers to about 500 micrometers.

K43. The system of ay one of embodiments K1-K42, wherein the layer comprises one or more of epoxy-based negative photoresist, SU-8, polyimide, parylene, polystyrene, fluoropolymer, PTKE, CYTOP, poly(methyl methacrylate) (PMMA), acrylic, epoxy and polydimethylsiloxane (PDMS).

K44. The system of any one of embodiments K1-K43, wherein the electrode is disposed on the substrate.

K45. The system of any one of embodiments K1-K44, wherein the sensor site is a well disposed in the layer.

K46. The system of embodiment K45, wherein the well comprises a bottom and the electrode is disposed at the bottom of the well.

K47. The system of any one of embodiments K1-K46, wherein the electrode disposed at the well is a working electrode and the system comprises a reference electrode.

K47.1. The system of embodiment K47, wherein the reference electrode is in connection with the amplifier.

K47.2. The system of embodiment K47 or K47.1, wherein the reference electrode is an external reference electrode.

K47.3. the system of any one of embodiments K47-K47.2, wherein the reference electrode is disposed at a position outside of a well.

K48. The system of any one of embodiments K47-K47.3, wherein the reference electrode is disposed on or in the chip.

K49. The system of any one of embodiments K1-K48, wherein the chip comprises multiple wells and a working electrode disposed at each well.

K50. The system of embodiment K49, wherein each well is a microwell.

K51. The system of embodiment K49 or K50, wherein the system comprises a reference electrode disposed at a position outside of a well.

K51.1. The system of embodiment K51, wherein the reference electrode is in connection with the amplifier.

K51.2. The system of embodiment K51 or K51.1, wherein the reference electrode is an external reference electrode.

K52. The system of embodiment K51, wherein the reference electrode is disposed on or in the chip.

K53. The system of any one of embodiments K1-K52, wherein the magnet is disposed on a fixed mount or on a moveable mount.

K54. The system of embodiment K53, wherein the moveable mount has a travel distance of about 1 mm to about 100 mm.

K55. The system of embodiment K53 or K54, wherein the magnet is disposed at a first position in parts (a) and (b) and is disposed at a second position in parts (c) and (d).

K56. The system of embodiment K55, wherein the magnet in the first position is further from the nanopore than in the second position.

K57. The system of embodiment K55 or K56, wherein the second position is about 100 micrometers to about 10 mm from the nanopore.

K58. The system of any one of embodiments K53-K57, wherein the moveable mount is a two-position mount.

K59. The system of any one of embodiments K1-K58, wherein the magnet comprises a permanent magnet.

K60. The system of embodiment K59, wherein the magnet comprises a ferromagnetic material.

K61. The system of embodiment K59, wherein the magnet comprises a rare earth magnetic material.

K62. The system of embodiment K59, wherein the rare earth magnetic material comprises a neodymium alloy.

K63. The system of any one of embodiments K1-K62, wherein the magnet comprises one magnet.

K64. The system of any one of embodiments K1-K62, wherein the magnet comprises two or more magnets each separated by a distance.

K65. The system of embodiment K64, wherein each magnet has a lateral width of about 0.1 mm to about 20 mm.

K66. The system of embodiment K64 or K65, wherein the distance is about 0.05 mm to about 20 mm.

K67. The system of any one of embodiments K1-K66, wherein the magnet comprises an electromagnet.

K68. The system of embodiment K67, wherein the magnet comprises one or more electromagnet elements.

K69. The system of embodiment K68, wherein the elements are in dipole or quadrupole arrangements.

K70. The system of any one of embodiments K1-K69, wherein the magnet is a magnetic tweezer.

K70.1. The system of any one of embodiments K1-K70, wherein the system comprises a Mu-metal strip disposed on a chip adjacent to a well.

K70.2. The system of any one of embodiments K1-K70.1, wherein system comprises a magnetic tip or a focusing yoke positioned adjacent to the magnet.

K70.3. The system of any one of embodiments K1-K70.2, wherein the magnet comprises magnet elements disposed in a side-by-side orientation K71. The system of any one of embodiments K1-K70.3, comprising a cover between the sensor site and the magnet.

K72. The system of embodiment K71, wherein the cover is spaced from the sensor site.

K73. The system of embodiment K71 or K72, comprising a pump.

K74. The system of embodiment K73, wherein the pump is configured to transmit fluid between the sensor site and the cover.

K75. The system of any one of embodiments K1-K74, wherein the chip comprises 1 to about 1000 sensor sites.

K76. The system of any one of embodiments K1-K75, wherein:
each sensor site comprises a well, and
the well comprises an opening, a spanning membrane and a nanopore-containing protein disposed in the membrane.

K77. The system of embodiment K76, wherein the nanopore-containing protein is an ion channel protein, transmembrane protein, pore-forming protein or porin protein.

K78. The system of embodiment K77, wherein the nanopore-containing protein is a hemolysin protein.

K79. The system of embodiment K78, wherein the nanopore-containing protein is a bacterial alpha-hemolysin protein.

K80. The system of embodiment K77, wherein the nanopore-containing protein is a mycobacterial porin protein.

K81. The system of any one of embodiments K76-K80, wherein the nanopore-containing protein is a native protein, or contains one or more chemical modifications and/or one or more amino acid substitutions.

K82. The system of embodiment K81, wherein the nanopore-containing protein contains one or more amino acid substitutions within the nanopore of the protein.

K83. The system of embodiment K82, wherein the substitutions are to charged amino acids.

K84. The system of embodiment K83, wherein the substitutions are to positively charged amino acids.

K85. The system of any one of embodiments K1-K84, wherein the chip contains no motor protein or enzyme.

K86. The system of any one of embodiments K1-K85, in combination with a polymer-magnetic particle conjugate.

K87. The system of any one of embodiments K1-K86, the layer and/or membrane is passivated.

K89. The system of embodiment K87, wherein the membrane optionally is a bilayer membrane, lipid membrane, black lipid membrane, block copolymer membrane, diblock copolymer membrane, dual block copolymer membrane, and/or triblock copolymer membrane; optionally comprises one or more types of amphiphilic molecules; optionally comprises one or more types of polypeptides, optionally comprises one or more types of block polymers, diblock polymers, dual block polymers and/or triblock polymers; optionally comprises one or more types of lipids; and/or optionally is a planar lipid bilayer (PLB).

K90. The system of embodiment K89, wherein the membrane includes a headgroup region containing lipid headgroups.

K91. The system of embodiment K89 or K90, wherein lipids in the PLB contain a headgroup selected to reduce an attractive interaction between the PLB and the magnetic particle.

K92. The system of embodiment K91, wherein the PLB contains lipids containing a phosphatidyl serine headgroup.

K93. The system of embodiment K92, wherein the PLB contains one or more of phosphatidylcholine (PC), phosphatidylethanolamine (PE), phosphatidylglycerol (PG), phosphatidic acid (PA) and phosphatidyl serine (PS).

K94. The system of embodiment K89 or K90, wherein lipids in the PLB contain a headgroup associated with a passivating agent.

K95. The system of embodiment K94, wherein the passivating agent comprises PEG, PMOXA, hydroxyl, betaine, nitrile, carboxylate, methyl amide, amine, polypeptide, peptide, polysaccharide, polystyrene sulfonate, polysaccharide or a polyacrylate group.

K96. The system of embodiment K94, wherein the passivating agent is a surface-active passivating agent.

K97. The system of embodiment K96, wherein the surface-active passivating agent comprises a hydrophobic component that is capable associating with a hydrophobic region of the PLB, and a hydrophilic component that is capable of associating with remains the headgroup region.

K98. The system of embodiment K94 or K95, wherein the surface-active passivating agent is a surfactant.

K99. The system of embodiment K98, wherein the surfactant comprises sodium dodecyl sulfate (SDS), Tween-20, Triton X-100, octyl glucoside, digitonin, a phospholipid, a block copolymer, or a polymer covalently attached to a phospholipid or cholesterol.

K100. The system of any one of embodiments K90-K99, wherein the layer and/or membrane comprises a PEG headgroup modified phospholipid.

K101. The system of embodiment K100, wherein the PEG headgroup modified phospholipid is 1,2-dioleoyl-glycerophosphoethanolamine.

K102. The system of any one of embodiments K95-K101, wherein PEG has a molecular weight of about 0.1 kDa to about 100 kDa, or about 1 kDa to about 10 kDa, or about 5 kDa.

K103. The system of any one of embodiments K9-K102, wherein the membrane is a crosslinked membrane.

K104. The system of embodiment K103, wherein the crosslinked membrane optionally is a bilayer membrane, lipid membrane, black lipid membrane, block copolymer membrane, diblock copolymer membrane, dual block copolymer membrane, and/or triblock copolymer membrane; optionally comprises an amphiphilic molecule; optionally comprises a block polymer, diblock polymer, dual block polymer and/or triblock polymer; optionally comprises a lipid; optionally comprises a surfactant; and optionally comprises a polypeptide.

K105. The system of embodiment K104, wherein the amphiphilic molecule comprises one or more of a lipid, block copolymer, diblock copolymer, dual block copolymer, triblock copolymer, fatty acid and surfactant.

K106. The system of embodiment K105, wherein the lipid comprises a phospholipid.

K107. The system of any one of embodiments K103-K106, wherein the crosslinked membrane is a planar lipid bilayer (PLB).

K108. The system of any one of embodiments K103-K107, wherein the crosslinked membrane comprises covalent bonds linking adjacent amphiphilic molecules in the membrane.

K109. The system of any one of embodiments K103-K108, wherein the crosslinked membrane comprises polymerized reactive monomers.

K110. The system of embodiment K109, wherein the reactive monomers are embedded in the membrane and form extended polymers.

K111. The system of any one of embodiments K103-K110, wherein the crosslinked membrane, relative to a membrane that is not crosslinked, (i) increases the mechanical stability of the membrane, (ii) increase the voltage stability of the membrane, (iii) increases the lifetime of the membrane in the system, (iv) increases the stability of the nanopore within the membrane, (v) reduces nonspecific adsorption of a conjugate, or (vi) a combination of two or more of (i), (ii), (iii), (iv) and (v).

K112. The system of any one of embodiments K103-K111, wherein the crosslinked membrane comprises amphiphilic molecules containing headgroups.

K113. The system of any one of embodiments K103-K112, wherein the crosslinked membrane is crosslinked by applying a polymer having individual subunits that interact with amphiphilic molecules in the membrane.

K114. The system of embodiment K113, wherein headgroup moieties amphiphilic molecules interact with the polymer through intermolecular interactions or the formation of chemical bonds.

K115. The system of embodiment K113 or K114, wherein the polymer is chosen from poly L-lysine and actin.

K116. The system of any one of embodiments K103-K115, wherein the crosslinked membrane comprises chemically modified amphiphilic molecules containing a polymerizable reactive functional group.

K117. The system of embodiment K116, wherein the polymerizable reactive functional group is disposed within a hydrophobic region of the membrane, and links adjacent amphiphilic molecules in the hydrophobic region.

K118. The system of embodiment K117, wherein the polymerizable reactive functional group is chosen from an alkene or alkyne group.

K119. The system of embodiment K117 or K118, wherein the amphiphilic molecules are chosen from diacetylenic lipids and bis-dienoyl phosphatidylcholine (bis-DenPC) lipids.

K120. The system of any one of embodiments K117-K119, wherein the amphiphilic molecules comprise 1-palmitoyl-2,10,12-tricosadiynoyl-sn-glycero-3-phosphoethanolamine (PTPE).

K121. The system of any one of embodiments K 117-K120, wherein the polymerizable reactive functional group is disposed on headgroups of amphiphilic molecules, and links adjacent amphiphilic molecules.

K121. The system of embodiment K121, wherein the polymerizable reactive functional group is chosen from an alkyne, acrylate, methacrylate and alkene group.

K122. The system of any one of embodiments K103-K121, wherein the crosslinked membrane is crosslinked by dissolving hydrophobic reactive monomers in the membrane and initiating a chemical reaction to link the monomers together to form an extended polymer within the membrane.

K123. The system of embodiment K122, wherein the hydrophobic reactive monomers are dissolved in a hydrophobic region of the layer or membrane.

K124. The system of embodiment K122 or K123, wherein the hydrophobic reactive monomers are chosen from styrene, divinylbenzene, butyl methacrylate and ethylene glycol dimethacrylate.

K125. The system of any one of embodiments K116-K120, wherein the layer or membrane comprises 1,2-diphytanoyl-sn-glycero-3-phosphocholine (DPhPC) and PTPE diacetylenic lipid.

K126. The system of embodiment K125, wherein the DPhPC and PTPE diacetylenic lipid are in a ratio of about 1:1 by mass.

K127. The system of any one of embodiments K1-K126, wherein the chamber comprises a hard stop agent capable of associating with a polymer of a conjugate molecule when a portion of the polymer of the conjugate molecule is disposed in the chamber.

K128. The system of any one of embodiments K127, wherein the hard stop agent can be associated with the polymer by a covalent bond, or non-covalent interaction, or covalent bond, and non-covalent interaction.

K129. The system of embodiment K127 or K128, wherein the hard stop agent comprises an affinity tag.

K130. The system of embodiment K127 or K128, wherein the hard stop agent consists of an affinity tag.

K131. The system of embodiment K129 or K130, wherein the affinity tag has a hydrodynamic diameter larger than the diameter of the constriction region of the nanopore.

K132. The system of any one of embodiments K129-K131, wherein the affinity tag is chosen from streptavidin, neutravidin, antibody, antibody fragment, nucleic acid, DNA, RNA and artificial nucleic acid.

K133. The system of any one of embodiments K129-K131, comprising a conjugate containing a magnetic particle and a polymer, wherein and the polymer comprises a complementary affinity tag.

K134. The system of embodiment K133, wherein the complementary affinity tag of the polymer can enter and traverse the nanopore.

K135. The system of embodiment K133 or K134, wherein the complementary affinity tag of the polymer is chosen from biotin, antigen, and polynucleotide capable of hybridizing to complementary polynucleotide present in the hard stop agent.

K136. The system of any one of embodiments K127-K135, wherein:
the hard stop agent comprises a reactive group capable of reacting with a complementary reactive group disposed on a polymer contained in a conjugate; and
the reactive group and complementary reactive group are capable of forming a covalent bond.

K137. The system of embodiment K136, wherein the reactive group and complementary reactive group are chosen from amine/carboxylate, alkyne/azide, or thiol/maleimide.

K138. The system of any one of embodiments K127-K137, wherein the hard stop agent comprises a branched polymer, a nanoparticle, a protein, or a folded DNA structure.

K139. The system of embodiment K138, wherein the folded DNA structure is chosen from an i-motif, a G-quadruplex, a DNA duplex, a DNA-RNA duplex, a RNA-RNA duplex, and a DNA origami structure.

K140. The system of any one of embodiments K129-K139, wherein the hard stop agent comprises an affinity tag and a reactive group capable of reacting with a complementary reactive group disposed on a polymer contained in a conjugate.

K141. The system of embodiment K140, wherein the system comprises a polymer and the polymer comprises a complementary affinity tag.

K142. The system of any one of embodiments K127-K141, wherein the hard stop agent is a hard stop oligonucleotide comprising a polynucleotide complementary and capable of hybridizing to a polynucleotide of a polymer contained in a conjugate.

K143. The system of embodiment K141 or K142, wherein the oligonucleotide or polynucleotide of the capture molecule is about 10 to about 200 contiguous nucleotides in length.

K144. The system of embodiments K142 or K143, wherein the hard stop oligonucleotide, or polynucleotide complementary and capable of hybridizing to a polynucleotide of the polymer, comprises one or more locked nucleotides.

K145. The system of any one of embodiments K127-K144, wherein the hard stop agent is at a concentration in the compartment of about 100 μM to about 1 mM.

K146. The system of embodiment K145, wherein the hard stop agent is at a concentration in the compartment of about 100 nM to about 50 μM.

K147. The system of any one of embodiments A03.10-A03.26, wherein the hard stop agent comprises or consists of an affinity tag capable of associating with a complementary affinity tag disposed on a polymer contained in a conjugate, the affinity tag is streptavidin or neutravidin and the complementary affinity tag is biotin disposed on the distal terminus of the polymer.

K148. A conjugate or product of manufacture, comprising a polymer linked, or operatively linked, to a magnetic particle.

K149. The conjugate or product of manufacture of embodiment K148, wherein the polymer comprises a nucleic acid or polypeptide.

K150. The conjugate or product of manufacture of embodiment K149, wherein the polymer comprises a nucleic acid.

K151. The conjugate or product of manufacture of embodiment K150, wherein the nucleic acid is single stranded or double stranded.

K152. The conjugate or product of manufacture of embodiment K150 or K151, wherein the nucleic acid is DNA, RNA or contains one or more nucleotide analogs.

K153. The conjugate or product of manufacture of any one of embodiments K148-K152, wherein the magnetic particle of the conjugate comprises one or more nanoparticles.

K154. The conjugate or product of manufacture of embodiment K153, wherein each of the nanoparticles is defined by a diameter or effective diameter of about 1 nm to about 20 nm.

K155. The conjugate or product of manufacture of embodiment K153 or K154, wherein the magnetic particle comprises multiple nanoparticles in a matrix material.

K156. The conjugate or product of manufacture of embodiment K155, wherein the matrix material comprises one or more of gold, polystyrene and silica.

K157. The conjugate or product of manufacture of embodiment K155 or K156, wherein the magnetic particle has a diameter or effective diameter of about 200 nm to about 10 micrometers.

K158. The conjugate or product of manufacture of embodiment K157, wherein the magnetic particle has a diameter or effective diameter of about 1 micrometer to about 3 micrometers K159. The conjugate or product of manufacture of any one of embodiments K148-K158, wherein the magnetic particle comprises a superparamagnetic material.

K160. The conjugate or product of manufacture of embodiment K159, wherein the superparamagnetic material comprises a ferromagnetic material.

K161. The conjugate or product of manufacture of embodiment K160, wherein the ferromagnetic material comprises a ferromagnetic metal or ferromagnetic alloy.

K162. The conjugate or product of manufacture of embodiment K161, wherein the ferromagnetic metal or ferromagnetic alloy comprises one or more of iron, nickel, cobalt, platinum, manganese and iron oxide.

K163. The conjugate or product of manufacture of embodiment K162, wherein the magnetic particle comprises magnetite.

K164. The conjugate or product of manufacture of any one of embodiments K148-K163, wherein the magnetic particle is linked by covalent attachment to the polymer.

K165. The conjugate of any one of embodiments K1-K163, wherein the magnetic particle is linked by non-covalent attachment to the polymer.

K166. The conjugate or product of manufacture of embodiment K165, wherein:
the magnetic particle and the polymer are linked by linkage of an affinity tag to a complementary affinity tag,
the affinity tag is linked by covalent attachment to the magnetic particle, and
the complementary affinity tag is linked by covalent attachment to the polymer.

K167. The conjugate or product of manufacture of embodiment K166, wherein the affinity tag and complementary affinity tag are chosen independently from biotin/streptavidin, biotin/neutravidin, antigen/antibody, antigen/antibody fragment and polynucleotide/complimentary polynucleotide.

K168. The conjugate or product of manufacture of any one of embodiments K148-K167, wherein the magnetic particle is passivated.

K169. The conjugate or product of manufacture of embodiment K168, wherein the magnetic particle is associated with a passivating agent.

K170. The conjugate or product of manufacture of embodiment K169, wherein the passivating agent is a surface-active passivating agent.

K171. The conjugate or product of manufacture of embodiment K170, wherein the surface-active passivating agent adsorbs to the magnetic particle surface.

K172. The conjugate or product of manufacture of embodiment K170 or K171, wherein the surface-active passivating agent is not covalently attached to a magnetic particle.

K173. The conjugate or product of manufacture of embodiment K170 or K171, wherein the surface-active passivating agent is not associated with a magnetic particle via an affinity tag.

K174. The conjugate or product of manufacture of any one of embodiments K170-K173, wherein the surface-active passivating agent is a surfactant.

K175. The conjugate or product of manufacture of embodiment K174, wherein the surfactant is a detergent or polymer.

K176. The conjugate of embodiment K175, wherein the surfactant is chosen from sodium dodecyl sulfate (SDS), Tween-20, Triton X-100, octyl glucoside, digitonin, phospholipids, and pluronic block-copolymers.

K177. The conjugate or product of manufacture of any one of embodiments K170-K176, wherein the surface-active passivating agent is a polypeptide.

K178. The conjugate or product of manufacture of embodiment K177, wherein the polypeptide is an albumin.

K179. The conjugate or product of manufacture of embodiment K178, wherein the polypeptide is bovine serum albumin (BSA).

K180. The conjugate or product of manufacture of embodiment K169, wherein the passivating agent is attached to the magnetic particle by a covalent bond or by an affinity tag.

K181. The conjugate or product of manufacture of embodiment K180, wherein the passivating agent is ionic, zwitterionic, polar, or a polymeric group.

K182. The conjugate or product of manufacture of embodiment K180 or K181, wherein the passivating agent is chosen from a polyethylene glycol (PEG) polymer, poly(2-methyl-2-oxazoline) polymer (PMOXA), hydroxyl, betaine, nitrile, carboxylate, methyl amide, amine, polypeptide, peptide, polysaccharide, polystyrene sulfonate, and polyacrylate.

K183. The conjugate or product of manufacture of any one of embodiments K180-K182, wherein the passivating agent is attached to the magnetic particle by a covalent bond.

K184. The conjugate or product of manufacture of embodiment K183, wherein the covalent bond is formed between a reactive functional group attached to the passivating agent and a complementary functional group attached to the magnetic particle.

K185. The conjugate or product of manufacture of embodiment K184, wherein the reactive functional group and the complementary functional group are chosen from carboxyl/amine, NHS/amine, maleimide/thiol, thiol/thiol, amine/aldehyde, hydroxylamine/aldehyde, and alkyne/azide.

K186. The conjugate or product of manufacture of embodiment K185, wherein the reactive functional group and the complementary functional group is carboxyl/amine and the covalent bond is formed by reaction with a carbodiimide.

K187. The conjugate or product of manufacture of embodiment K186, wherein the carbodiimide is 1-ethyl-3-[3-dimethylaminopropyl]carbodiimide (EDC).

K188. The conjugate or product of manufacture of any one of embodiments K180-K187, wherein an affinity tag is attached to the passivating agent and a complementary affinity tag is attached to the magnetic particle.

K189. The conjugate or product of manufacture of embodiment K188, wherein the affinity tag and the complementary affinity tag are chosen independently from biotin/streptavidin, biotin/neutravidin, antigen/antibody, antigen/antibody fragment and polynucleotide/complimentary polynucleotide.

K190. The conjugate or product of manufacture of any one of embodiments K169-K189, wherein the passivating agent is a PEG polymer.

K191. The conjugate or product of manufacture of embodiment K190, wherein the PEG polymer has a molecular weight of about 0.1 kDa to about 100 kDa, or about 1 kDa to about 10 kDa, or about 5 kDa.

K192. The conjugate or product of manufacture of any one of embodiments K148-K191, wherein the polymer comprises an affinity tag, or a reactive group, or an affinity tag and a reactive group.

K193. The conjugate or product of manufacture of embodiment K193, wherein the affinity tag, or the reactive group or the affinity tag and the reactive group, are disposed in a distal portion or distal terminus of the polymer.

K194. The conjugate or product of manufacture of embodiment K193, wherein the distal portion of the polymer is disposed about 1 to about 1,000 contiguous nucleotides from the distal terminus of the polymer.

K195. The conjugate or product of manufacture of any one of embodiments K192-K194, wherein the affinity tag is capable of associating with a complementary affinity tag on a separate molecule.

K196. The conjugate or product of manufacture of embodiment K194, wherein the affinity tag and the complementary affinity tag are chosen independently from biotin/streptavidin, biotin/neutravidin, antigen/antibody, antigen/antibody fragment and polynucleotide/complimentary polynucleotide.

K197. The conjugate or product of manufacture of any one of embodiments K192-K196, wherein the reactive group is capable of associating with a complementary reactive group on a separate molecule.

K198. The conjugate or product of manufacture of embodiment K197, wherein the reactive group and complementary reactive group are capable of forming a covalent bond.

K199. The conjugate or product of manufacture of embodiment K198, wherein the reactive group and complementary reactive group are chosen from amine/carboxylate, alkyne/azide and thiol/maleimide.

K200. The conjugate or product of manufacture of any one of embodiments K195-K199, wherein the separate molecule is a hard stop agent.

K201. The system of any one of embodiments K1-K147 comprising a conjugate of any one of embodiments K148-K200.

K202. The system of embodiment K201, wherein the layer and/or membrane in the system and the magnetic particle of the conjugate each are passivated.

K203. The system of embodiment K202, wherein the layer and/or membrane in the system and the magnetic particle of the conjugate each are passivated with PEG.

K204. The system of embodiment K203, wherein the PEG independently has a molecular weight of about 0.1 kDa to about 100 kDa, or about 1 kDa to about 10 kDa, or about 5 kDa.

K205. A kit comprising a system of any one of embodiments K1-K147.

K206. The kit of embodiment K202, comprising a conjugate of any one of embodiments K148-K200.

K207. The kit of embodiment K206, wherein the layer and/or membrane in the system and the magnetic particle of the conjugate each are passivated.

K208. The kit of embodiment K207, wherein the layer and/or membrane in the system and the magnetic particle of the conjugate each are passivated with PEG.

K209. The kit of embodiment K208, wherein the PEG independently has a molecular weight of about 0.1 kDa to about 100 kDa, or about 1 kDa to about 10 kDa, or about 5 kDa.

L1. A nucleic acid, comprising a central nucleic acid flanked by a terminal proximal region and a terminal distal region, wherein:
the proximal region comprises a magnetic particle; and
the distal region comprises a terminal single stranded polynucleotide.

L2. The nucleic acid of embodiment L1, in combination with a system of any one of embodiments K1-K147.

L3. The nucleic acid of embodiment L1 or L2, wherein the terminal single stranded polynucleotide of the distal region comprises a polynucleotide having the structure (X)ₙ, wherein X is a nucleotide and n is about 10 to about 80, or between about 8 and 88, consecutive nucleotides.

L4. The nucleic acid of any one of embodiments L1-L3, wherein the terminal single stranded polynucleotide is linked in the distal region by a chemical reactive group.

L5. The nucleic acid of any one of embodiments L1-L3, wherein the terminal single stranded polynucleotide in the distal region is not linked to the central nucleic acid by a chemical reactive group.

L6. The nucleic acid of any one of embodiments L1-L5, wherein the magnetic particle in the proximal region is linked by a chemical reactive group.

L7. The nucleic acid of any one of embodiments L1-L6, wherein the magnetic particle in the proximal region is linked by a binding pair member.

L8. The nucleic acid of embodiment L7, wherein the binding pair comprises a biotin member and an avidin, streptavidin or neutravidin member.

L9. The nucleic acid of any one of embodiments L1-L8, wherein the central nucleic acid is single stranded.

L10. The nucleic acid of any one of embodiments L1-L8, wherein the central nucleic acid is partially single stranded nucleic acid and partially double stranded.

L11. The nucleic acid of embodiment L10, wherein:
the central nucleic acid comprises (i) a first strand, and (ii) a series of second strands complementary to subsequences of the first strand; and
each of the subsequences of the first strand is separated from an adjacent subsequence in the first strand by one or more nucleotides.

L12. The nucleic acid of any one of embodiments L1-L8, wherein the central nucleic acid is double stranded and comprises a first strand and complementary second strand.

L12.1. The nucleic acid of any one of embodiments L10-L12, wherein the magnetic particle and the terminal single stranded polynucleotide are linked to the same strand of the central nucleic acid.

L13. The nucleic acid of any one of embodiments L10-L12.1, wherein in the proximal region:
the magnetic particle is linked to the first strand of the central nucleic acid, and
the second strand of the central nucleic acid is linked to a terminal single stranded polynucleotide.

L14. The nucleic acid of embodiment L13, wherein the terminal single stranded polynucleotide linked to the second strand of the central nucleic acid comprises a polynucleotide having the structure (Z)ₙ, wherein Z is a nucleotide the same or different than X and n is about 10 to about 80 consecutive nucleotides.

L15. The nucleic acid of any one of embodiments L10-L12.1, wherein in the proximal region:
the magnetic particle is linked to the first strand of the central nucleic acid, and
the second strand of the central nucleic acid is linked to a terminal blocking moiety.

L16. The nucleic acid of any one of embodiments L10-L12.1, wherein in the proximal region the magnetic particle is linked to the first strand and to the second strand of the central nucleic acid.

L17. The nucleic acid of any one of embodiments L10-L16, wherein the magnetic particle is the terminal moiety of the proximal region.

L18. The nucleic acid of any one of embodiments L10-L17, wherein in the terminal region the terminal single stranded polynucleotide is linked to the first strand of the central nucleic acid.

L19. The nucleic acid of embodiment L18, wherein in the terminal region the second strand of the central nucleic acid is linked to a terminal blocking moiety.

L20. The nucleic acid of embodiment L18, wherein:
in the proximal region the second strand of the central nucleic acid is linked to a terminal blocking moiety; and
in the terminal region the second strand of the central nucleic acid is linked to a terminal blocking moiety.

L20.1. The nucleic acid of embodiment L20, wherein the blocking moiety is defined by a width of about 2 nm to about 100 nm.

L20.2. The nucleic acid of embodiment L20, wherein the blocking moiety is defined by a width of about 3 nm to about 20 nm.

L21. The nucleic acid of any one of embodiments L15, L19 and L20-L20.2, wherein the terminal blocking moiety of the proximal region or the distal region independently is a G-quadruplex polynucleotide, biotin/streptavidin complex, biotin/neutravidin complex, antigen/antibody complex, antigen/antibody fragment complex, polynucleotide/complimentary polynucleotide complex, dendrimer, polysaccharide, polyethylene glycol, gold nanoparticle, or polystyrene nanoparticle.

L21.1. The nucleic acid of embodiment L21, wherein the G-quadruplex polynucleotide comprises four groups of three or more repeated sequential guanine bases with 1 nucleotide to about 7 consecutive non-guanine bases between each of the four groups.

L21.2. The nucleic acid of embodiment L21 or L21.1, wherein the G-quadruplex polynucleotide comprises, in the 5′ to 3′ direction, GGGTTAGGGTTAGGGTTAGGG or GGGCGGGCGCGAGGG AGGG.

L22. The nucleic acid of any one of embodiments L10-L21.2, wherein the terminal single stranded polynucleotide is linked directly to the first strand and/or the second strand of the central nucleic acid.

L24. The nucleic acid of any one of embodiments L10-L22, wherein the magnetic particle is linked to the first strand of the central nucleic acid by a chemical reactive group.

L25. The nucleic acid of any one of embodiments L10-L23, wherein the terminal blocking moiety is linked to the second strand of the central nucleic acid by a binding pair member.

L26. The nucleic acid of any one of embodiments L10-L25, wherein the magnetic particle is linked to the first strand of the central nucleic acid by a chemical reactive group.

L27. The nucleic acid of any one of embodiments L10-L26, wherein the terminal blocking moiety is linked to the second strand of the central nucleic acid by a binding pair member.

L28. The nucleic acid of embodiment L26 or L27, wherein the binding pair comprises a biotin member and an avidin, streptavidin or neutravidin member.

L29. The nucleic acid of any one of embodiments L10-L21, wherein:
the terminal single stranded polynucleotide, the magnetic particle and/or the terminal blocking moiety independently are linked to an adapter nucleic acid in the proximal region; and/or
the terminal single stranded polynucleotide and/or the terminal blocking moiety independently are linked to an adapter nucleic acid in the distal region.

L29.1. The nucleic acid of embodiment L29, wherein the single stranded polynucleotide comprises a proximal terminal end, and the proximal terminal end of the single stranded polynucleotide is linked to the adapter nucleic acid in the proximal region.

L29.2. The nucleic acid of embodiment L29 or L29.1, wherein the terminal blocking moiety is a proximal terminal blocking moiety, and the proximal terminal blocking moiety is linked to the adapter nucleic acid in the proximal region.

L29.3. The nucleic acid of any one of embodiments L29-L29.2, wherein the single stranded polynucleotide comprises a distal terminal end, and the distal terminal end of the single stranded polynucleotide is linked to the adapter nucleic acid in the distal region.

L29.4. The nucleic acid of any one of embodiments L29-L29.3, wherein the terminal blocking moiety is a distal terminal blocking moiety, and the distal terminal blocking moiety is linked to the adapter nucleic acid in the distal region.

L29.5. The nucleic acid of any one of embodiments L29.1-L29.4, wherein the proximal terminal end of the single stranded polynucleotide is disposed at or near the magnetic particle, the proximal terminal blocking moiety is at or near the magnetic particle, the distal terminal end of the single stranded polynucleotide is disposed opposite of and optionally furthest from the magnetic particle, the distal terminal blocking moiety is opposite of and optionally furthest from the magnetic particle.

L30. The nucleic acid of any one of embodiments L29-L29.5, wherein the adapter nucleic acid comprises a first portion comprising non-complementary strands.

L31. The nucleic acid of embodiment L30, wherein:
the first portion of the adapter comprises a first strand and a second strand;
the first strand of the first portion of the adapter is linked to the first strand of the central nucleic acid; and
the second strand of the first portion of the adapter is linked to the second strand of the central nucleic acid.

L32. The nucleic acid of embodiment L30 or L31, wherein the first portion is linked directly to the central nucleic acid.

L33. The nucleic acid of embodiment L30 or L31, wherein the adapter nucleic acid comprises a second portion linked directly to the central nucleic acid comprising complementary strands.

L34. The nucleic acid of any one of embodiments L30, L31 and L33, wherein:
the second portion of the adapter comprises a first strand and a second strand;
the first strand of the second portion of the adapter is linked to the first strand of the central nucleic acid and to the first strand of the first portion of the adapter; and
the second strand of the second portion of the adapter is linked to the second strand of the central nucleic acid and to the second strand of the first portion of the adapter.

L35. The nucleic acid of any one of embodiments L30-L34, wherein the terminal single stranded polynucleotide, the magnetic particle and/or the terminal blocking moiety independently are linked to the first portion of the adapter.

L36. The nucleic acid of embodiment L35, wherein the first strand of the first portion of the adapter nucleic acid in the proximal region is linked to the magnetic particle.

L37. The nucleic acid of embodiment L35, wherein the first strand and the second strand of the first portion of the adapter nucleic acid in the proximal region are linked to the magnetic particle.

L38. The nucleic acid of any one of embodiments L35-L37, wherein the first strand of the first portion of the adapter nucleic acid in the distal region is linked to the terminal single stranded polynucleotide.

L39. The nucleic acid of any one of embodiments L35, L36 and L38, wherein the second strand of the first portion of the adapter nucleic acid in the proximal region is linked to the terminal single stranded polynucleotide.

L40. The nucleic acid of any one of embodiments L35-L39, wherein the second strand of the first portion of the adapter nucleic acid in the distal region is linked to the terminal single stranded polynucleotide.

L41. The nucleic acid of any one of embodiments L35, L36 and L38-L40, wherein the second strand of the first portion of the adapter nucleic acid in the proximal region is linked to the terminal single stranded polynucleotide.

L42. The nucleic acid of any one of embodiments L35-L41, wherein the magnetic particle is linked by a chemical reactive group.

L43. The nucleic acid of any one of embodiments L35-L41, wherein the magnetic particle is linked by a binding pair member.

L44. The nucleic acid of any one of embodiments L35-L43, wherein the terminal single stranded polynucleotide is linked by a direct nucleotide-to-nucleotide linkage.

L45. The nucleic acid of any one of embodiments L35-L44, wherein the terminal blocking moiety is linked by a chemical reactive group.

L46. The nucleic acid of any one of embodiments L35-L45, wherein the terminal blocking moiety is linked by a binding pair member.

L47. The nucleic acid of embodiment L43 or L46, wherein the binding pair comprises a biotin member and an avidin, streptavidin or neutravidin member.

L48. The nucleic acid of any one of embodiment L1-L47, wherein the central nucleic acid comprises or consists of a native polynucleotide.

L49. The nucleic acid of any one of embodiments L4-L48, wherein the magnetic particle, terminal single stranded nucleic acid and/or terminal blocking moiety linked by a chemical reactive group or a direct nucleotide-to-nucleotide linkage are linked by a covalent bond in the proximal region or the distal region.

L50. The nucleic acid of any one of embodiments L1-L49, wherein one magnetic particle is linked to one nucleic acid molecule.

L51. The nucleic acid of embodiment L50, wherein (i) one magnetic particle is linked to one strand of a double stranded nucleic acid molecule, (ii) one magnetic particle is linked to each strand of a double stranded nucleic acid molecule, or (iii) one magnetic particle is linked to a single-stranded nucleic acid molecule.

L52. The nucleic acid of embodiment L50 or L51, wherein for a majority of nucleic acid molecules in a composition one nucleic acid molecule is linked to one magnetic particle and multiple nucleic acid molecules are not linked to one magnetic particle.

L53. The nucleic acid of any one of embodiments L1-L52, wherein the magnetic particle is passivated.

L54. The nucleic acid of embodiment L53, wherein the magnetic particle is associated with a passivating agent.

L55. The nucleic acid of embodiment L54, wherein the passivating agent is a surface-active passivating agent.

L56. The nucleic acid of embodiment L55, wherein the surface-active passivating agent adsorbs to the magnetic particle surface.

L57. The nucleic acid of embodiment L55 or L56, wherein the surface-active passivating agent is not covalently attached to a magnetic particle.

L58. The nucleic acid of embodiment L55 or L56, wherein the surface-active passivating agent is not associated with a magnetic particle via an affinity tag.

L59. The nucleic acid of any one of embodiments L53-L58, wherein the surface-active passivating agent is a surfactant.

L60. The nucleic acid of embodiment L59, wherein the surfactant is a detergent or polymer.

L61. The nucleic acid of embodiment L60, wherein the surfactant is chosen from sodium dodecyl sulfate (SDS), Tween-20, Triton X-100, octyl glucoside, digitonin, phospholipids, and pluronic block-copolymers.

L62. The nucleic acid of any one of embodiments L55-L58, wherein the surface-active passivating agent is a polypeptide.

L63. The nucleic acid of embodiment L62, wherein the polypeptide is an albumin.

L64. The nucleic acid of embodiment L63, wherein the polypeptide is bovine serum albumin (BSA).

L65. The nucleic acid of embodiment L64, wherein the passivating agent is attached to the magnetic particle by a covalent bond or by an affinity tag.

L66. The nucleic acid of embodiment L65, wherein the passivating agent is ionic, zwitterionic, polar, or a polymeric group.

L67. The nucleic acid of embodiment L65 or L66, wherein the passivating agent is chosen from a polyethylene glycol (PEG) polymer, poly(2-methyl-2-oxazoline) polymer (PMOXA), hydroxyl, betaine, nitrile, carboxylate, methyl amide, amine, polypeptide, peptide, polysaccharide, polystyrene sulfonate, and polyacrylate.

L68. The nucleic acid of any one of embodiments L65-L67, wherein the passivating agent is attached to the magnetic particle by a covalent bond.

L69. The nucleic acid of embodiment L68, wherein the covalent bond is formed between a reactive functional group attached to the passivating agent and a complementary functional group attached to the magnetic particle.

L70. The nucleic acid of embodiment L69, wherein the reactive functional group and the complementary functional group are chosen from carboxyl/amine, NHS/amine, maleimide/thiol, thiol/thiol, amine/aldehyde, hydroxylamine/aldehyde, and alkyne/azide.

L71. The nucleic acid of embodiment L70, wherein the reactive functional group and the complementary functional group is carboxyl/amine and the covalent bond is formed by reaction with a carbodiimide.

L72. The nucleic acid of embodiment L71, wherein the carbodiimide is 1-ethyl-3-[3-dimethylaminopropyl]carbodiimide (EDC).

L73. The nucleic acid of any one of embodiments L65, wherein an affinity tag is attached to the passivating agent and a complementary affinity tag is attached to the magnetic particle.

L74. The nucleic acid of embodiment L73, wherein the affinity tag and the complementary affinity tag are chosen independently from biotin/streptavidin, biotin/neutravidin, antigen/antibody, antigen/antibody fragment and polynucleotide/complimentary polynucleotide.

L75. The nucleic acid of any one of embodiments L67-L74, wherein the passivating agent is a PEG polymer.

L76. The nucleic acid of embodiment L75, wherein the PEG polymer has a molecular weight of about 0.1 kDa to about 100 kDa, or about 1 kDa to about 10 kDa, or about 5 kDa.

L77. The nucleic acid of any one of embodiments L1-L76, comprising an affinity tag, or a reactive group, or an affinity tag and a reactive group.

L78. The nucleic acid of embodiment L77, wherein the affinity tag, or the reactive group or the affinity tag and the reactive group, are disposed in a distal portion or distal terminus of the nucleic acid.

L79. The method of embodiment L78, wherein the distal portion of the polymer is disposed about 1 to about 1,000 contiguous nucleotides from the distal terminus of the polymer.

L80. The nucleic acid of any one of embodiments L77-L79, wherein the affinity tag is capable of associating with a complementary affinity tag on a separate molecule.

L81. The nucleic acid of embodiment L80, wherein the affinity tag and the complementary affinity tag are chosen independently from biotin/streptavidin, biotin/neutravidin, antigen/antibody, antigen/antibody fragment and polynucleotide/complimentary polynucleotide.

L82. The nucleic acid of any one of embodiments L77-L81, wherein the reactive group is capable of associating with a complementary reactive group on a separate molecule.

L83. The nucleic acid of embodiment L82, wherein the reactive group and complementary reactive group are capable of forming a covalent bond.

L84. The nucleic acid of embodiment L83, wherein the reactive group and complementary reactive group are chosen from amine/carboxylate, alkyne/azide and thiol/maleimide.

L85. The nucleic acid of any one of embodiments L80-L84, wherein the separate molecule is a hard stop agent capable of forming a distal hard stop structure.

L86. The nucleic acid of any one of embodiments L1-L85, comprising a feature of a conjugate of any one of embodiments K148-K200.

L87. The nucleic acid of any one of embodiments L1-L86, in combination with a system of any one of embodiments K1-K147.

L88. The nucleic acid of embodiment L87, wherein the layer and/or membrane in the system and the magnetic particle of the conjugate each are passivated.

L89. The nucleic acid of embodiment L88, wherein the layer and/or membrane in the system and the magnetic particle of the conjugate each are passivated with PEG.

L90. The nucleic acid of embodiment L89, wherein the PEG independently has a molecular weight of about 0.1 kDa to about 100 kDa, or about 1 kDa to about 10 kDa, or about 5 kDa.

L91. A kit comprising a system of any one of embodiments K1-K147 and a nucleic acid of any one of embodiments L1-L90.

L92. The kit of embodiment L91, wherein the layer and/or membrane in the system and the magnetic particle of the conjugate each are passivated.

L93. The kit of embodiment L92, wherein the layer and/or membrane in the system and the magnetic particle of the conjugate each are passivated with PEG.

L94. The kit of embodiment L93, wherein the PEG independently has a molecular weight of about 0.1 kDa to about 100 kDa, or about 1 kDa to about 10 kDa, or about 5 kDa.

M1. A method for preparing a modified nucleic acid that includes a central nucleic acid region, a proximal region on one side of the central nucleic acid region and a distal region on the other side of the central nucleic acid region, the method comprising:
providing an input nucleic acid;
linking a magnetic particle to a proximal end of the input nucleic acid; and
linking a terminal single stranded polynucleotide to a distal end of the input nucleic acid, thereby generating a nucleic acid comprising (i) a central nucleic acid region comprising the input nucleic acid, (ii) a proximal region comprising the magnetic particle, and (iii) a distal region comprising the terminal single stranded polynucleotide.

M2. The method of embodiment M1, wherein the input nucleic acid is unprocessed sample nucleic acid.

M3. The method of embodiment M1, comprising processing the input nucleic acid from sample nucleic acid.

M4. The method of any one of embodiments M1-M3, wherein the input nucleic is double stranded or comprises a double-stranded portion.

M4.1. The method of embodiment M4, wherein the magnetic particle and the terminal single stranded polynucleotide are linked to one strand.

M5. The method of any one of embodiments M1-M4.1, wherein the input nucleic acid comprises DNA, RNA or combination thereof.

M6. The method of any one of embodiments M4-M5, wherein the input nucleic acid comprises a blunt end on one end of the input nucleic acid or on both ends of the input nucleic acid.

M7. The method of any one of embodiments M4-M5, wherein:
the proximal end of the input nucleic acid comprises a single-stranded overhang, or
the distal end of the input nucleic acid comprises a single-stranded overhang, or
the proximal end of the input nucleic acid comprises a single-stranded overhang and the distal end of the input nucleic acid comprises a single-stranded overhang.

M8. The method of embodiment M7, comprising filling in the single-stranded overhang of the proximal end, the distal end or the proximal end and the distal end, of the input nucleic acid, thereby generating a blunt end on the proximal end, the distal end or the proximal end and the distal end, of the input nucleic acid.

M9. The method of embodiment M8, comprising contacting the input nucleic acid with an enzyme comprising a polymerase activity and nucleotides under conditions in which the enzyme extends an end of a strand of the input nucleic acid not containing the single-stranded overhang with the nucleotides, thereby generating an extension of the strand not containing the single-stranded overhang that is complementary to the single-stranded overhang.

M9.1. The method of any one of embodiments M1-M9, comprising contacting sample nucleic acid with fragmentation conditions under which sample nucleic acid is fragmented.

M9.2. The method of embodiment M9.1, wherein the fragmentation conditions comprise one or more of ultrasonication, restriction enzyme fragmentation and fragmentase digestion.

M10. The method of any one of embodiments M1-M9.2, comprising contacting the input nucleic acid with a proximal adapter under conditions in which the proximal adapter joins to the proximal end of the input nucleic acid.

M11. The method of embodiment M10, wherein the proximal adapter comprises (i) a first binding partner member, (ii) the magnetic particle, (iii) a blocking moiety, (iv) a terminal single-stranded polynucleotide, or (v) a combination of two, three, or all of (i), (ii), (iii) and (iv).

M12. The method of embodiment M10 or M11, wherein the proximal adapter comprises:
a first strand and a second strand,
a distal region in which a portion of the first strand and a portion of the second strand are complementary, and
a proximal region in which a portion of the first strand and a portion of the second strand are not complementary; wherein the distal region of the proximal adapter joins to the proximal end of the input nucleic acid.

M13. The method of embodiment M12, wherein the first strand and the second strand of the proximal adapter each independently is about 10 to about 30 consecutive nucleotides in length.

M14. The method of embodiment M12 or M13, wherein the distal region of the proximal adapter comprises a blunt end or a single-stranded overhang.

M15. The method of any one of embodiments M12-M14, wherein the proximal region of the proximal adapter comprises (i) the first binding partner member, (ii) the magnetic particle, (iii) the blocking moiety, (iv) the terminal single-stranded polynucleotide, or (v) a combination of two, three, or all of (i), (ii), (iii) or (iv).

M16. The method of embodiment M15, wherein (i) the first binding partner member, (ii) the magnetic particle, (iii) the blocking moiety or (iv) the terminal single-stranded polynucleotide, is linked to the terminus of the first strand, the second strand, or the first strand and the second strand of the proximal adapter.

M17. The method of embodiment M16, wherein the first strand and the second strand of the proximal adapter are linked to the magnetic particle.

M18. The method of any one of embodiments M12-M17, comprising contacting the input nucleic acid with an enzyme under conditions in which the proximal adapter is joined to the input nucleic acid.

M19. The method of embodiment M18, wherein the enzyme comprises a ligase activity.

M20. The method of embodiment M10 or M11, wherein the proximal adapter comprises a nucleotide or nucleotide analog.

M21. The method of embodiment M20, comprising contacting the input nucleic acid with an enzyme capable of joining the proximal adapter to the proximal end of the input nucleic acid.

M22. The method of embodiments M21, wherein the enzyme joins the proximal adapter to a 5′ end of a strand of the input nucleic acid.

M23. The method of embodiment M22, wherein the enzyme comprises a kinase activity, ligase activity and/or capping enzyme complex activity.

M24. The method of embodiment M23, wherein the ligase activity is a T4 RNA ligase activity or Mth ligase activity.

M25. The method of embodiment M23, wherein the kinase activity is a T4 polynucleotide kinase activity.

M26. The method of embodiments M21, wherein the enzyme joins the proximal adapter to a 3′ end of a strand of the input nucleic acid.

M27. The method of embodiment M26, wherein the enzyme comprises a polymerase activity and/or a terminal deoxynucleotidyl transferase activity.

M28. The method of any one of embodiments M11-M27, wherein the proximal adapter comprises the first binding pair member.

M29. The method of embodiment M28, comprising contacting the proximal adapter with a component comprising a second binding pair member under conditions in which the first binding pair member joins to the second binding pair member.

M30. The method of embodiment M29, wherein the component comprising the second binding pair member is contacted with the proximal adapter before or after the proximal adapter is joined to the input nucleic acid.

M31. The method of any one of embodiments M11-M30, wherein the first binding pair member is linked to a glycosidic moiety or phosphoryl moiety of a nucleotide of the proximal adapter.

M32. The method of embodiment M31, wherein the first binding pair member is linked to a 2′ position of a glycosidic moiety or a gamma phosphate of the nucleotide.

M33. The method of any one of embodiment M29-M32, wherein the first binding pair member and the second binding pair member are joined by a covalent bond.

M34. The method of embodiment M33, wherein the first binding pair member or the second binding pair member is a chemically reactive group.

M35. The method of embodiment M34, wherein the first binding pair member or the second binding pair member independently comprises an azide, alkyne, amine, phosphorothioate or —SH moiety.

M36. The method of embodiment M35, wherein the first binding pair member or the second binding pair member independently comprises an iodoacetamide, dinitroflorobenzene, azide, or alkyne moiety.

M37. The method of any one of embodiments M29-M32, wherein the first binding pair member and the second binding pair member are joined by a non-covalent bond.

M38. The method of embodiment M37, wherein the first binding pair member and the second binding pair member are chosen independently from avidin, streptavidin, neutravidin and biotin.

M39. The method of any one of embodiments M1-M38, comprising contacting the input nucleic acid with a distal adapter under conditions in which the distal adapter joins to the distal end of the input nucleic acid.

M40. The method of embodiment M39, wherein the distal adapter comprises (i) a first binding partner member, (ii) a blocking moiety, (iii) a terminal single-stranded polynucleotide, or (iv) a combination of two or all of (i), (ii) and (iii).

M41. The method of embodiment M39 or M40, wherein the distal adapter comprises:
a first strand and a second strand,
a proximal region in which a portion of the first strand and a portion of the second strand are complementary, and
a distal region in which a portion of the first strand and a portion of the second strand are not complementary; wherein the proximal region of the distal adapter joins to the distal end of the input nucleic acid.

M42. The method of embodiment M41, wherein the first strand and the second strand of the distal adapter each independently is about 10 to about 30 consecutive nucleotides in length.

M43. The method of embodiment M40 or M41, wherein the distal region of the distal adapter comprises a blunt end or a single-stranded overhang.

M44. The method of any one of embodiments M41-M43, wherein the distal region of the distal adapter comprises (i) the first binding partner member, (ii) the blocking moiety, (iii) the terminal single-stranded polynucleotide, or (iv) combination of two or all of (i), (ii) and (iii).

M45. The method of embodiment M44, wherein (i) the first binding partner member, (ii) the blocking moiety or (iii) the terminal single-stranded polynucleotide, is linked to the terminus of the first strand, the second strand, or the first strand and the second strand of the distal adapter.

M46. The method of any one of embodiments M41-M45, comprising contacting the input nucleic acid with an enzyme under conditions in which the distal adapter is joined to the input nucleic acid.

M47. The method of embodiment M47, wherein the enzyme comprises a ligase activity.

M48. The method of embodiment M39 or M40, wherein the distal adapter comprises a nucleotide or nucleotide analog.

M49. The method of embodiment M48, comprising contacting the input nucleic acid with an enzyme capable of joining the distal adapter to a distal end of the input nucleic acid.

M50. The method of embodiments M49, wherein the enzyme joins the distal adapter to a 5′ end of a strand of the input nucleic acid.

M51. The method of embodiment M50, wherein the enzyme comprises a kinase activity, a ligase activity and/or a capping enzyme complex activity.

M52. The method of embodiment M51, wherein the ligase activity is a T4 RNA ligase activity or Mth ligase activity.

M53. The method of embodiment M51, wherein the kinase activity is a T4 polynucleotide kinase activity.

M54. The method of embodiments M49, wherein the enzyme joins the distal adapter to a 3′ end of a strand of the input nucleic acid.

M55. The method of embodiment M54, wherein the enzyme comprises a polymerase activity and/or a terminal deoxynucleotidyl transferase activity.

M56. The method of any one of embodiments M40-M55, wherein the distal adapter comprises the first binding pair member.

M57. The method of embodiment M56, comprising contacting the distal adapter with a component comprising a second binding pair member under conditions in which the first binding pair member joins to the second binding pair member.

M58. The method of embodiment M57, wherein the component comprising the second binding pair member is contacted with the distal adapter before or after the distal adapter is joined to the input nucleic acid.

M59. The method of any one of embodiments M40-M58, wherein the first binding pair member is linked to a glycosidic moiety or phosphoryl moiety of a nucleotide of the distal adapter.

M60. The method of embodiment M59, wherein the first binding pair member is linked to a 2′ position of a glycosidic moiety or a gamma phosphate of the nucleotide.

M61. The method of any one of embodiment M57-M60, wherein the first binding pair member and the second binding pair member are joined by a covalent bond.

M62. The method of embodiment M61, wherein the first binding pair member or the second binding pair member is a chemically reactive group.

M63. The method of embodiment M62, wherein the first binding pair member or the second binding pair member independently comprises an azide, alkyne, amine, phosphorothioate or —SH moiety.

M64. The method of embodiment M62 or M63, wherein the first binding pair member or the second binding pair member independently comprises an iodoacetamide, dinitroflorobenzene, azide, or alkyne moiety.

M65. The method of any one of embodiments M57-M60, wherein the first binding pair member and the second binding pair member are joined by a non-covalent bond.

M66. The method of embodiment M65, wherein the first binding pair member and the second binding pair member are chosen independently from avidin, streptavidin, neutravidin, and biotin.

M67. The method of any one of embodiments M1-M66, comprising contacting the input nucleic acid or modified input nucleic acid with an enzyme and nucleotides under conditions in which the enzyme generates and links a terminal single stranded polynucleotide comprising the nucleotides at the distal region, proximal region, or distal region and proximal region of the modified nucleic acid.

M68. The method of embodiment M67, wherein the enzyme comprises a polymerase activity.

M69. The method of any one of embodiments M1-M3, M5, M10, M11, M20-M40, and M48-M68, wherein the input nucleic acid is single-stranded.

M70. The method of embodiment M69, wherein the input nucleic acid comprises RNA.

M71. The method of embodiment M69 or M70, comprising joining the proximal adapter to the 5′ end of the input nucleic acid.

M72. The method of any one of embodiments M69-M71, comprising joining the distal adapter to the 3′ end of the input nucleic acid.

M73. The method of any one of embodiments M69-M71, comprising contacting the input nucleic acid or modified input nucleic acid with an enzyme and nucleotides under conditions in which the enzyme generates and links a terminal single stranded polynucleotide comprising the nucleotides at the 3′ end of the input nucleic acid.

M74. The method of any one of embodiments M1-M68, comprising contacting single-stranded sample nucleic acid with a plurality of oligonucleotides under conditions in which the oligonucleotides hybridize to the sample nucleic acid, thereby generating input nucleic acid comprising the single-stranded sample nucleic acid and a plurality of the oligonucleotides hybridized to the single-stranded sample nucleic acid.

M75. The method of embodiment M74, wherein:
there is a gap between the ends of two adjacent oligonucleotides in the input nucleic acid; and
the gap is at least one nucleotide or two or more consecutive nucleotides in length.

M76. The method of any one of embodiments M1-M68, comprising contacting single-stranded sample nucleic acid with a primer under conditions in which the primer hybridizes to the single-stranded sample nucleic acid.

M77. The method of embodiment M76, comprising contacting the sample nucleic acid with nucleotides and an enzyme comprising a reverse transcriptase activity and/or polymerase activity under conditions in which the enzyme extends the primer and generates a double-stranded input nucleic acid or an input nucleic acid comprising a double-stranded region.

M78. The method of any one of embodiments M1-M77, comprising separating from sample nucleic acid an internal region, wherein the internal region separated from the sample nucleic acid is the input nucleic acid.

M79. The method of embodiment M78, comprising contacting the sample nucleic acid with a gene-editing polypeptide and/or gene-editing enzyme under conditions in which the polypeptide and/or enzyme separates the internal region from the sample nucleic acid.

M80. The method of embodiment M79, wherein the gene-editing polypeptide and/or gene-editing enzyme is chosen from a CRISPR (clustered regularly interspaced short palindromic repeats) associated (Cas) polypeptide, CRISPR-associated endonuclease in Prevotella and Francisella 1 (Cpf1), transcription activator-like effector (TALE) protein, transcription activator-like effector nuclease (TALENs), zinc finger protein, endonuclease, integrase, recombinase, transposase, restriction enzyme and hybrid protein.

M81. The method of embodiment M80, wherein the Cas polypeptide is chosen from a Cas3 polypeptide, Cas9 polypeptide and Cas10 polypeptide.

M82. The method of embodiment M80, wherein the hybrid protein is a hybrid between a Cas9 polypeptide fused to a Clo51 endonuclease (Cas-CLOVER).

M83. The method of any one of embodiments M79-M83, comprising contacting the sample nucleic acid with a guide RNA.

M84. The method of embodiment M78, comprising contacting the sample nucleic acid with an endonuclease.

M85. The method of any one of embodiments M78-M84, comprising modifying an end of the sample nucleic acid with dideoxynucleotide.

M86. The method of embodiment M85, comprising contacting the sample nucleic acid with a dideoxynucleotide and an enzyme comprising a polymerase activity under conditions in which the enzyme appends the dideoxynucleotide to an end of the sample nucleic acid.

M87. The method of embodiment M85 or M86, wherein 3′ ends of double stranded sample nucleic acid are modified with a dideoxynucleotide.

M88. The method of any one of embodiments M85-M87, wherein the sample nucleic acid is modified with a dideoxynucleotide prior to separating the internal region from the sample nucleic acid.

M89. The method of any one of embodiments M78-M88, comprising hybridizing a first oligonucleotide and a second oligonucleotide to a single strand of input nucleic acid, wherein:
the first oligonucleotide comprises a distal region that is complementary to a region of the input nucleic acid and an adjacent proximal region not complementary to a region of the input nucleic acid;
the second oligonucleotide comprises a proximal region that is complementary to a region of the input nucleic acid and an adjacent distal region not complementary to a region of the input nucleic acid; and
the distal terminus of the region of the input nucleic acid complementary to the first oligonucleotide is separated by at least ten consecutive nucleotides from the proximal terminus of the region of the input nucleic acid complementary to the second oligonucleotide.

M90. The method of embodiment M89, comprising:
subjecting double-stranded input nucleic acid to denaturation conditions in which the strands of the input nucleic acid dissociate, thereby generating dissociated input nucleic acid strands; and
subjecting the dissociated input nucleic acid strands to annealing conditions in the presence of the first oligonucleotide and the second oligonucleotide, thereby generating a complex in which a strand of the input nucleic acid is hybridized to the first oligonucleotide and the second oligonucleotide.

M91. The method of embodiment M89 or M90, comprising joining the proximal end of the input nucleic acid to the proximal end of the first oligonucleotide and joining the distal end of the input nucleic acid to the distal end of the second oligonucleotide, thereby generating nucleic acid comprising circularly ligated ends.

M92. The method of embodiment M91, comprising contacting the complex with an enzyme comprising a ligase activity under conditions in which the proximal end of the input nucleic acid in the complex is joined to the proximal end of the first oligonucleotide and the distal end of the input nucleic acid in the complex is joined to the distal end of the second oligonucleotide.

M93. The method of embodiment M91 or M92, comprising subjecting the dissociated input nucleic acid strands to single-stranded nucleic acid degradation conditions.

M94. The method of embodiment M93, comprising contacting the dissociated input nucleic acid strands, after hybridizing the first oligonucleotide and the second oligonucleotide, to an enzyme comprising an exonuclease activity under conditions in which the enzyme cleaves dissociated input nucleic strands to which the first oligonucleotide and the second oligonucleotide have not hybridized.

M95. The method of embodiment M93 or M94, comprising joining the first oligonucleotide to the second oligonucleotide by an intervening fill oligonucleotide.

M96. The method of embodiment M95, comprising contacting the input nucleic acid hybridized to the first oligonucleotide and the second oligonucleotide with nucleotides, an enzyme comprising polymerase activity and an enzyme comprising a ligase activity under conditions in which the distal terminus of the first oligonucleotide is joined to the proximal terminus of the second oligonucleotide by a fill oligonucleotide complementary to the input nucleic acid.

M97. The method of any one of embodiments M91-M96, comprising cleaving the circularly ligated ends.

M98. The method of embodiment M97, wherein each of the circularly ligated ends is cleaved at a single position.

M99. The method of embodiment M98, wherein the first oligonucleotide and the second oligonucleotide comprise uracil.

M100. The method of embodiment M99, comprising cleaving the circularly ligated ends at the uracil.

M101. The method of embodiment M100, comprising contacting the circularly ligated ends with an enzyme comprising glycosylase activity under conditions in which the enzyme removes the uracil, thereby cleaving each of the circularly ligated ends is cleaved at a single position.

M102. The method of embodiment M101, wherein the enzyme comprises a uracil-DNA glycosylase (UDG) activity.

M103. The method of any one of embodiments M100-M102, wherein the proximal region of the first oligonucleotide comprises a first region and a second region separated by a uracil and the distal region of the second oligonucleotide comprises a first region and a second region separated by a uracil.

M104. The method of embodiment M103, wherein the first region of the first oligonucleotide, the second region of the first oligonucleotide, the first region of the second oligonucleotide and the second region of the second oligonucleotide independently form one of the following after cleaving at the uracil: a terminal single-stranded polynucleotide, blocking moiety and first binding pair member.

M105. The method of embodiment M104, wherein the first binding pair member is chosen from biotin, avidin, streptavidin, or neutravidin.

M106. The method of embodiment M104 or M105, wherein the first region of the first oligonucleotide forms a terminal single-stranded polynucleotide and the second region of the first oligonucleotide forms a blocking moiety after cleaving at the uracil.

M107. The method of any one of embodiments M104-M106, wherein the first region of the second oligonucleotide comprises the first binding pair member and the second region of the first oligonucleotide forms a blocking moiety after cleaving at the uracil.

M108. The method of embodiment any one of embodiments M104-M107, comprising contacting nucleic acid after cleaving at the uracil with an agent comprising the second binding pair member linked to a magnetic particle under conditions in which the first binding pair member and the second binding pair member associate.

M109. A nucleic acid, obtainable by a method of any one of embodiments M1-M108.

M110. A nucleic acid of any one of embodiments L1-L52, obtainable by a method of any one of embodiments M1-M108.

N1. A polymer, comprising a central polypeptide flanked by a terminal proximal region and a terminal distal region, wherein:
the proximal region comprises a magnetic particle; and
the distal region comprises a terminal polypeptide or a terminal single stranded polynucleotide.

N2. The polymer of embodiment N1, wherein:
the distal region comprises a terminal polypeptide;
the terminal polypeptide has the structure (Xₐₐ)ₙ, wherein Xₐₐ is an amino acid and n is about 10 to about 4,000 consecutive amino acids; and
Xₐₐ optionally is a charged amino acid.

N2.1. The polymer of embodiment N2, wherein the terminal polypeptide is about 300 to about 600 consecutive amino acids.

N2.2. The polymer of embodiment N2, wherein the terminal polypeptide is about 400 to about 500 consecutive amino acids.

N3. The polymer of any one of embodiments N1-N2.2, wherein the terminal polypeptide is a single alpha-helical polypeptide.

N4. The polymer of any one of embodiments N1-N3, wherein the proximal region, or the distal region, or the proximal region and the distal region, comprises a binding pair.

N5. The polymer of embodiment N4, wherein the binding pair comprises a covalently attached chemical reactive group member or a non-covalently attached binding pair member.

N6. The polymer of embodiment N5, wherein the non-covalently attached binding pair member comprises biotin, avidin, streptavidin or neutravidin.

N7. The polymer of embodiment N1, wherein:
the distal region comprises a terminal single stranded polynucleotide; and
the terminal single stranded polynucleotide comprises a polynucleotide having the structure (X)ₙ, wherein X is a nucleotide and n is about 10 to about 80 consecutive nucleotides.

N8. The polymer of any one of embodiments N1-N7, wherein the central polypeptide comprises or consists of a native polypeptide.

O1. A method for preparing a modified polymer that includes a central polypeptide region, a proximal region on one side of the central polypeptide region and a distal region on the other side of the central polypeptide region, the method comprising:
providing an input polypeptide;
linking a magnetic particle to a proximal end of the input polypeptide; and
linking a terminal single stranded polynucleotide or terminal polypeptide to a distal end of the input polypeptide, thereby generating a modified polymer comprising (i) a central polypeptide region comprising the input polypeptide, (ii) a proximal region comprising the magnetic particle, and (iii) a distal region comprising the terminal single stranded polynucleotide or terminal polypeptide.

O2. The method of embodiment O1, comprising contacting the input polypeptide with a first component comprising a first chemically reactive moiety and a magnetic particle and/or binding pair member under conditions in which the first chemically reactive moiety reacts with the proximal end of the input polypeptide.

O3. The method of embodiment O1 or O2, comprising contacting the input polypeptide with a second component comprising a second chemically reactive moiety and (i) a terminal single stranded polynucleotide, or (ii) terminal polypeptide or (iii) binding member pair, under conditions in which the second chemically reactive moiety reacts with the distal end of the input polypeptide.

O4. The method of any one of embodiments O1-O3, wherein:
the proximal end of the input polypeptide is a N-terminus and the distal end of the input polypeptide is a C-terminus, or
the proximal end of the input polypeptide is a C-terminus and the distal end of the input polypeptide is a N-terminus.

O5. The method of any one of embodiments O1-O4, wherein:
the terminal polypeptide of the distal region has the structure (Xₐₐ)ₙ, wherein Xₐₐ is an amino acid and n is about 10 to about 4,000 consecutive amino acids; and
Xₐₐ optionally is a charged amino acid.

O6. The method of embodiment O5, wherein the terminal polypeptide comprises or is a single alpha-helical polypeptide.

O7. The method of any one of embodiments O1-O6, wherein the proximal region, or the distal region, or the proximal region and the distal region, comprises a binding pair member.

O8. The method of embodiment O7, wherein the binding pair comprises a covalently attached chemical reactive group member or a non-covalently attached binding pair member.

O9. The method of embodiment O8, wherein the non-covalently attached binding pair member comprises biotin, avidin, streptavidin or neutravidin.

O10. The method of any one of embodiments O7-O9, comprising contacting the modified polypeptide with a modifying component comprising a binding pair member and (i) a magnetic particle, (ii) a terminal single stranded polynucleotide, (iii) a terminal polypeptide, (iv) a blocking moiety, or a combination of two or three of (i), (ii), (iii) and (iv), under conditions in which the binding pair member of the modifying component associates with and/or reacts with the binding pair member of the proximal region, the distal region or the proximal region and the distal region of the modified polypeptide.

O11. The method of any one of embodiments O2-O10, wherein the first component, or the second component, or the first component and the second component, independently comprises an adapter nucleic acid comprising a central double stranded complementary region, a distal double stranded or single stranded non-complementary region and a proximal double stranded or single stranded non-complementary region.

O12. The method of embodiment O11, wherein the first component, or the second component, or the first component and the second component, independently comprises (i) a chemically reactive moiety, a magnetic particle or binding pair member, and optionally a blocking moiety, or (ii) a chemically reactive moiety, a binding pair member or terminal single stranded polynucleotide, and optionally a blocking moiety.

O13. The method of any one of embodiments O2-O12, wherein the first component, or the second component, or the first component and the second component, independently comprises a single alpha-helical polypeptide.

O14. The method of any one of embodiments O6-O13, wherein the single alpha-helical polypeptide comprises about 10 to about 400 consecutive amino acids.

O15. The method of any one of embodiments O13 or O14, wherein the first component, or the second component, or the first component and the second component, independently comprises (i) a chemically reactive moiety and a magnetic particle or binding pair member, or (ii) a chemically reactive moiety, a binding pair member or terminal single stranded polypeptide.

O16. The method of any one of embodiments O1-O15, wherein the chemically reactive moiety is capable for forming a covalent bond with the N-terminus or C-terminus of the input polypeptide.

O17. The method of any one of embodiments O1-O16, wherein the input polypeptide comprises or consists of a native polypeptide.

O18. The method of any one of embodiments O2-O17, wherein the first chemically reactive moiety and the second chemically reactive moiety independently are chosen from maleimide, azide, NHS-ester, carboxy, and peptide enzymatic ligation tag moieties.

O19. The method of embodiment O18, comprising contacting a chemically reactive moiety comprising a peptide enzymatic ligation tag moiety with an enzyme comprising carboxypeptidase Y, sortase, or peptide asparaginyl ligase activity under conditions in which the enzyme links the ligation tag moiety to the input polypeptide.

O20. A modified polypeptide, obtainable by a method of any one of embodiments O1-O19.

O21. A polymer of any one of embodiments N1-N8, obtainable by a method of any one of embodiments O1-O19.

P1. A kit, comprising: a first adapter nucleic acid and a second adapter nucleic acid, wherein:
the first adapter nucleic acid comprises a first strand and a second strand, a complementary region in which a portion of the first strand and a portion of the second strand are complementary and a non-complementary region in which a portion of the first strand and a portion of the second strand are not complementary;
a terminus of the first strand of the first adapter nucleic acid in the non-complementary region is (i) linked to a magnetic particle, or (ii) linked to a first binding pair member to which a second binding pair member interacts;
the second adapter nucleic acid comprises a first strand and a second strand, a complementary region in which a portion of the first strand and a portion of the second strand are complementary and a non-complementary region in which a portion of the first strand and a portion of the second strand are not complementary;
a terminus of the first strand of the second adapter nucleic acid in the non-complementary region is (i) linked to a terminal single stranded nucleic acid, or (ii) linked to a first binding pair member to which a second binding pair member interacts.

P2. The kit of embodiment P1, wherein:
a terminus of the second strand of the first adapter nucleic acid in the non-complementary region optionally is linked to a blocking moiety; or
a terminus of the second strand of the second adapter nucleic acid in the non-complementary region optionally is linked to a blocking moiety; or
a terminus of the second strand of the first adapter nucleic acid in the non-complementary region optionally is linked to a blocking moiety and a terminus of the second strand of the second adapter nucleic acid in the non-complementary region optionally is linked to a blocking moiety.

P3. The kit of embodiment P1 or P2, wherein:
a terminus of the first strand of the first adapter nucleic acid in the non-complementary region is linked to a first binding pair member to which a second binding pair member interacts; and
the kit comprises a composition comprising a magnetic particle linked to the second binding pair member.

P4. The kit of embodiment P1 or P2, wherein a magnetic particle is linked to (i) a terminus of the first strand of the first adapter nucleic acid in the non-complementary region, and (ii) a terminus of the first strand of the first adapter nucleic acid in the non-complementary region.

P5. The kit of any one of embodiments P1-P4, wherein the first strand and the second strand covering the complementary region and the non-complementary region each independently are about 10 to about 30 consecutive nucleotides in length.

P6. A kit, comprising:
a first component comprising a first binding pair member;
a second component comprising an enzyme capable of linking the first component to a 5′ end of a nucleic acid;
a third component comprising a second binding pair member linked to a magnetic particle;
a fourth component comprising an enzyme capable of extending a 3′ end of a nucleic acid and nucleotides; and
optionally, a fifth component comprising a second binding pair member linked to a blocking moiety.

P7. The kit of embodiment P6, wherein:
(i) the first component comprises the first binding pair member linked to a ligase substrate or polynucleotide kinase substrate; or
(ii) the enzyme in the second component comprises a ligase activity, polynucleotide kinase activity and/or capping enzyme complex activity; or
(iii) the enzyme in the fourth component comprises a terminal deoxynucleotidyl transferase activity; or
(iv) the first binding pair member and the second binding pair member form a covalent bond via a chemical reaction or bind via a non-covalent interaction; or
(v) a combination of two or more or all of (i), (ii), (iii) and (iv).

P8. The kit of embodiment P7, wherein:
the first binding pair member and the second binding pair member form a covalent bond via a chemical reaction;
the first binding pair member or the second binding pair member independently comprises an azide, alkyne, amine, phosphorothioate or —SH moiety; and
the first binding pair member or the second binding pair member independently comprises an iodoacetamide, dinitroflorobenzene, azide, or alkyne moiety.

P9. A kit, comprising:
a first component comprising a first binding pair member;
a second component comprising an enzyme capable of linking the first component to a 5′ end of a nucleic acid;
a third component comprising a second binding pair member linked to a magnetic particle; and
optionally a fourth component comprising an enzyme capable of extending a 3′ end of a nucleic acid and nucleotides; or
optionally a fifth component comprising a component comprising a first binding pair member capable of being linked to the 3′ end of a nucleic acid and another component comprising a second binding pair member linked to a terminal single stranded nucleic acid.

P10. The kit of embodiment P9, wherein:
(i) the first component comprises the first binding pair member linked to a ligase substrate or polynucleotide kinase substrate; or
(ii) the enzyme in the second component comprises a ligase activity, polynucleotide kinase activity and/or capping enzyme complex activity; or
(iii) the enzyme in the fourth component comprises a terminal deoxynucleotidyl transferase activity capable of linking the nucleotides as a polynucleotide to the 3′ end of a nucleic acid and/or comprises a polymerase activity; or
(iv) the fifth component comprises an enzyme comprising a polymerase activity capable of linking the component comprising the first binding pair member to the 3′ end of a nucleic acid; or
(v) the first binding pair member and the second binding pair member form a covalent bond via a chemical reaction or bind via a non-covalent interaction; or
(vi) a combination of two or more or all of (i), (ii), (iii), (iv) and (v).

P11. The kit of embodiment P10, wherein:
the first binding pair member and the second binding pair member form a covalent bond via a chemical reaction;
wherein the first binding pair member or the second binding pair member independently comprises an azide, alkyne, amine, phosphorothioate or —SH moiety; and
the first binding pair member or the second binding pair member independently comprises an iodoacetamide, dinitroflorobenzene, azide, or alkyne moiety.

P12. A kit, comprising:
a first component comprising a first binding pair member;
a second component comprising an enzyme capable of linking the first component to a 3′ end of a nucleic acid;
a third component comprising a second binding pair member linked to a magnetic particle; and
a fourth component comprising an enzyme that cleaves a target region from a nucleic acid.

P13. The kit of embodiment P12, comprising a fifth component comprising a second binding pair member linked to a blocking moiety.

P14. The kit of embodiment P12 or P13, comprising:
a sixth component comprising a first binding pair member;
a seventh component comprising an enzyme capable of linking the sixth component to a 5′ end of a nucleic acid; and
an eighth component comprising a second binding pair member and a polynucleotide having the structure (X)ₙ, wherein X is a nucleotide and n is about 10 to about 80 consecutive nucleotides.

P15. The kit of any one of embodiments P12-P14, wherein:
(i) the sixth component comprises the first binding pair member linked to a ligase substrate or polynucleotide kinase substrate; or
(ii) the enzyme in the seventh component comprises a ligase activity, polynucleotide kinase activity and/or capping enzyme complex activity; or
(iii) the enzyme in the second component comprises a terminal deoxynucleotidyl transferase activity; or
(iv) the first binding pair member and the second binding pair member in the first and the third component form a covalent bond via a chemical reaction or bind via a non-covalent interaction; or
(v) a combination of two or more or all of (i), (ii), (iii) and (iv).

P16. The kit of embodiment P15, wherein:
the first binding pair member and the second binding pair member in the sixth component and the eighth component form a covalent bond via a chemical reaction;
the first binding pair member or the second binding pair member independently comprises an azide, alkyne, amine, phosphorothioate or —SH moiety; and
the second binding pair member independently comprises an iodoacetamide, dinitroflorobenzene, azide, or alkyne moiety.

P17. A kit, comprising:
a first component comprising an enzyme that cleaves a target region from a nucleic acid; and
a second component comprising:
   a first oligonucleotide comprising a first region complementary to a first subsequence of the target nucleic acid and a second region not complementary to a subsequence of the target nucleic acid; and
   a second oligonucleotide comprising a first region complementary to a second subsequence of the target nucleic acid different than the first subsequence and a second region not complementary to a subsequence of the target nucleic acid.

P18. The kit of embodiment P17, wherein the second region of the first oligonucleotide comprises a blocking moiety polynucleotide, a polynucleotide comprising deoxyuridine moieties and a polynucleotide having the structure (X)ₙ, wherein X is a nucleotide and n is about 10 to about 80 consecutive nucleotides.

P19. The kit of embodiment P17 or P18, wherein the second region of the second oligonucleotide comprises a blocking moiety polynucleotide, a polynucleotide comprising deoxyuridine moieties and a polynucleotide comprising a first binding pair member.

P20. The kit of any one of embodiments P17-P19, comprising a third component comprising one or more of (i) an enzyme comprising a ligase activity, (ii) an enzyme comprising an exonuclease activity, (iii) an enzyme comprising a polymerase activity, and (iv) an enzyme comprising a uracil-DNA glycosylase activity.

P21. The kit of embodiments P19 or P20, comprising a fourth component comprising a second binding pair member and a magnetic particle.

P22. The kit of embodiment P21, wherein the first binding pair member and the second binding pair member in the second and the fourth component form a covalent bond via a chemical reaction or bind via a non-covalent interaction.

P23. The kit of embodiment P22, wherein:
the first binding pair member and the second binding pair member in the second component and the fourth component form a covalent bond via a chemical reaction;
the first binding pair member or the second binding pair member independently comprises an azide, alkyne, amine, phosphorothioate or —SH moiety; and
the first binding pair member or the second binding pair member independently comprises an iodoacetamide, dinitroflorobenzene, azide, or alkyne moiety.

P24. The kit of any one of embodiments P1-P23, comprising a component comprising a plurality of oligonucleotide species each capable of hybridizing to a different subsequence of a single-stranded RNA, wherein each different subsequence is separated from an adjacent subsequence in RNA by one or more nucleotides.

P25. The kit of any one of embodiments P1-P23, comprising one or more primers capable of hybridizing to a single stranded RNA, nucleotides, and an enzyme comprising a reverse transcriptase activity and/or a polymerase activity capable of incorporating the nucleotides onto the primers using the RNA as a template.

P26. A kit, comprising:
a first component comprising a first chemically reactive moiety and a magnetic particle; and
a second component comprising a second chemically reactive moiety and a terminal polypeptide or a terminal single stranded polynucleotide; wherein:
the first chemically reactive moiety is capable of reacting with an N-terminus of a polypeptide and the second chemically reactive moiety is capable of reacting with a C-terminus of a polypeptide; or
the first chemically reactive moiety is capable of reacting with a C-terminus of a polypeptide and the second chemically reactive moiety is capable of reacting with an N-terminus of a polypeptide.

P27. The kit of embodiment P26, wherein:
the second component comprises a terminal polypeptide;
the terminal polypeptide has the structure (Xₐₐ)ₙ, wherein Xₐₐ is an amino acid and n is about 10 to about 4,000 consecutive amino acids; and
Xₐₐ optionally is a charged amino acid.

P27.1. The kit of embodiment P27, wherein the terminal polypeptide is about 300 to about 600 consecutive amino acids.

P27.2. The kit of embodiment P27, wherein the terminal polypeptide is about 400 to about 500 consecutive amino acids.

P28. The kit of any one of embodiments P26-P27.2, wherein the terminal polypeptide comprises or is a single alpha-helical polypeptide.

P29. The kit of embodiment P26, wherein:
the second component comprises a terminal single stranded polynucleotide; and
the terminal single stranded polynucleotide comprises a polynucleotide having the structure (X)ₙ, wherein X is a nucleotide and n is about 10 to about 80 consecutive nucleotides.

P30. The kit of any one of embodiments P26-P29, wherein:
the first chemically reactive moiety is capable of forming a covalent bond with the N-terminus of a polypeptide and the second chemically reactive moiety is capable of forming a covalent bond with a C-terminus of a polypeptide; or
the first chemically reactive moiety is capable of forming a covalent bond with a C-terminus of a polypeptide and the second chemically reactive moiety is capable of forming a covalent bond with an N-terminus of a polypeptide.

P31. The kit of embodiment P30, wherein the kit comprises a third component comprising a reagent that modifies the N-terminus of the polypeptide for reaction with the first chemically reactive moiety or the second chemically reactive moiety.

P32. The kit of embodiment P31, wherein the third component comprises a functionalized aldehyde or an azidoacetic anhydride.

P33. The kit of any one of embodiments P30-P32, wherein the kit comprises a third component comprising a reagent that modifies the C-terminus of the polypeptide for reaction with the first chemically reactive moiety or the second chemically reactive moiety.

P34. The kit of embodiment P33, wherein the third component comprises a carboxypeptidase Y activity or an ethylene-modified reactive group.

P35. The kit of any one of embodiments P26-P34, wherein the first chemically reactive moiety and the second chemically reactive moiety independently are chosen from maleimide, azide, NHS-ester, carboxy, and peptide enzymatic ligation tag moieties, and optionally comprising an enzyme comprising carboxypeptidase Y, sortase, or peptide asparaginyl ligase activity.

Q1. The method of any one of embodiments A1-A19, B1-B34, C1-C11, D1-D5, E1-E6, F1-F45, G1-G15, H1-H18, I1-I18, J1-J12, M1-M108 and O1-O19, wherein the terminal single stranded polynucleotide independently comprises a polynucleotide having the structure (X)ₙ, wherein X is a nucleotide and n is about 10 to about 80 consecutive nucleotides.

Q2. The method of any one of embodiments A1-A19, B1-B34, C1-C11, D1-D5, E1-E6, F1-F45, G1-G15, H1-H18, I1-I18, J1-J12, M1-M108, 01-019 and Q1, comprising a terminal magnetic particle.

Q3. The method of any one of embodiments A1-A19, B1-B34, C1-C11, D1-D5, E1-E6, F1-F45, G1-G15, H1-H18, J1-J12, M1-M108, 01-019, Q1 and Q2, comprising a magnetic particle including a feature of any one of embodiments I1-I18.

Q4. The method of any one of embodiments A1-A19, B1-B34, C1-C11, D1-D5, E1-E6, F1-F45, G1-G15, H1-H18, I1-I18, J1-J12, M1-M108, 01-019 and Q1-Q3, comprising a terminal blocking moiety.

Q5. The method of any one of embodiments A1-A19, B1-B34, C1-C11, D1-D5, E1-E6, F1-F45, G1-G15, H1-H18, I1-I18, J1-J12, M1-M108, 01-019 and Q1-Q4, wherein the blocking moiety independently is a G-quadruplex polynucleotide, biotin/streptavidin complex, biotin/neutravidin complex, antigen/antibody complex, antigen/antibody fragment complex, polynucleotide/complimentary polynucleotide complex, dendrimer, polysaccharide, polyethylene glycol, gold nanoparticle, or polystyrene nanoparticle.

Any of the above aspects and embodiments can be combined with any other aspect or embodiment as disclosed here in the Summary, Figures and/or Detailed Description sections.

As used in this specification and the claims, the singular forms “a,” “an” and “the” include plural referents unless the context clearly dictates otherwise.

Unless specifically stated or obvious from context, as used herein, the term “or” is understood to be inclusive and covers both “or” and “and”.

Unless specifically stated or obvious from context, as used herein, the term “about” is understood as within a range of normal tolerance in the art, for example within 2 standard deviations of the mean. About (use of the term “about”) can be understood as within 20%, 19%, 18%, 17%, 16%, 15%, 14%, 13%, 12% 11%, 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Unless specifically stated or obvious from context, as used herein, the terms “substantially all”, “substantially most of”, “substantially all of” or “majority of” encompass at least about 75%, 80%, 85%, 90%, 91%, 92%, 93%, 94%, 95%, 96%, 97%, 98%, 99% or 99.5%, or more of a referenced amount of a composition.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Incorporation by reference of these documents, standing alone, should not be construed as an assertion or admission that any portion of the contents of any document is considered to be essential material for satisfying any national or regional statutory disclosure requirement for patent applications. Notwithstanding, the right is reserved for relying upon any of such documents, where appropriate, for providing material deemed essential to the claimed subject matter by an examining authority or court.

Modifications may be made to the foregoing without departing from the basic aspects of the invention. Although the invention has been described in substantial detail with reference to one or more specific embodiments, those of ordinary skill in the art will recognize that changes may be made to the embodiments specifically disclosed in this application, and yet these modifications and improvements are within the scope and spirit of the invention. The invention illustratively described herein suitably may be practiced in the absence of any element(s) not specifically disclosed herein. Thus, for example, in each instance herein any of the terms “comprising”, “consisting essentially of”, and “consisting of” may be replaced with either of the other two terms. Thus, the terms and expressions which have been employed are used as terms of description and not of limitation, equivalents of the features shown and described, or portions thereof, are not excluded, and it is recognized that various modifications are possible within the scope of the invention. Embodiments of the invention are set forth in the following claims.

The invention will be further described with reference to the examples described herein; however, it is to be understood that the invention is not limited to such examples.

### EXAMPLES

The examples set forth below illustrate certain implementations and do not limit the technology.

### Example 1: Capturing a ssDNA Polymer Tethered to a Magnetic Particle and Generating a Pseudorotaxane Containing a Distal Hard Stop by Forming a DNA Duplex in the Trans Chamber

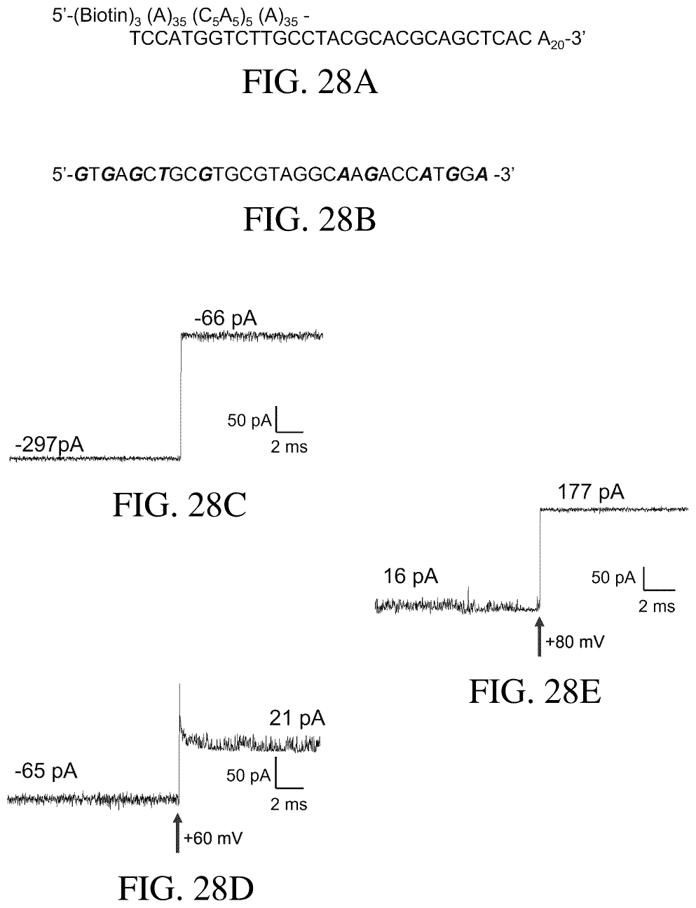

This example describes a target DNA capture event, and a demonstration of the formation of a pseudorotaxane, also referred to as a “hard stop,” detected by switching the applied voltage from negative to positive bias (cis vs. trans), followed by applying a high positive bias able to dissociate the DNA rotaxane and cause the target DNA to translocate out of the nanopore. The target DNA is referred to as a “DNA polymer.” The representative DNA polymer analyzed was a 170 nt single-stranded DNA with 3 biotin modifiers added sequentially to the 5′ end, an A35 homopolymer tail on the 3′ end, and a hybridization sub-sequence complementary to an oligonucleotide that would hybridize to the target polymer and form a hard stop on the trans side of the nanopore (FIG. 28A). The central portion of the DNA polymer contained a series of (A₅C₅) homopolymer repeats designed to produce a series of current steps (up-downs-up-downs) as this segment traverses the nanopore. The DNA polymer was bound at the proximal end to streptavidin-coated 2.8 μm magnetic polystyrene magnetic particles (Thermo Scientific M-270 Streptavidin).

The DNA polymer was immobilized by incubating a 1.7 μM solution of biotinylated DNA with particles for 20 minutes, followed by washing the particles to remove unbound DNA, thereby preparing modified target DNA. The reader was an M2-MspA protein nanopore supported in a DPhPC PLB formed over a glass nanopore membrane (GNM), in supporting electrolyte with 1 M KCl and 10 mM Tris buffered at pH 7.0. The interior trans chamber of the GNM was prepared with a 50 μM solution of an oligonucleotide that was 30 consecutive nucleotides in length, and is referred to as a “hard stop oligonucleotide” herein. The hard stop oligonucleotide contained a mix of deoxyribose and locked nucleic acid (LNA) nucleotides, having the structure illustrated in FIG. 28B. The sequence of the hard stop oligonucleotide was complementary to the hybridization sub-sequence within the target DNA polymer, and was designed to hybridize to the sub-sequence in the DNA polymer in the trans chamber after the modified target DNA was captured by the nanopore reader. When hybridized to the hybridization sub-sequence in the DNA polymer, the hard stop oligonucleotide was designed to contribute to a pseudorotaxane structure serving as a hard stop to prevent the DNA polymer from escaping the nanopore in the trans-to-cis direction.

To capture a DNA polymer modified by a magnetic particle, a 2 μL aliquot containing modified DNA polymers was injected into the test cell near the GNM surface, while applying a −160 mV potential between the cis and trans sides of the nanopore reader, and waiting up to 5 minutes for a modified DNA polymer to collide with the nanopore and the DNA portion to be captured. DNA capture events were identified by a rapid change in current from an open channel current of approximately −300 pA to a blocked current level of −66 pA, shown in FIG. 28C. After capturing a modified DNA, the strand was held for 30 seconds to allow hybridization with the complementary hard stop oligonucleotide in the trans chamber of the cell to form the hard stop structure. After formation of the rotaxane, the voltage bias was changed from −160 mV to +60 mV, producing an electrophoretic force that drove the DNA strand in the trans-to-cis direction. The duplex formed on the trans side of the nanopore with the LNA oligonucleotide served as a hard stop that prevented the target DNA from translocating in the trans-to-cis direction and exiting the nanopore, resulting in a long-lived current blockade, shown in FIG. 28D. At positive biases (cis-vs.-trans) higher than +60 mV, the electrophoretic force produced by the nanopore reader was high enough to induce dissociation of the LNA oligonucleotide and caused the DNA polymer to disassociate from the magnetic particle and rapidly translocate in a trans-to-cis direction out of the nanopore, resulting in an open-channel current. An example of this pseudorotaxane or hard stop “breaking” process is shown in FIG. 28E.

### Example 2: Multipassing a ssDNA Polymer with a Hard Stop and Tethered to a Magnetic Particle

This example describes a device and process in which a target DNA polymer (sequence shown in FIG. 28A) was attached to a magnetic particle captured by a protein nanopore reader, with a hard stop formed on the trans side, and passed back and forth through the polymer multiple times. The nanopore reader was an M2-MspA protein nanopore supported in a DPhPC PLB formed over a glass nanopore electrode, formed in supporting electrolyte with 1 M KCl and 10 mM Tris buffered at pH 7.0. The interior trans chamber of the GNM was prepared with a 50 μM solution of a hard stop oligonucleotide 30 consecutive nucleotides in length, and containing a mix of deoxyribose and locked nucleic acid (LNA) nucleotides, shown in FIG. 28B. The hard stop oligonucleotide hybridized to the target DNA polymer after being captured by the nanopore reader, contributing to a “pseudorotaxane” structure that served as a hard stop to prevent the DNA from escaping the nanopore in the trans-to-cis direction, as described in Example 1.

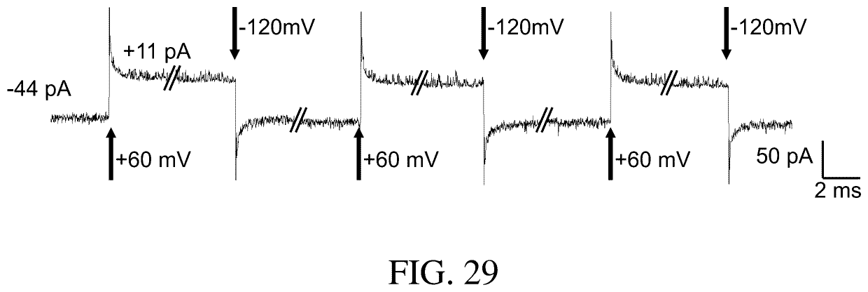

To capture target DNA modified by a magnetic particle (see description in Example 1), a 2 μL aliquot of modified target DNA was injected into the test cell near the GNM surface, while applying a −160 mV potential between the cis and trans sides of the nanopore reader, and waiting up to 5 minutes for a particle to collide with the nanopore and the DNA to be captured. DNA capture events were identified by a rapid change decrease in current from an open channel current of approximately 300 pA to a blocked current level of 65 pA. After capturing modified target DNA, the strand was held for 30 seconds to allow hybridization to the complementary hard stop oligonucleotide to form the hard stop structure. The DNA strand of the modified target DNA was then passed back and forth through the nanopore by switching the voltage from +60 mV (driving the DNA trans-to-cis) and −120 mV (cis-to-trans). Current traces of this multipassing process are shown in FIG. 29, in which the voltage transitions are indicated by a brief capacitive charging current spike that settles to the steady-state current level.

### Example 3: Capturing a ssDNA Tethered to a Magnetic Particle and Forming a Streptavidin-Based Hard Stop in the Trans Chamber

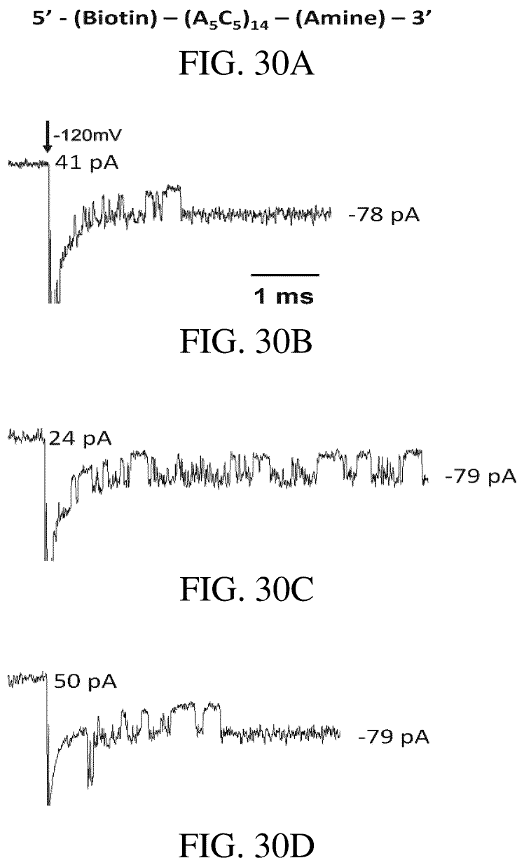

This example describes a single-stranded DNA polymer attached to a magnetic particle captured within a protein nanopore, with a streptavidin-biotin hard stop formed on the distal end, and passed back and forth through the nanopore reader by switching from a positive to negative voltage bias. The sample was prepared by a process similar to the processes described in Examples 1 and 2, except that a biotin-streptavidin interaction was used to form the hard stop within the trans chamber. The target ssDNA (FIG. 30A) was covalently attached to carboxylated 2.8 μm polystyrene magnetic particles (Thermo Scientific M-270 Carboxylic acid) through a primary amine modifier on the 3′ end of the DNA, thereby preparing modified target DNA. The 5′ end of the target DNA was modified with a terminal biotin group, which was able to bind with streptavidin present within the trans chamber of the GNM. The target DNA contained a repeating ASCS pattern. After injecting a 2 μL aliquot of target DNA modified by magnetic particles, a capture event was identified by a rapid current step down to deeper blocking state from an open channel current of approximately 300 pA at −160 mV recorded for an MspA nanopore reader. After capturing DNA tethered to a particle, the modified target DNA was held for 30 seconds to allow rotaxane formation, i.e., association of the streptavidin in the trans chamber to biotin linked to the captured modified target DNA. The applied voltage bias was then switched from −120 to +120 to drive the DNA in the trans-to-cis direction up to the hard stop, before stepping back to −120 mV to translocate the DNA in the cis-to-trans direction. Current traces for the voltage step from +120 mV to −120 mV are shown in FIGS. 30B-30C. These traces show current fluctuations upon recapture that are indicative of the A and C nucleobases traversing the sensing zone of the nanopore reader.

### REFERENCES

1. Sarkar, R.; Rybenkov, V. V., A Guide to Magnetic Tweezers and Their Applications. Frontiers in Physics 2016, 4.
2. Lipfert, J.; Hao, X.; Dekker, N. H., Quantitative Modeling and Optimization of Magnetic Tweezers. Biophysical Journal 2009, 96 (12), 5040-5049.
3. Ge, J.; Hu, Y.; Biasini, M.; Beyermann, W. P.; Yin, Y., Superparamagnetic Magnetite Colloidal Nanocrystal Clusters. Angewandte Chemie International Edition 2007, 46 (23), 4342-4345.
4. Lim, J.; Lanni, C.; Evarts, E. R.; Lanni, F.; Tilton, R. D.; Majetich, S. A., Magnetophoresis of Nanoparticles. ACS Nano 2011, 5 (1), 217-226.
5. nanoComposix: Hypertext Transfer Protocol Secure (https) address nanocomposix.com/pages/magnetite-nanoparticles.
6. Sievers, S.; et al, Quantitative measurement of the magnetic moment of individual magnetic nanoparticles by magnetic force microscopy. Small (Weinheim an der Bergstrasse, Germany) 2012, 8 (17), 2675-2679.
7. Stoddart, D.; et al., Single-nucleotide discrimination in immobilized DNA oligonucleotides with a biological nanopore. Proceedings of the National Academy of Sciences 2009, 106 (19), 7702-7707.
8. Laszlo, A. H.; Derrington, I. M.; Gundlach, J. H., MspA nanopore as a single-molecule tool: From sequencing to SPRNT. Methods 2016, 105, 75-89.
9. Nivala, J.; Marks, D. B.; Akeson, M., Unfoldase-mediated protein translocation through an [alpha]-hemolysin nanopore. Nat Biotech 2013, 31 (3), 247-250.
10. Nivala, J.; et al, Discrimination among Protein Variants Using an Unfoldase-Coupled Nanopore. ACS nano 2014, 8 (12), 12365-12375.
11. Strick, T. R.; Allemand, J.-F.; Bensimon, D.; Bensimon, A.; Croquette, V., The Elasticity of a Single Supercoiled DNA Molecule. Science 1996, 271 (5257), 1835-1837.
12. Zhao, X.; et al., Studying the mechanical responses of proteins using magnetic tweezers. Nanotechnology 2017, 28 (41), 414002.
13. Latinwo, F.; Schroeder, C. M., Model systems for single molecule polymer dynamics. Soft Matter 2011, 7 (18), 7907-7913.
14. Jagannathan, B.; Marqusee, S., Protein folding and unfolding under force. Biopolymers 2013, 99 (11), 860-869.
15. Essevaz-Roulet, B.; Bockelmann, U.; Heslot, F., Mechanical separation of the complementary strands of DNA. Proceedings of the National Academy of Sciences 1997, 94 (22), 11935-11940.
16. Lynch, S.; Baker, H.; Byker, S. G.; Zhou, D.; Sinniah, K., Single Molecule Force Spectroscopy on G-Quadruplex DNA. Chemistry—A European Journal 2009, 15 (33), 8113-8116.
17. de Messieres, M.; Chang, J.-C.; Brawn-Cinani, B.; La Porta, A., Single-Molecule Study of $G$-Quadruplex Disruption Using Dynamic Force Spectroscopy. Physical Review Letters 2012, 109 (5), 058101.
18. Bustamante, C.; Smith, S. B.; Liphardt, J.; Smith, D., Single-molecule studies of DNA mechanics. Current Opinion in Structural Biology 2000, 10 (3), 279-285.
19. Guo, S.; Ray, C.; Kirkpatrick, A.; Lad, N.; Akhremitchev, B. B., Effects of Multiple-Bond Ruptures on Kinetic Parameters Extracted from Force Spectroscopy Measurements: Revisiting Biotin-Streptavidin Interactions. Biophysical Journal 2008, 95 (8), 3964-3976.
20. Taninaka, A.; Takeuchi, O.; Shigekawa, H., Reconsideration of Dynamic Force Spectroscopy Analysis of Streptavidin-Biotin Interactions. International Journal of Molecular Sciences 2010, 11 (5), 2134-2151.
21. Rico, F.; et al., Heterogeneous and rate-dependent streptavidin-biotin unbinding revealed by high-speed force spectroscopy and atomistic simulations. Proceedings of the National Academy of Sciences 2019, 116 (14), 6594-6601.
22. Cenev, Z.; Zhang, H.; Sariola, V.; Rahikkala, A.; Liu, D.; Santos, H. A.; Zhou, Q., Manipulating Superparamagnetic Microparticles with an Electromagnetic Needle. Advanced Materials Technologies 2018, 3 (1), 1700177.
23. Zacchia, N. A.; Valentine, M. T., Design and optimization of arrays of neodymium iron boron-based magnets for magnetic tweezers applications. Review of Scientific Instruments 2015, 86 (5), 053704.
24. Syed, F.; Grunenwald, H.; Caruccio, N., Next-generation sequencing library preparation: simultaneous fragmentation and tagging using in vitro transposition. Nat.

Methods 2009, 6 (11), i-ii.
25. Zhang, H.; et al., NAD tagSeq reveals that NAD(+)-capped RNAs are mostly produced from a large number of protein-coding genes in Arabidopsis. Proceedings of the National Academy of Sciences of the United States of America 2019, 116 (24), 12072-12077.
26. Slesarev, A.; et al., CRISPR/CAS9 targeted CAPTURE of mammalian genomic regions for characterization by NGS. Sci. Rep. 2019, 9 (1), 3587.
27. Ren, H.; et al., Novel target capture DNA library preparation method using CircLigase-mediated hook ligation. New Biotechnol. 2020, 59, 44-50.
28. Chan, W.-K.; et al., Oxidative amide synthesis and N-terminal α-amino group ligation of peptides in aqueous medium. Journal of the American Chemical Society 2006, 128 (46), 14796-14797.
29. Chan, A. et al., Modification of N-terminal α-amino groups of peptides and proteins using ketenes. Journal of the American Chemical Society 2012, 134 (5), 2589-2598.
30. Kung, K. K.-Y.; et al., N-terminal α-amino group modification of peptides by an oxime formation-exchange reaction sequence. Chemical Communications 2013, 49 (61), 6888-6890.
31. MacDonald, J. I.; Munch, H. K.; Moore, T.; Francis, M. B., One-step site-specific modification of native proteins with 2-pyridinecarboxyaldehydes. Nature chemical biology 2015, 11 (5), 326-331.
32. Chen, D.; et al., Selective N-terminal functionalization of native peptides and proteins. Chem Sci 2017, 8 (4), 2717-2722.
33. Biswas, S.; Song, W.; Borges, C.; Lindsay, S.; Zhang, P., Click addition of a DNA thread to the N-termini of peptides for their translocation through solid-state nanopores. ACS nano 2015, 9 (10), 9652-9664.
34. Xu, G.; Shin, S. B. Y.; Jaffrey, S. R., Chemoenzymatic labeling of protein C-termini for positive selection of C-terminal peptides. ACS chemical biology 2011, 6 (10), 1015-1020.
35. Hoyt, E. A.; et al., Contemporary approaches to site-selective protein modification. Nature Reviews Chemistry 2019, 3 (3), 147-171.
36. Bloom, S.; Liu, C.; Kolmel, D. K.; Qiao, J. X.; Zhang, Y.; Poss, M. A.; Ewing, W. R.; MacMillan, D. W., Decarboxylative alkylation for site-selective bioconjugation of native proteins via oxidation potentials. Nature chemistry 2018, 10 (2), 205-211.
37. Yu, Y.; et al., Chemoselective peptide modification via photocatalytic tryptophan β-position conjugation. Journal of the American Chemical Society 2018, 140 (22), 6797-6800.
38. Tan, D. J. Y.; et al., A modular approach to enzymatic ligation of peptides and proteins with oligonucleotides. Chemical Communications 2021, 57 (45), 5507-5510.
39. Mao, H.; Hart, S. A.; Schink, A.; Pollok, B. A., Sortase-mediated protein ligation: a new method for protein engineering. J Am Chem Soc 2004, 126 (9), 2670-1.
40. Hemu, X.; et al., Turning an Asparaginyl Endopeptidase into a Peptide Ligase. ACS Catalysis 2020, 10 (15), 8825-8834.
41. Nuijens, T.; T et al., Natural Occurring and Engineered Enzymes for Peptide Ligation and Cyclization. Frontiers in Chemistry 2019, 7 (829).
42. Maglia, G.; et al., Enhanced translocation of single DNA molecules through α-hemolysin nanopores by manipulation of internal charge. Proceedings of the National Academy of Sciences 2008, 105 (50), 19720-19725.
43. ELECTRONIC BIOSCIENCES, I., U.S. Ser. No. 10/047,129B2—Modified alpha hemolysin polypeptides and methods of use 2018.
44. Asandei, A.; et al., Electroosmotic trap against the electrophoretic force near a protein nanopore reveals peptide dynamics during capture and translocation. ACS applied materials & interfaces 2016, 8 (20), 13166-13179.
45. Huang, G.; Willems, K.; Soskine, M.; Wloka, C.; Maglia, G., Electro-osmotic capture and ionic discrimination of peptide and protein biomarkers with FraC nanopores. Nature communications 2017, 8 (1), 1-11.
46. Firnkes, M.; Pedone, D.; Knezevic, J.; Doblinger, M.; Rant, U., Electrically facilitated translocations of proteins through silicon nitride nanopores: conjoint and competitive action of diffusion, electrophoresis, and electroosmosis. Nano letters 2010, 10 (6), 2162-2167.
47. Meier, W.; Graff, A.; Diederich, A.; Winterhalter, M., Stabilization of planar lipid membranes: A stratified layer approach. Physical Chemistry Chemical Physics 2000, 2 (20), 4559-4562.
48. Burden, D. L.; et al., Mechanically Enhancing Planar Lipid Bilayers with a Minimal Actin Cortex. Langmuir 2018, 34 (37), 10847-10855.
49. Daly, S. M.; et al., Photopolymerization of Mixed Monolayers and Black Lipid Membranes Containing Gramicidin A and Diacetylenic Phospholipids. Langmuir 2006, 22 (3), 1215-1222.
50. Heitz, B. A.; et al., Polymerized Planar Suspended Lipid Bilayers for Single Ion Channel Recordings: Comparison of Several Dienoyl Lipids. Langmuir 2011, 27 (5), 1882-1890.
51. Heitz, B. A.; Xu, J.; Hall, H. K.; Aspinwall, C. A.; Saavedra, S. S., Enhanced Long-Term Stability for Single Ion Channel Recordings Using Suspended Poly(lipid) Bilayers. Journal of the American Chemical Society 2009, 131 (19), 6662-6663.
52. Bright, L. K.; Baker, C. A.; Bränström, R.; Saavedra, S. S.; Aspinwall, C. A., Methacrylate Polymer Scaffolding Enhances the Stability of Suspended Lipid Bilayers for Ion Channel Recordings and Biosensor Development. ACS Biomaterials Science & Engineering 2015, 1 (10), 955-963.
53. Won, Y.-Y.; Davis, H. T.; Bates, F. S., Giant Wormlike Rubber Micelles. Science 1999, 283 (5404), 960-963.
54. Timp, W.; Comer, J.; Aksimentiev, A., DNA Base-Calling from a Nanopore Using a Viterbi Algorithm. Biophysical Journal 2012, 102 (10), L37-L39.
55. Schreiber, J.; Karplus, K., Analysis of nanopore data using hidden Markov models. Bioinformatics 2015, 31 (12), 1897-1903.
56. Magi, A.; S et al., Nanopore sequencing data analysis: state of the art, applications and challenges. Briefings in Bioinformatics 2017, 19 (6), 1256-1272.
57. Boia, V.; et al., DeepNano: Deep recurrent neural networks for base calling in MinION nanopore reads. PLOS ONE 2017, 12 (6), e0178751.
58. Rang, F. J.; Kloosterman, W. P.; de Ridder, J., From squiggle to basepair: computational approaches for improving nanopore sequencing read accuracy. Genome Biology 2018, 19 (1), 90.
59. Smith, M.; et al., Evaluating the effectiveness of ensemble voting in improving the accuracy of consensus signals produced by various DTWA algorithms from step-current signals generated during nanopore sequencing. PLOS Computational Biology 2021, 17 (9), e1009350.
60. Silvestre-Ryan, J.; Holmes, I., Pair consensus decoding improves accuracy of neural network basecallers for nanopore sequencing. Genome Biology 2021, 22 (1), 38.

The entirety of each patent, patent application, publication and document referenced herein is incorporated by reference. Citation of patents, patent applications, publications and documents is not an admission that any of the foregoing is pertinent prior art, nor does it constitute any admission as to the contents or date of these publications or documents. Their citation is not an indication of a search for relevant disclosures. All statements regarding the date(s) or contents of the documents is based on available information and is not an admission as to their accuracy or correctness.

The technology has been described with reference to specific implementations. The terms and expressions that have been utilized herein to describe the technology are descriptive and not necessarily limiting. Certain modifications made to the disclosed implementations can be considered within the scope of the technology. Certain aspects of the disclosed implementations suitably may be practiced in the presence or absence of certain elements not specifically disclosed herein.

Each of the terms “comprising,” “consisting essentially of,” and “consisting of” may be replaced with either of the other two terms. The term “a” or “an” can refer to one of or a plurality of the elements it modifies (for example, “a reagent” can mean one or more reagents) unless it is contextually clear either one of the elements or more than one of the elements is described. The term “about” as used herein refers to a value within 10% of the underlying parameter (i.e., plus or minus 10%; for example, a weight of “about 100 grams” can include a weight between 90 grams and 110 grams). Use of the term “about” at the beginning of a listing of values modifies each of the values (for example, “about 1, 2 and 3” refers to “about 1, about 2 and about 3”). When a listing of values is described the listing includes all intermediate values and all fractional values thereof (for example, the listing of values “80%, 85% or 90%” includes the intermediate value 86% and the fractional value 86.4%). When a listing of values is followed by the term “or more,” the term “or more” applies to each of the values listed (for example, the listing of “80%, 90%, 95%, or more” or “80%, 90%, 95% or more” or “80%, 90%, or 95% or more” refers to “80% or more, 90% or more, or 95% or more”). When a listing of values is described, the listing includes all ranges between any two of the values listed (for example, the listing of “80%, 90% or 95%” includes ranges of “80% to 90%,” “80% to 95%” and “90% to 95%”).

Certain implementations of the technology are set forth in the claim that follows.

What is claimed is:

1. A nanopore polymer sequencing system, comprising:
a chip comprising a substrate, a layer disposed on the substrate, a sensor site disposed in the layer, an electrode disposed at the sensor site, an amplifier in connection with the electrode, a membrane disposed over the layer at the sensor site, and a nanopore disposed in the membrane;
a magnet disposed in a cis position relative to the nanopore; and
a circuit comprising the electrode and a reference electrode, an amplifier in connection with the electrode and the reference electrode, a data acquisition (DAQ) system in connection with the amplifier, a bias module in connection with the amplifier, and a control module in connection with the bias module and the DAQ system; wherein the circuit is configured to maintain a voltage bias level at the sensor site based on a state of the nanopore.

2. The system of claim 1, wherein:
the magnet is disposed on a moveable mount capable of assuming multiple positions, and
one of the positions is in magnetic field proximity to the nanopore; and
the magnet optionally is a magnetic tweezer;
the magnet optionally comprises a permanent magnet, a ferromagnetic material, a rare earth magnetic material;
the magnet optionally comprises magnet elements disposed in a side-by-side orientation;
the magnet optionally comprises one magnet or two or more magnets each separated by a distance;
the magnet optionally comprises an electromagnet or one or more electromagnet elements;
the system optionally comprises a Mu-metal strip disposed on a chip adjacent to a well; or
the system optionally comprises a magnetic tip or a focusing yoke positioned adjacent to the magnet.

3. The system of claim 1, wherein the circuit is configured to automatically control the voltage bias level at the sensor site in response to the state of the nanopore detected by the control module.

4. The system of claim 3, wherein the control module of the circuit comprises one or more of: a field-programmable gate array (FPGA), a microprocessor, memory, a microcontroller, a computer, an application specific integrated circuit (ASIC) and a fixed hardware circuit.

5. The system of claim 3, wherein:
the state of the nanopore is chosen from one or more of: no polymer in association with the nanopore or a polymer translocation event; and
the translocation event is a polymer capture event, polymer exit event or polymer reentry event.

6. The system of claim 5, wherein:
the system comprises a chamber disposed in a trans position to the nanopore;
the chamber optionally comprises a hard stop agent capable of forming a distal hard stop structure on a magnetic particle-polymer conjugate when the polymer is disposed in the nanopore; and
the translocation event optionally is a distal hard stop and nanopore interaction event.

7. The system of claim 5, wherein:
the circuit is configured to iteratively apply a voltage bias at the sensor site, wherein each iteration comprises a voltage bias modification; and
the circuit optionally is configured to iteratively apply a voltage bias modification at the sensor site in response to a polymer translocation event detected by the control module.

8. The system of claim 3, wherein:
the control module is configured to detect one or more of a polymer capture event, polymer exit event, polymer reentry event and distal hard stop and nanopore interaction event; and
one or more of the polymer capture event, polymer exit event, polymer reentry event and distal hard stop and nanopore interaction event optionally occur at predetermined set points stored in the control module.

9. The system of claim 8, wherein the circuit comprises a multiplexer circuit and the control module is configured to transmit to the multiplexer circuit a change in the voltage bias level to (i) a holding voltage bias level after detecting the polymer capture event, or (ii) an exit voltage bias level after detecting the polymer capture event, or (iii) a reentry voltage bias level after detecting the polymer exit event, or (iv) an exit voltage bias level after detecting the polymer reentry event, or (v) a reentry voltage bias level after detecting the distal hard stop and nanopore interaction event, or (vi) a distal hard stop stripping voltage bias after detecting the distal hard stop and nanopore interaction event, or a combination of two, three or all of (i), (ii), (iii), (iv), (v) and (vi).

10. The system of claim 1, wherein layer is a passivated layer, or the membrane is a passivated membrane, or the layer is a passivated layer and the membrane is a passivated membrane, and:
the membrane optionally is a bilayer membrane, lipid membrane, black lipid membrane, block copolymer membrane, diblock copolymer membrane, dual block copolymer membrane or triblock copolymer membrane,
the membrane optionally comprises amphiphilic molecules;
the membrane optionally comprises amphiphilic molecules chosen from one or more of lipids, phospholipids, block copolymers, diblock copolymers, triblock copolymers, fatty acids, surfactants and polypeptides;
the membrane optionally is a planar lipid bilayer (PLB) containing lipids;
lipids in the PLB optionally contain a headgroup that reduces an attractive interaction between the PLB and the magnetic particle; and
lipids in the PLB optionally contain a headgroup associated with a passivating agent.

11. The system of claim 1, wherein the membrane is a crosslinked membrane.

12. The system of claim 1, comprising a conjugate, the conjugate comprising a polymer linked to a magnetic particle, wherein:
(i) the magnetic particle is passivated; or
(ii) the polymer comprises an affinity tag, or a reactive group, or an affinity tag and a reactive group, disposed at a distal portion or distal terminus of the polymer, wherein the affinity tag is capable of associating with a complementary affinity tag on a separate molecule and the reactive group is capable of associating with a complementary reactive group on a separate molecule; or
(iii) a combination of (i) and (ii).

13. The system of claim 12, wherein:
one or more of the magnetic particle of the conjugate, the layer and the membrane are passivated;
one or more of the magnetic particle, the layer and the membrane optionally are passivated with polyethylene glycol (PEG); and
the PEG optionally is of a molecular weight of about 0.1 kDa to about 100 kDa, or about 1 kDa to about 10 kDa, or about 5 kDa.

14. A method for translocating a polymer through a nanopore, comprising:
(a) contacting a polymer-magnetic particle conjugate with a system comprising a chip, wherein:
the polymer of the conjugate comprises a proximal end and a distal end;
the proximal end of the polymer is attached to the magnetic particle;
the chip comprises a nanopore disposed in a membrane;
the nanopore comprises an orifice smaller than the magnetic particle;
the system comprises a magnet disposed in a cis orientation relative to the nanopore;
the system comprises a chamber disposed in a trans orientation relative to the nanopore;
the magnetic particle optionally is, or the membrane optionally is, or the magnetic particle and the membrane optionally are, passivated; and
the membrane optionally is a crosslinked membrane;
(b) exerting an electrophoretic and/or electroosmotic force on the conjugate, in a trans direction, sufficient to dispose a portion of the polymer in the nanopore;
(c) after (b), exerting (i) a magnetic force on the conjugate in a cis direction and (ii) an electrophoretic and/or electroosmotic force on the conjugate in a trans direction, thereby exerting a net force between the electrophoretic and/or electroosmotic force and the magnetic force on the conjugate, wherein the net force translocates the polymer of the conjugate in the cis direction.

15. The method of claim 14, wherein the chamber comprises a hard stop agent, and part (b) is performed under conditions in which the hard stop agent associates with a distal portion or distal terminus of the polymer in the chamber, thereby forming a distal hard stop structure, wherein a hydrodynamic diameter of the hard stop structure is larger than a constriction region diameter of the nanopore.

16. The method of claim 14, comprising (d), after (c), iteratively modulating the net force on the conjugate and thereby iteratively translocating the polymer of the conjugate in opposing directions through the nanopore, wherein in an iteration the polymer of the conjugate translocates through the nanopore in a direction different than the direction in a preceding iteration.

17. The method of claim 16, wherein the polymer translocates through the nanopore (i) without modifying the magnetic field applied by the magnet, or (ii) without altering the position of the magnet, or (iii) without the conjugate contacting the magnet, or a combination of two or three of (i), (ii) and (iii).

18. The method of claim 16, wherein:
in (c) and (d) the polymer is elongated;
the polymer in (a) optionally contains secondary structure and/or tertiary structure, and the net force on the conjugate in (c) exceeds a force required to disrupt secondary structure and tertiary structure in the polymer; and
a tensile force is exerted on the polymer in (c) and (d), and the tensile force optionally is about 0.1 to about 200 picoNewtons (pN).

19. A nucleic acid, comprising a central nucleic acid flanked by a terminal proximal region and a terminal distal region, wherein:
the proximal region comprises a magnetic particle; and
the distal region comprises a terminal single stranded polynucleotide;
the terminal single stranded polynucleotide of the distal region comprises a polynucleotide having the structure (X)ₙ, wherein X is a nucleotide and n is about 10 to about 80 consecutive nucleotides;
the central nucleic acid optionally is partially single stranded nucleic acid and/or partially double stranded;
in the proximal region, the magnetic particle optionally is linked to the first strand of the central nucleic acid, and the second strand of the central nucleic acid optionally is linked to a terminal blocking moiety;
the proximal terminal end of the single stranded polynucleotide, the magnetic particle and/or the proximal terminal blocking moiety optionally are independently linked to an adapter nucleic acid in the proximal region; and
the distal terminal end of the single stranded polynucleotide and/or the distal terminal blocking moiety optionally are independently linked to an adapter nucleic acid in the distal region.

20. A method for preparing a modified nucleic acid that includes a central nucleic acid region, a proximal region on one side of the central nucleic acid region and a distal region on the other side of the central nucleic acid region, the method comprising:
providing an input nucleic acid;
linking a magnetic particle to a proximal end of the input nucleic acid; and
linking a terminal single stranded polynucleotide to a distal end of the input nucleic acid, thereby generating a nucleic acid comprising (i) a central nucleic acid region comprising the input nucleic acid, (ii) a proximal region comprising the magnetic particle, and (iii) a distal region comprising the terminal single stranded polynucleotide.
